(19) United States
(12) Patent Application Publication
(10) Pub. No.: US 2009/0215086 A1
(43) Pub. Date: Aug. 27, 2009

(54) Biomarkers for Babesia

(76) Inventors: Momar Ndao, La Prairie (CA); Brian Ward, Montreal (CA); Peter Krause, Hartford, CT (US); Mark W. Duncan, Denver, CO (US); Mike Edwards, Denver, CO (US); Terence William Spithill, Notre Dame D'lle Perrot (CA)

Correspondence Address: FENWICK & WEST LLP, SILICON VALLEY CENTER, 801 CALIFORNIA STREET, MOUNTAIN VIEW, CA, 94041, US

(21) Appl. No.: 12/097,037

(22) PCT Filed: Dec. 12, 2006
(86) PCT No.: PCT/US2006/061954

§ 371 Date: Mar. 23, 2009

Related U.S. Application Data
(60) Provisional application No. 60/749,449, filed on Dec. 12, 2005.

(51) Int. Cl. G01N 33/569

(52) U.S. Cl. 435/7.22

(57) ABSTRACT

The present invention provides protein-based biomarkers and biomarker combinations that are useful in qualifying babesia status in a patient. In particular, the biomarkers of this invention are useful to classify a subject sample as infected with babesia or not infected with babesia. The biomarkers can be detected by SELDI mass spectrometry.

### CROSS-REFERENCE TO RELATED APPLICATIONS

This application claims priority to U.S. Provisional Application Ser. No. 60/749,449 filed Dec. 12, 2005, incorporated herein by reference in its entirety.

### FIELD

The invention relates generally to clinical diagnostics.

### BACKGROUND

Babesiosis, also referred to as babesia, is a disease caused by ubiquitous protozoan parasites of the Babesia family. These parasites and closely-related species have a worldwide distribution and infect a wide range of mammals (Dao Compr Ther. 1996;22(11):713-8; Krause Med Clin North Am. 2002;86(2):361-73). The vast majority of human cases in North America are caused by the rodent parasite B Babesia microti. In other parts of the world, the cattle parasite B. divergens is implicated more often (Zintl et al. Clin Microbiol Rev. 2003;16(4):622-36). These parasites typically cause disease in relatively discreet areas related to the presence of the appropriate hard-bodied tick vectors (ixodid species). In North America, these ticks and humans ‘mingle’ primarily in the recreational and residential areas of the Northeast (eg: Maine to North Carolina), around the Great Lakes (including discreet regions of southern Ontario) and in more limited foci on the West Coast (Washington & California). Risk factors for infection include residence, recreation or work in one of these regions that results in exposure to tick habitat (Krause et al. J Clin Microbiol. 1991;29(l):1-4). In the USA, it has been estimated that hundreds of cases occur annually (Herwalt Strickland GT (ed) Hunter's Tropical Medicine and Emerging Infectious Diseases—English Edition. Saunders: Philadelphia 2000. Pp68890). Serosurveys conducted in Babesia-endemic regions suggest that infection rates may be much (Hunfeld et al. J Clin Microbiol. 2002;40(7):2431-6; Krause et al. Am J Trop Med Hyg. 2003;68(4):431-6). Babesia species are all readily transmitted by blood transfusion (Smith et al. Clin Lab Sci. 2003;16(4):239-45, 251, Dodd Int J Hematol. 2004;80(4):301-5) and transfusion-related cases have been reported in several jurisdictions around the world including the USA (Dodd Id) and Canada (Kain et al. CMAJ. 2001;164(12): 1721-3.). Similar foci of human infection with closely-related Babesia species can be found on several other continents including Europe and Asia (Gray Pol J Microbiol. 2004;53 Suppl:55-60, Ahmed et al. Parasitol Res. 2002;88(13 Suppl 1):S51-5) and it is very likely that this family of protozoa is ubiquitous but goes unrecognized in many regions of the world. Babesia species are major pathogens of cattle throughout the world (Herwalt In Strickland GT (ed) Hunter's Tropical Medicine and Emerging Infectious Diseases—Eighth Edition. Saunders: Philadelphia 2000. Pp68890.).

Babesiosis is normally a monophasic, malaria-like illness with an incubation period of one to several weeks and duration of 5-15 days (longer following blood transfusion). Like malaria, Babesia targets the human erythrocyte for replication. Unlike malaria, there is no acute or chronic liver stage of babesiosis and the parasite life-cycle in humans is restricted to the red blood cell (Herwalt Id, Krause et al. Med Clin North Am. 2002;86(2):361-73). Many subjects experience babesiosis infection with only mild and transient symptoms and the disease goes unrecognized. Typical symptoms are non-specific and include fever, chills and malaise. In the absence of a defined-tick exposure, the index of suspicion must be quite high to pursue the diagnosis in most subjects. In the large majority of subjects with defined babesiosis, recovery from infection is uncomplicated and complete. A small number of Babesia-infected subjects can suffer more severe and even fatal illness. The commonest risk factor for severe disease is anatomic or functional absence of the spleen (eg: post-splenctomy, sickle cell anemia). This organ is largely responsible for removing parasitized RBCs and, in its absence, parasitemia can reach very high levels resulting in massive hemolysis, end-organ failure and shock. Rarely, persistent parastemia can occur (Krause et al. Engl J Med. 1998;339(3):160-5). Furthermore, a small number of subjects with defined tick exposure and either serologic or microscopic confirmation of infection fail to resolve some or all of their symptoms after disappearance of the parasite from their RBC. Although these observations have caused some investigators to speculate about a chronic form of babesiosis (Sherr Med Hypotheses.2004;63(4):609-15), the mechanism(s) by which Babesia could persist are, as yet, poorly understood.

A definitive diagnosis of babesiosis is best made either by direct microscopic identification of parasitized RBCs or by seroconversion with acute and convalescent sera. Microscopic examination can be highly specific in skilled hands but this test is subject to both low sensitivity and low specificity under routine laboratory conditions (Krause 2002, Med Hypotheses. 2004;63(4):609-15). In North America, serologic testing is largely based on indirect immunofluorescence (IFA) performed by a limited number of laboratories in the USA (eg: CDC, some State laboratories in endemic areas). Furthermore, all IFA tests are notoriously operator-dependent (ie: subjective) and non-specific. As a result, single serologic measures for babesiosis often give confusing results.

Accordingly, presently, no optimal test is available for the diagnosis of babesia. Furthermore, there is currently no screening test for babesiosis that is appropriate for use in the blood system. Several EIA assays have been reported in the literature (Krause et al. J Infect Dis. 1994; 169(4):923-6, Houghton et al. Transfusion. 2002;42(11):1488-96, Loa et al. Curr Microbiol. 2004;49(6):385-9) as well as immunoblots (Ryan et al. Clin Diagn Lab Immunol. 2001 November;8(6):1177-80) but these assays have not yet been commercialized and likely suffer the same limitations of sensitivity and specificity as the IFA-based assays. A need exits for new methods of detecting babesiosis in a subject. This invention is directed to this and other ends.

### BRIEF SUMMARY

The present invention provides, inter alia, biomarkers that are differentially present in subjects with babesia. In addition, the present invention provides methods of using the biomarkers to qualify babesia in a subject or in a biological sample taken from a subject, including a sample of scrum, blood or other donated tissue. As such, the invention provides biomarkers that represent fill length proteins or fragments of proteins expressed in infected individuals by a parasite of the Babesia family, the pathogen responsible for babesia.

The biomarkers can be used, inter alia, to qualify babesia status, determine the course of babesia, monitor the response to treatment by a drug used to treat babesia, and /or determine a treatment regimen for babesia. The babesia can be caused by protozoan parasites of the Babesia microti family, the Babesia divergens family, or other species in the Babesia family.

In one aspect, the present invention provides a method for qualifying babesia status in a subject, the method comprising: (a) measuring at least one biomarker in a biological sample from the subject, wherein the at least one biomarker is selected from the group consisting of the biomarkers of Tables 1-3; and (b) correlating the measurement with babesia status. In one embodiment, the biological sample is a serum sample.

The at least one biomarker can be selected from the group consisting of biomarkers of molecular masses of about 2.8, 2.9, 3, 3.1, 3.2, 3.6, 3.8, 4, 4.1, 4.2, 4.3, 4.8, 4.9, 6.4, 7, 7.1, 7.2, 7.3, 7.5, 7.7, 7.9, 8.7, 8.8, 8.9, 10, 10.1, 10.2, 10.3, 10.4, 10.9, 11, 11.2, 11.3, 11.6, 11.8, 11.9, 12.6, 12.7, 12.8, 12.9, 13, 13.1, 13.2, 13.6, 13.8, 14.1, 14.4, 14.7, 15.1, 15.6, 15.9, 16.5, 16.7, 17.3, 17.8, 21.9, 22.2, 22.3, 23.5, 23.6, 25.5, 25.8, 28.1, 28.2, 33.1, 33.2, 33.3, 34.1, 36.1, 39.8, 43.4, 44.2, 44.3, 44.8, 45.1, 46.1, 47.7, 51, 53, 53.6, 60.6, 62.4, 66.9, 79, 18.1, 19.2, 22.3, 26.5, 39.6, 39.9, 40.1, 41.3, 43.2, 43.6, 44.2, 44.4, 44.6, 45.2, 44.7, 50, 50.5, 51.2, 51.5, 51.9, 52.5, 52.7, 58.9, 59.1, 59.6, 59.8, 60.5, 61.6, 61.9, 62.3, 62.8, 64, 66.3, 66.6, 78.5, 79, 79.2, 79.5, 79.6, 99.3, 99.6, 110.2, 131.8, 133.5, 134.6, 146.6, 167.8, 168, and 196.4 kDa and any combination thereof.

The at least one biomarker can be selected from the group consisting of biomarkers of molecular masses of about2.8, 2.9, 3, 3.2, 7.1, 7.2, 7.3, 7.5, 7.7, 7.9, 8.9, 14.1, 15.6, 39.8, 44.2, 53.6, 60.6, 62.4, and 79 kDa and any combination thereof. The at least one biomarker can be selected from the group consisting of biomarkers of molecular masses of about 11.8, 12.6, 12.7, 12.8, 12.9, 13, 13.1, 13.2, 18.1, 19.2, 26.5, 39.9, 43.6, 51.5, 59.8, 62.8, 79 and 146.6 kDa and any combination thereof. The at least one biomarker can be selected from the group consisting of biomarkers of molecular masses of about 2.9, 10.2, 10.3, 13.6, 14.7, 15.9, 43.2, 44.2, 44.4 and 79.6 kDa and any combination thereof. The at least one biomarker can be selected from the group consisting of biomarkers of molecular masses of about 16.5, 16.7, 39.6, 40.1, 41.3, 58.9, 59.6, 60.5, 61.9, 62.8, 64, 66.6, and 79.2 kDa and any combination thereof. The at least one biomarker can be selected from the group consisting of biomarkers of molecular masses of about 3.6, 4.8, 14.1, 28.1, and 28.2 kDa. The at least one biomarker can be selected from the group consisting of biomarkers of molecular masses of about 10.4, 23.5, 25.8, and 44.4 kDa and any combination thereof. The at least one biomarker can be selected from the group consisting of biomarkers of molecular masses of about 2.9, 3.8, 4.2, 4.9, 6.4, 7, 14.1, 25.5, 44.7, 45.2, 59.1, 61.6, 62.3, 79.5, and 99.6 kDa and any combination thereof. The at least one biomarker can be selected from the group consisting of biomarkers of molecular masses of about 13, 44.6, 133.5, and 168 kDa and any combination thereof. The at least one biomarker can be selected from the group consisting of biomarkers of molecular masses of about 3.1, 4, 4.1, 8.8, 34.1, 36.1, 44.8, 46.1, 47.7, and 66.9 kDa and any combination thereof. The at least one biomarker can be selected from the group consisting of biomarkers of molecular masses of about 10, 10.1, 10.9, 11, 11.2, 11.3, 11.6, 11.9, 43.4, 44.2, 50, 51.9, 52.5, and 134.6 kDa and any combination thereof. The at least one biomarker can be selected from the group consisting of biomarkers of molecular masses of about 4, 4.1, 6.4, 8.7, 14.4, 15.1, 17.3, 33.2, 45.1, and 53 kDa and any combination thereof. The at least one biomarker can be selected from the group consisting of biomarkers of molecular masses of about 10, 10.1, 12.6, 21.9, 22.3, 23.6, 33.1, 50.5, 51.2, 167.8 kDa and any combination thereof. The at least one biomarker can be selected from the group consisting of biomarkers of molecular masses of about 4.1 and 4.3 kDa and any combination thereof. The at least one biomarker can be selected from the group consisting of biomarkers of molecular masses of about 11.6, 17.8, 22.3, and 52.7 kDa and any combination thereof. The at least one biomarker can be selected from the group consisting of biomarkers of molecular masses of about 13.8, 22.2, 33.3, 44.3 kDa and any combination thereof. The at least one biomarker can be selected from the group consisting of biomarkers of molecular masses of about 51, 66.3, 78.5, 99.3, 110.2, 131.8, 196.4 kDa and any combination thereof. It will be understood that any combination of the biomarkers described herein can be measured using the methods described herein.

In some embodiments, the at least one biomarker is selected from the group consisting of biomarkers of molecular masses of about 22, 28, 33, 44, and 146 kDa and any combination thereof. In some embodiments, the at least one biomarker is selected from the group consisting of biomarkers of molecular masses of about 7.6, 8.9, 28.1 and 44.4 kDa and any combination thereof. In some embodiments, the at least one biomarker is selected from the group consisting of biomarkers of molecular masses of about 7.2 and 7.3 kDa and any combination thereof. In some embodiments, each of the biomarkers having a molecular mass of about 22, 28, 33, 44, and 146 kDa is measured.

In some embodiments, the at least one biomarker is selected from the group consisting of biomarkers of molecular masses of about 3, 4, 7, 15, 22, 36, 48, 51, 62, and 135 kDa and any combination thereof. In some embodiments, the at least one biomarker is selected from the group consisting of biomarkers of molecular masses of about 2.9, 3.6, 7, 14.7, 15.1, 22.3, 36.1, 47.7, 51.2, 51.5, 61.9, and 134.6 kDa and any combination thereof. In some embodiments, the at least one biomarker is selected from the group consisting of biomarkers of molecular masses of about 2.86, 3.57, 6.96, 14.72, 15.16, 22.29, 36.08, 47.71, 51.18, 51.54, 61.95, and 134.61 kDa and any combination thereof.

In some embodiments, the at least one biomarker is a protein or fragment thereof as provided in Table 3 and Table P. In certain embodiments, the at least one biomarker is represented by SEQ ID NOS:1-22.

In one embodiment, the at least one biomarker is measured by capturing the biomarker on an adsorbent of a SELDI probe and detecting the captured biomarkers by laser desorption-ionization mass spectrometry. In certain embodiments, the adsorbent is a cation exchange adsorbent, whereas in other embodiments, the adsorbent is a metal chelation adsorbent. In another embodiment, the at least one biomarker is measured by immunoassay.

In another embodiment, the correlating is performed by a software classification algorithm. In a further embodiment, babesia status is selected from chronically infected versus uninfected. In yet another embodiments, babesia status is selected from chronically infected status versus acutely infected disease status, chronically infected asymptomatic status versus chronically affected with symptoms, or acutely infected status versus healthy uninfected status. In still another embodiment, babesia status is selected from babesia versus healthy.

In yet another embodiment, the method further comprises managing subject treatment based on the status. If the measurement correlates with babesia, then managing subject treatment comprises administering to a patient drugs selected from a group consisting of, but not necessarily limited to, drugs such as quinine, clindamycin and combinations thereof.

In a further embodiment, the method further comprises measuring the at least one biomarker after subject management.

In another aspect, the present invention provides a method comprising measuring at least one biomarker in a sample from a subject, wherein the at least one biomarker is selected from the group consisting of the biomarkers set forth in Tables 1-3. In one embodiment, the sample is a serum sample.

In still another aspect, the present invention provides a kit comprising: (a) a solid support comprising at least one capture reagent attached thereto, wherein the capture reagent binds at least one biomarker from a first group consisting of the biomarkers set forth in Table 1, Table 2 and Table 3; and (b) instructions for using the solid support to detect the at least one biomarker set forth in Table 1, Table 2 and Table 3.

In other embodiments, the kit additionally comprises (c) a container containing at least one of the biomarkers of Table 1, Table 2 and Table 3.

In yet a further aspect, the present invention provides a software product, the software product comprising: (a) code that accesses data attributed to a sample, the data comprising measurement of at least one biomarker in the sample, the biomarker selected from the group consisting of the biomarkers of Table 1, Table 2 and Table 3; and (b) code that executes a classification algorithm that classifies babesia status of the sample as a function of the measurement.

In one embodiment, the classification algorithm classifies babesia status of the sample as a function of the measurement of a biomarker selected from the biomarkers of Tables 1-3.

In other aspects, the present invention provides purified biomolecules selected from the biomarkers set forth in Table 1, Table 2 and Table 3 and, additionally, methods comprising detecting a biomarker set forth in Table 1, Table 2 and Table 3 by mass spectrometry or immunoassay.

In yet another embodiment, the method further comprises testing and qualifying stocks of blood based on the status of blood which has been tested according to the methods described herein. If the measurements taken from blood samples correlate with babesia, then the management of blood stocks comprises decontamination of the infected blood by treatment of the infected blood with purification agents available to one skilled in the art. Alternatively, the infected blood may be discarded or destroyed and only stocks of blood which have not tested positively for babesia are retained.

In one aspect, the present invention provides a method for qualifying babesia status in a subject in comparison to the status of a different parasitic, the method comprising: (a) measuring at least one biomarker in a biological sample from the subject, wherein the at least one biomarker specifically indicates the presence of babesia and does not indicate the presence of a different parasitic infection; and (b) correlating the measurement with babesia status in comparison to the status of a different parasitic infection. In one embodiment, the biological sample is a serum sample. In a preferred embodiment of this method, the at least one biomarker is selected from the group of biomarkers of Table 1-3. In still another preferred embodiment, the parasitic infection includes, but is not limited to, African trypanosomiasis (sleeping sickness), malaria and Chagas disease.

In another aspect, the present invention provides a method for monitoring the course of progression of babesia in a patient comprising: (a) measuring at least one biomarker in a first biological sample from the patient, wherein the at least one biomarker specifically indicates the presence of babesia; (b) measuring the at least one biomarker in a second biological sample from the subject, wherein the second biological sample was obtained from the subject after the first biological sample; and (c) correlating the measurements with the progression or regression of babesia in the subject. In one embodiment, the at least one biomarker is selected from the group consisting of the biomarkers of Tables 1-3.

Other features, objects and advantages of the invention and its preferred embodiments will become apparent from the detailed description, examples and claims that follow.

### BRIEF DESCRIPTION OF THE DRAWINGS

FIG. 1 shows a graphical representation of the differential signal intensity of the 7.2 and 7.3 kDa biomarkers in subjects having babesia versus healthy subjects, subjects having African trypanosomiasis (sleeping sickness), subjects having Chagas Disease, and subjects having Malaria. The biomarkers are characterized by their mass-to-charge ratio as determined by mass spectrometry.

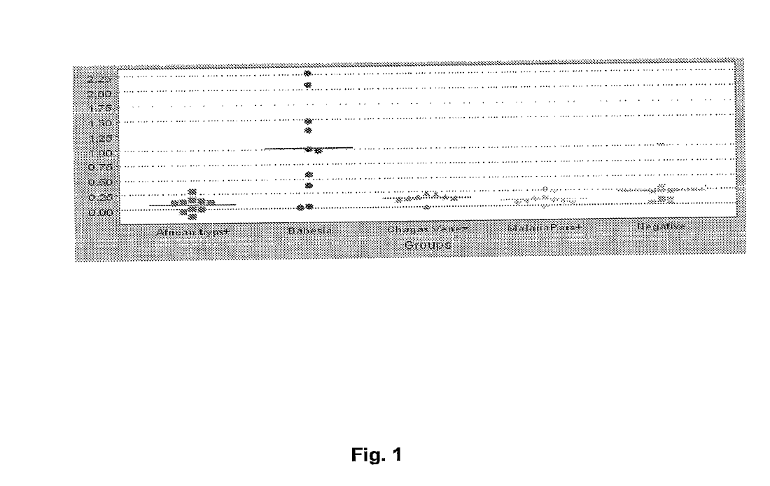

FIG. 2 provides a scanned gel image of total control versus total babesia infected sera.

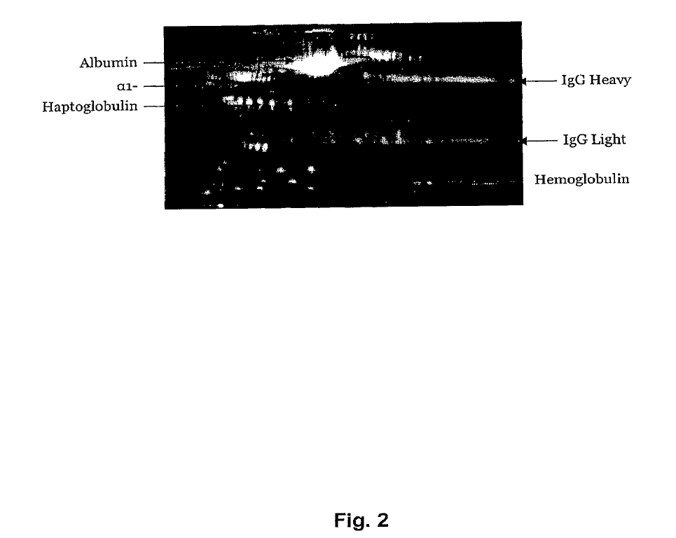

FIG. 3 shows a representation of the differential signal intensity of the 51.5 kDA biomarker in subjects having babesia versus non-babesia (control), subjects having babesia versus healthy subjects, and subjects having babesia versus subjects having flu-like symptoms. The biomarker is characterized by its mass-to-charge ratio as determined by mass spectrometry and is in daltons.

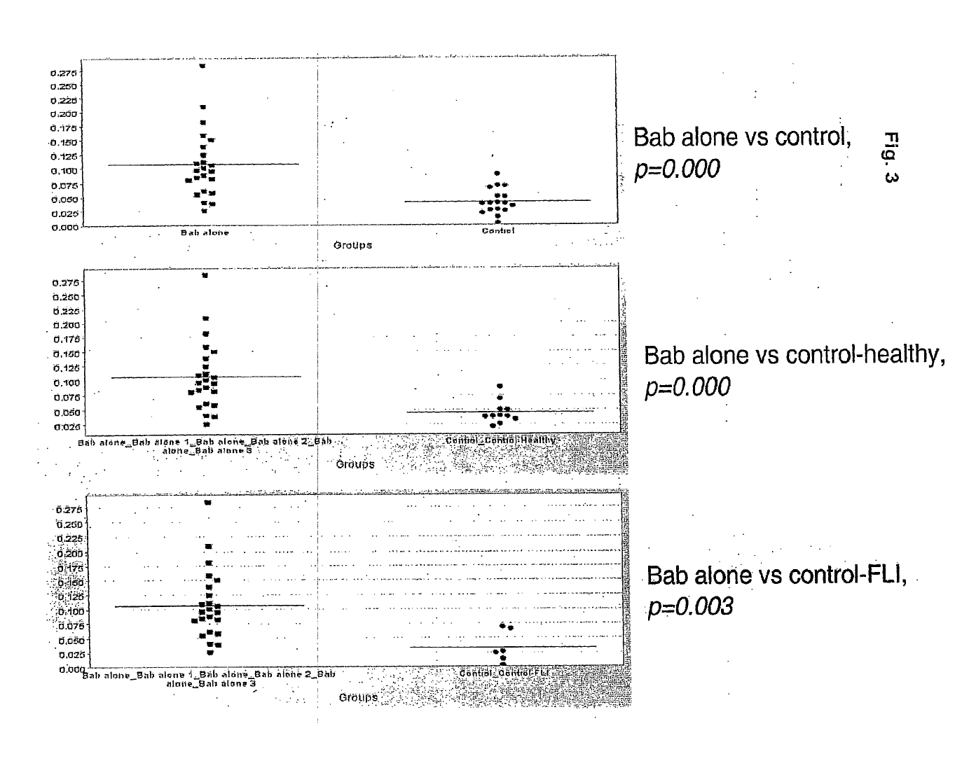

FIG. 4 shows a representation of the differential signal intensity of the 14.7 kDA biomarker in subjects having babesia versus non-babesia (control), subjects having babesia versus healthy subjects, and subjects having babesia versus subjects having flu-like symptoms. The biomarker is characterized by its mass-to-charge ratio as determined by mass spectrometry and is in daltons.

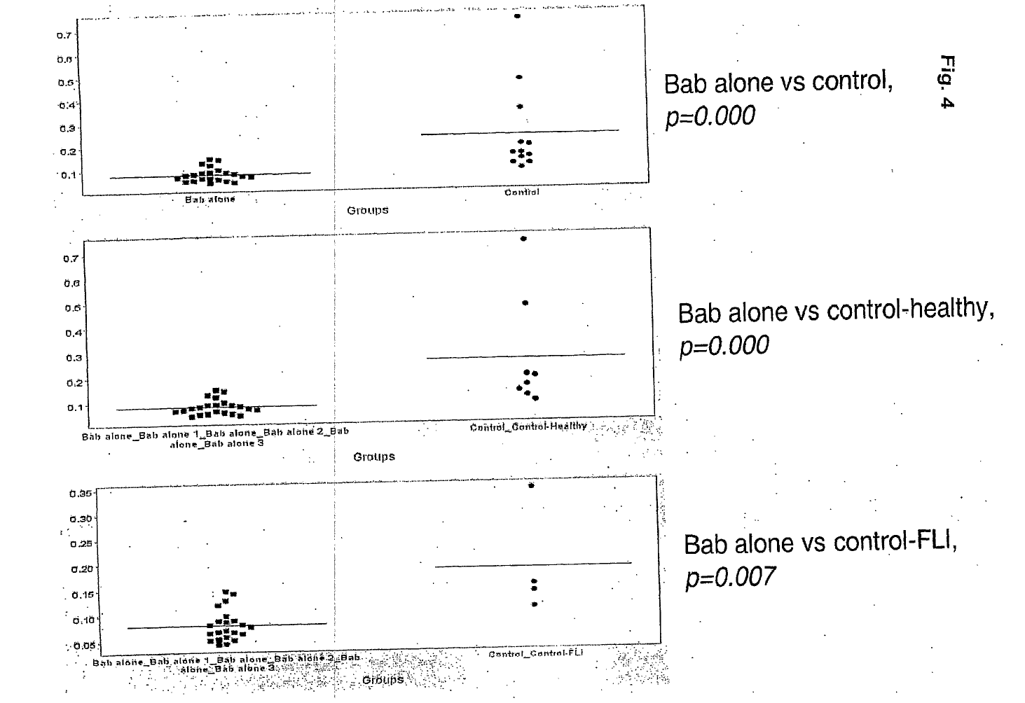

FIG. 5 shows a representation of the differential signal intensity of the 61.9 kDA biomarker in subjects having babesia versus non-babesia (control), subjects having babesia versus healthy subjects, and subjects having babesia versus subjects having flu-like symptoms. The biomarker is characterized by its mass-to-charge ratio as determined by mass spectrometry and is in daltons.

FIG. 6 shows a representation of the differential signal intensity of the 7 kDA biomarker in subjects having babesia versus non-babesia (control), subjects having babesia versus healthy subjects, and subjects having babesia versus subjects having flu-like symptoms. The biomarker is characterized by its mass-to-charge ratio as determined by mass spectrometry and is in daltons.

FIG. 7 shows a representation of the differential signal intensity of the 36.1 kDA biomarker in subjects having babesia versus non-babesia (control), subjects having babesia versus healthy subjects, and subjects having babesia versus subjects having flu-like symptoms. The biomarker is characterized by its mass-to-charge ratio as determined by mass spectrometry and is in daltons.

FIG. 8 shows a representation of the differential signal intensity of the 47.7 kDA biomarker in subjects having babesia versus non-babesia (control), subjects having babesia versus healthy subjects, and subjects having babesia versus subjects having flu-like symptoms. The biomarker is characterized by its mass-to-charge ratio as determined by mass spectrometry and is in daltons.

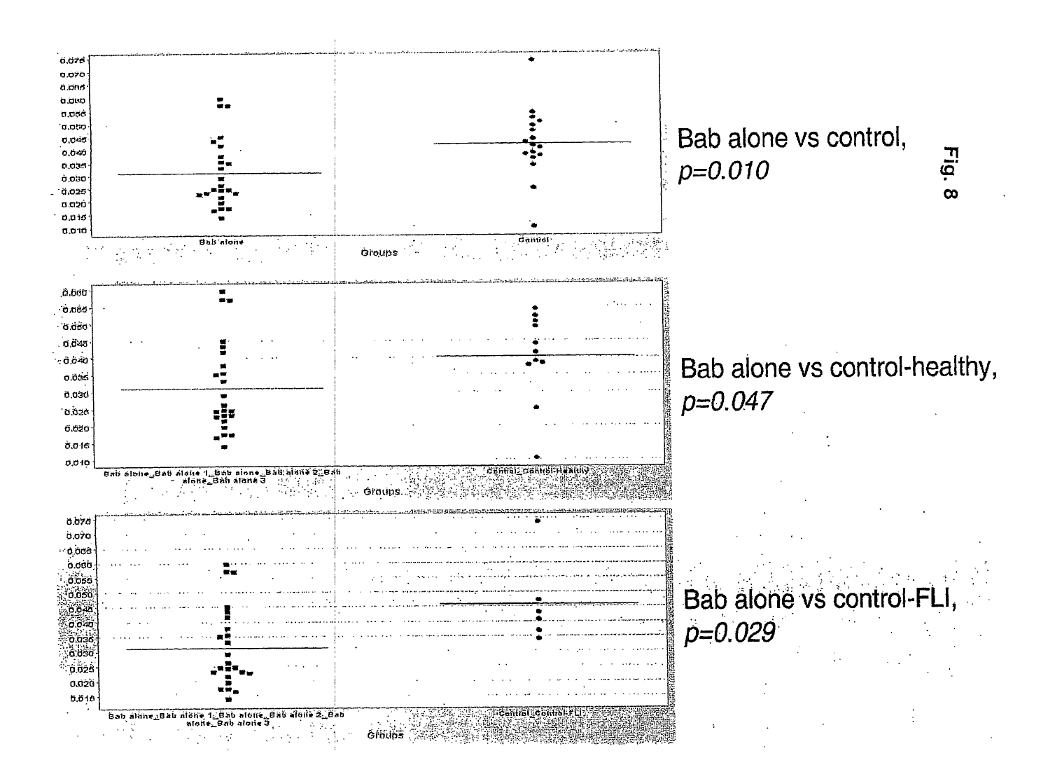

FIG. 9 shows a representation of the differential signal intensity of the 134.6 kDA biomarker in subjects having babesia versus non-babesia (control), subjects having babesia versus healthy subjects, and subjects having babesia versus subjects having flu-like symptoms. The biomarker is characterized by its mass-to-charge ratio as determined by mass spectrometry and is in daltons.

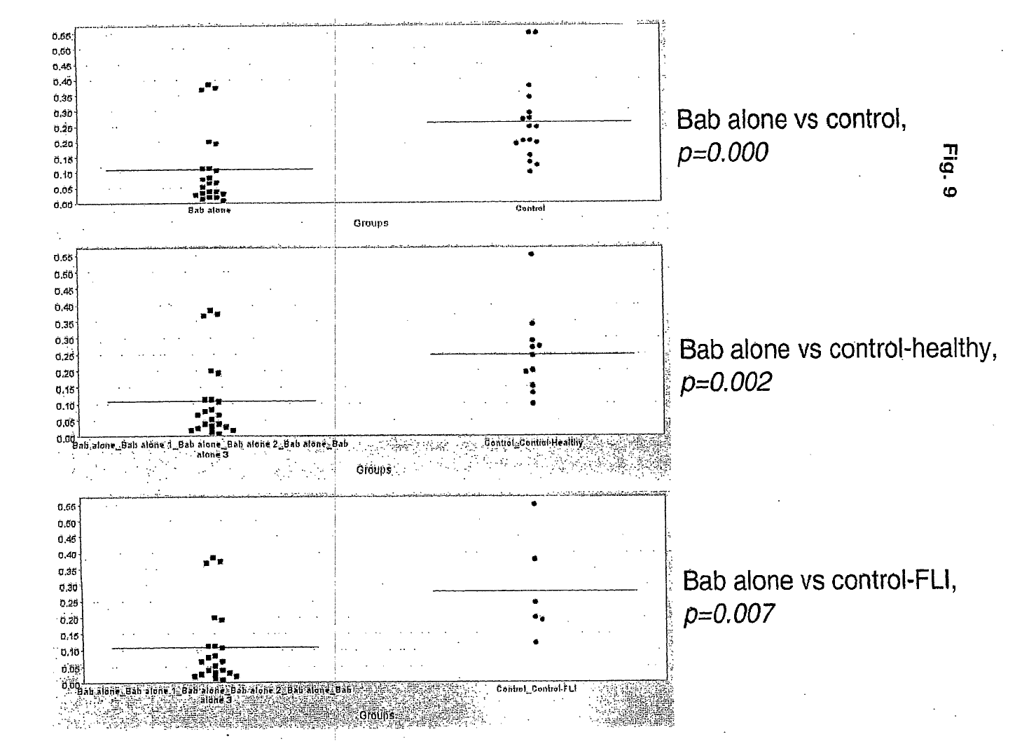

FIG. 10 shows a representation of the differential signal intensity of the 15.2 kDA biomarker in subjects having babesia versus non-babesia (control), subjects having babesia versus healthy subjects, and subjects having babesia versus subjects having flu-like symptoms. The biomarker is characterized by its mass-to-charge ratio as determined by mass spectrometry and is in daltons.

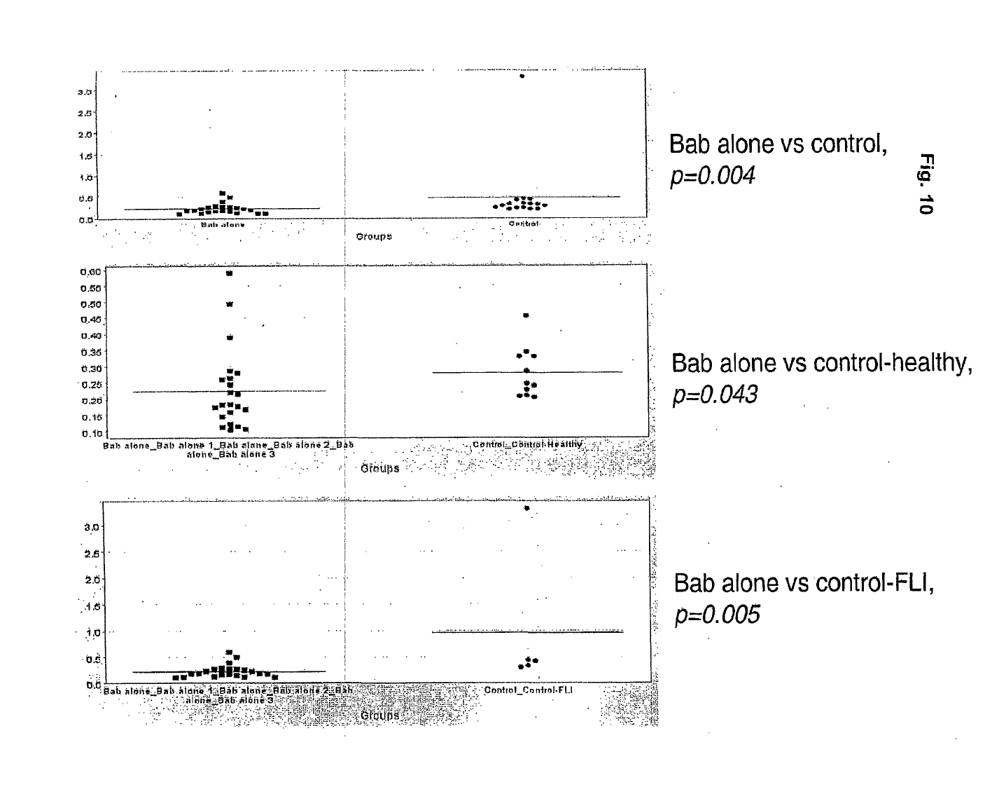

FIG. 11 shows a representation of the differential signal intensity of the 51.2 kDA Biomarker in subjects having babesia versus non-babesia (control), subjects having babesia versus healthy subjects, and subjects having babesia versus subjects having flu-like symptoms. The biomarker is characterized by its mass-to-charge ratio as determined by mass spectrometry and is in daltons.

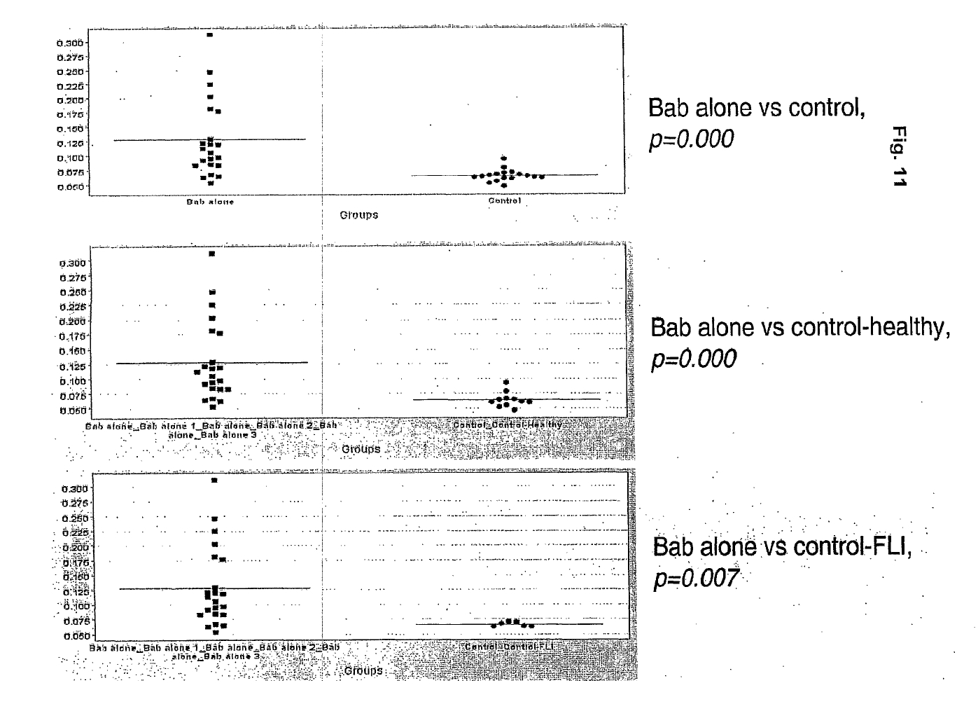

FIG. 12 shows a representation of the differential signal intensity of the 22.3 kDA biomarker in subjects having babesia versus non-babesia (control), subjects having babesia versus healthy subjects, and subjects having babesia versus subjects having flu-like symptoms. The biomarker is characterized by its mass-to-charge ratio as determined by mass spectrometry and is in daltons.

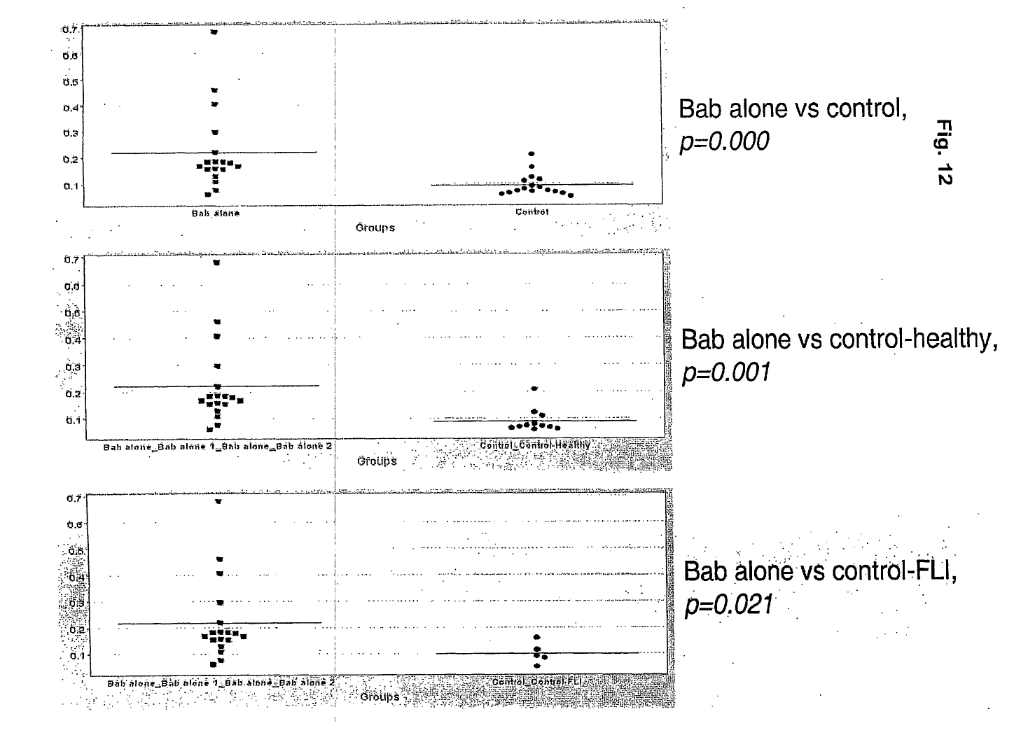

### DETAILED DESCRIPTION

### 1. Introduction

A biomarker is an organic biomolecule which is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics) and drug toxicity.

### 2. Biomarkers for Babesia

2.1. Biomarkers

This invention provides, inter alia, polypeptide-based biomarkers that are differentially present in subjects having babesia, in particular, and particularly that are differentially expressed in subjects infected with babesia versus non uninfected individuals (e.g., control, healthy, benign condition or other disease state). The biomarkers are characterized by mass-to-charge ratio as determined by mass spectrometry, by the shape of their spectral peak in time-of-flight mass spectrometry and by their binding characteristics to adsorbent surfaces. These characteristics provide one method to determine whether a particular detected biomolecule is a biomarker of this invention. These characteristics represent inherent characteristics of the biomolecules and not process limitations in the manner in which the biomolecules are discriminated. In one aspect, this invention provides these biomarkers in isolated form.

The biomarkers of Tables 1 and 2 were discovered using SELDI technology employing ProteinChip® arrays from Ciphergen Biosystems, Inc. (Fremont, Calif.) (“Ciphergen”). Serum samples were collected from subjects diagnosed with babesia and subjects diagnosed as healthy as well as subjects diagnosed with other kinetoplastidae infections (Non-babesia), such as African sleeping sickness, Chagas disease, and malaria or other conditions such as lyme disease or a flu-like condition. The samples were fractionated by anion exchange chromatography. Fractionated samples were applied to SELDI biochips and spectra of polypeptides in the samples were generated by time-of-flight mass spectrometry on a Ciphergen PBS IIc mass spectrometer. The spectra thus obtained were analyzed by Ciphergen Express™ Data Manager Software with Biomarker Wizard and Biomarker Pattern Software from Ciphergen Biosystems, Inc. The mass spectra for each group were subjected to scatter plot analysis. A Mann-Whitney test analysis was employed to compare babesia and control groups for each protein cluster in the scatter plot, and proteins were selected that differed significantly (p<0.05) between the two groups. This method is described in more detail in the Example Section.

The biomarkers thus discovered arc presented in Tables 1 and 2 (the protocol for the data obtained. is further described below and. in Section 9, in the Examples, under SELDI ANALYSIS). The heading of each column refers to chromatographic fraction in which the biomarker is found, the type of biochip to which the biomarker binds and the wash conditions.

TABLE 1

| F1CSL | F1CSH | F1ISL | F1ISH | F3CSL | F3CSH | F4ISL | F4ISH |
|---|---|---|---|---|---|---|---|
| 2.8 kDa | 11.8 kDa | 2.9 kDa | 16.5 kDa | 3.6 kDa | 10.4 kDa | 13.8 kDa | 51 kDa |
| 3 kDa | 12.6 kDa | 10.2 | 16.7 kDa | 4.8 kDa | 23.5 kDa | 22.2 kDa | 66.3 kDa |
| 3.2 kDa | 12.7 kDa | 10.3 | 39.6 kDa | 14.1 kDa | 25.8 kDa | 33.3 kDa | 78.5 kDa |
| 7.1 kDa | 12.8 kDa | 13.6 | 40.1 kDa | 28.1 kDa | 44.4 kDa | 44.3 kDa | 99.3 kDa |
| 7.2 kDa | 12.9 kDa | 14.7 kDa | 41.3 kDa | 28.2 kDa | | | 110.2 kDa |
| 7.3 kDa | 13 kDa | 15.9 kDa | 58.9 kDa | | | | 131.8 kDa |
| 7.5 kDa | 13.1 kDa | 43.2 kDa | 59.6 kDa | | | | 196.4 kDa |
| 7.7 kDa | 13.2 kDa | 44.2 kDa | 60.5 kDa | | | | |
| 8.9 kDa | 18.1 kDa | 44.4 kDa | 61.9 kDa | | | | |
| 14.1 kDa | 19.2 kDa | 79.6 kDa | 62.8 kDa | | | | |
| 15.6 kDa | 26.5 kDa | | 64 kDa | | | | |
| 39.8 kDa | 39.9 kDa | | 66.6 kDa | | | | |
| 44.2 kDa | 43.6 kDa | | 79.2 kDa | | | | |
| 53.6 kDa | 51.5 kDa | | | | | | |
| 60.6 kDa | 59.8 kDa | | | | | | |
| 62.4 kDa | 62.8 kDa | | | | | | |
| 79 kDa | 79 kDa | | | | | | |
| | 146.6 kDa | | | | | | |

* The biomarkers in bold were further identified as significant by Biomarker Patent Software

TABLE 2

| F5CSL | F5CSH | F5ISL | F5ISH | F6CSL | F6CSH | F6ISL | F6ISH |
|---|---|---|---|---|---|---|---|
| 2.9 kDa | 13 kDa | 3.1 kDa | 10 kDa | 4 kDa | 10 kDa | 4.1 kDa | 11.6 kDa |
| 3.8 kDa | 44.6 kDa | 4 kDa | 10.1 kDa | 4.1 kDa | 10.1 kDa | 4.3 kDa | 17.8 kDa |
| 4.2 kDa | 133.5 kDa | 4.1 kDa | 10.9 kDa | 6.4 kDa | 12.6 kDa | | 22.3 kDa |
| 4.9 kDa | 168 kDa | 8.8 kDa | 11 kDa | 8.7 kDa | 21.9 kDa | | 52.7 kDa |
| 6.4 kDa | | 34.1 kDa | 11.2 kDa | 14.4 kDa | 22.3 kDa | | |
| 7 kDa | | 36.1 kDa | 11.3 kDa | 15.1 kDa | 23.6 kDa | | |
| 14.1 kDa | | 44.8 kDa | 11.6 kDa | 17.3 kDa | 33.1 kDa | | |
| 25.5 kDa | | 46.1 kDa | 11.9 kDa | 33.2 kDa | 50.5 kDa | | |
| 44.7 kDa | | 47.7 kDa | 43.5 kDa | 45.1 kDa | 51.2 kDa | | |
| 45.2 kDa | | 66.9 kDa | 44.2 kDa | 53 kDa | 167.8 kDa | | |
| 59.1 kDa | | | 50 kDa | | | | |
| 61.6 kDa | | | 51.9 kDa | | | | |
| 62.3 kDa | | | 52.5 kDa | | | | |
| 79.5 kDa | | | 134.6 kDa | | | | |
| 99.6 kDa | | | | | | | |

* The biomarkers in bold were further identified as significant by Biomarker Patent Software

The biomarkers of Table 1 and Table 2 are characterized by their mass-to-charge ratio as determined by mass spectrometry. The mass-to-charge ratio of each biomarker in Table 1 and Table 2 are in kDa. The mass-to-charge ratios were determined from mass spectra generated on a Ciphergen Biosystems, Inc. PBS IIc mass spectrometer. This instrument has a mass accuracy of about +/−0.15 percent. Additionally, the instrument has a mass resolution of about 400 to 1000 m/dm, where m is mass and dm is the mass spectral peak width at 0.5 peak height. The mass-to-charge ratio of the biomarkers was determined using Biomarker Wizard™ software (Ciphergen Biosystems, Inc.). Biomarker Wizard assigns a mass-to-charge ratio to a biomarker by clustering the mass-to-charge ratios of the same peaks from all the spectra analyzed, as determined by the PBSIIc, taking the maximum and minimum mass-to-charge-ratio in the cluster, and dividing by two. Accordingly, the masses provided reflect these specifications.

The identity of certain of the biomarkers of Tables 1 and 2 of this invention has been determined and is indicated in Table P in the Examples section. For biomarkers whose identify has been determined, the presence of the biomarker can be determined by methods known in the art other than mass spectrometry.

The biomarkers of this invention can be further characterized by the shape of their spectral peak in time-of-flight mass spectrometry.

The biomarkers of this invention are further characterized by their binding properties on chromatographic surfaces.

The biomarkers of Table 3 were discovered using differential gel electrophoresis followed by protein identification by matrix-assisted laser desorption/ionization mass spectrometry (DIGE and MALDI-TOFMS). Serum samples were collected from subjects diagnosed with babesia and subjects diagnosed as normal (not having babesia). This method is described in more detail in the Example Section.

The biomarkers thus discovered are presented in Table 3.

TABLE 3

| | Avg. Ratio | Avg. Ratio |
|---|---|---|
| Protein | Total Pools | Sub-Pools |
| Alpha1-Antitrypsin (4 spots) | 3.95 ± 0.97 | 5.88 ± 5.35 |
| Chain A, Cleaved* (4 spots) | −2.16 ± 0.36 | −3.16 ± 2.26 |
| Haptoglobin | | |
| Beta Chain* (5 spots) | −1.46 ± 0.12 | −2.00 ± 1.04 |
| Alpha Chain (4 spots) | −1.81 ± 0.4 | −3.50 ± 2.77 |
| Hemoglobin Beta Chain (3 spots) | 1.90 ± 0.69 | 5.19 ± 3.68 |
| Apolipoprotein A-IV precursor | −2.12 ± 0.01 | −3.54 ± 2.79 |
| Unknown spot #940 | 2.61 ± 0.63 | absent |
| Proapolipoprotein* | −2.47 ± 0.78 | −3.35 ± 2.11 |
| Unknown spot #1098 | 1.70 ± 0.09 | 1.99 ± 0.33 |
| Immunoglobulins | | |
| gamma-1 heavy chain | 3.92 ± 1.29 | 3.99 |
| heavy constant alpha 1 | 1.45 ± 0.05 | 1.87 ± 0.58 |
| M heavy chain* | 1.50 ± 0.29 | 1.94 |
| CD5 Antigen-Like | 1.46 ± 0.14 | 1.85 ± 0.14 |
| Alpha2-HS Glycoprotein* | −2.26 ± 0.06 | −1.62 ± 0.53 |
| Complement Component 4A | 1.88 ± 0.69 | 3.52 |
| Unknown spot #1309 | −1.71 ± 0.13 | −2.00 |
| Vitamin D-Binding Protein | −1.84 ± 0.18 | −2.29 ± 0.77 |
| Human Serum Albumin (2 spots) | −1.92 ± 0.21 | −2.01 ± 0.61 |
| Complement Factor B* | −1.28 ± 0.14 | −1.52 ± 0.15 |
| Unknown spot #1560 | −1.92 ± 0.26 | −1.97 ± 0.1 |
| Annexin V | −1.47 ± 0.24 | absent |

The identity of certain of the biomarkers of Table 3 of this invention has been determined and is indicated in Table 3. For biomarkers whose identify has been determined, the presence of the biomarker can be determined by methods known in the art other than mass spectrometry.

Because the biomarkers of Tables 1 and 2 are characterized by mass-to-charge ratio and binding properties, they can be detected by mass spectrometry without knowing their specific identity. The identity of certain of the biomarkers of Tables 1-3 is known. If desired, biomarkers whose identity is not determined can be identified by, for example, determining the amino acid sequence of the polypeptides. For example, a biomarker can be peptide-mapped with a number of enzymes, such as trypsin or V8 protease, and the molecular weights of the digestion fragments can be used to search databases for sequences that match the molecular weights of the digestion fragments generated by the various enzymes. Alternatively, protein biomarkers can be sequenced using tandem MS technology. In this method, the protein is isolated by, for example, gel electrophoresis. A band containing the biomarker is cut out and the protein is subject to protease digestion. Individual protein fragments are separated by a first mass spectrometer. The fragment is then subjected to collision-induced cooling, which fragments the peptide and produces a polypeptide ladder. A polypeptide ladder is then analyzed by the second mass spectrometer of the tandem MS. The difference in masses of the members of the polypeptide ladder identifies the amino acids in the sequence. An entire protein can be sequenced this way, or a sequence fragment can be subjected to database mining to find identity candidates.

The preferred biological source for detection of the biomarkers is serum. However, in other embodiments, the biomarkers are detected in urine and other biological samples.

The biomarkers of this invention are biomolecules. Accordingly, this invention provides these biomolecules in isolated form. The biomarkers can be isolated from biological fluids, such as serum. They can be isolated by any method known in the art, based on both their mass and their binding characteristics. For example, a sample comprising the biomolecules can be subject to chromatographic fractionation, as described herein, and subject to further separation by, e.g., acrylamide gel electrophoresis. Knowledge of the identity of the biomarker also allows their isolation by immunoaffinity chromatography.

2.2. Biomarkers and Modified Forms of a Protein

Proteins frequently exist in a sample in a plurality of different forms. These forms can result from either, or both, of pre- and post-translational modification. Pre-translational modified forms include allelic variants, slice variants and RNA editing forms. Post-translationally modified forms include forms resulting from proteolytic cleavage (e.g., fragments of a parent protein), glycosylation, phosphorylation, lipidation, oxidation, methylation, cysteinylation, sulphonation and acetylation. When detecting or measuring a protein in a sample, the ability to differentiate between different forms of a protein depends upon the nature of the difference and the method used to detect or measure. For example, immunological methods of detection typically cannot distinguish between different forms of a protein that contain the same epitope or epitopes to which the antibody or antibodies are directed. In diagnostic assays, the inability to distinguish different forms of a protein has little impact when the forms detected by the particular method used are equally good biomarkers as any particular form. However, when a particular form (or a subset of particular forms) of a protein is a better biomarker than the collection of modified forms detected together by a particular method, the power of the assay may suffer. In this case, it is useful to employ an assay method that distinguishes between forms of a protein and that specifically detects and measures a desired modified form or forms of the protein. Distinguishing different forms of an analyte or specifically detecting a particular form of an analyte is referred to as “resolving” the analyte.

The collection of analytes detected in an assay and the ability to resolve modified forms of a protein of course depends on the methodology used. For example, an immunoassay using a monoclonal antibody will detect all forms of a protein containing the eptiope and will not distinguish between them. However, a sandwich immunoassay that uses two antibodies directed against different epitopes on a protein will detect all forms of the protein that contain both epitope and will not detect those forms that contain only one of the epitopes. Accordingly this method can be useful when the modified forms differ in a terminal amino acid and one of the antibodies is directed to the terminus of one of these forms.

Preferably, the biospecific capture reagent is bound to a solid phase, such as a bead, a plate, a membrane or a chip. Methods of coupling biomolecules, such as antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations. For example, one can load multiple columns with derivatized beads, each column able to capture a single protein cluster. Alternatively, one can pack a single column with different beads derivatized with capture reagents against a variety of protein clusters, thereby capturing all the analytes in a single place. Accordingly, antibody-derivatized bead-based technologies, such as xMAP technology of Luminex (Austin, Tex.) can be used to detect the protein clusters. However, the biospecific capture reagents must be specifically directed toward the members of a cluster in order to differentiate them.

Mass spectrometry is a particularly powerful resolving methodology because different forms of a protein typically have different masses and can be differentiated by mass spectrometry. One useful methodology combines mass spectrometry with immunoassay. First, a biospecific capture reagent (e.g., an antibody, aptamer or Affibody that recognizes the biomarker and modified forms of it) is used to capture the biomarker of interest. Preferably, the biospecific capture reagent is bound to a solid phase, such as a bead, a plate, a membrane or a chip. After unbound materials are washed away, the captured analytes are detected and/or measured by mass spectrometry. (This method also will also result in the capture of protein interactors that arc bound to the proteins or that arc otherwise recognized by antibodies and that, themselves, can be biomarkers.) Then, the captured proteins can be detected by SELDI mass spectrometry or by eluting the proteins from the capture reagent and detecting the eluted proteins by traditional MALDI, SELDI or any other ionization method for mass spectrometry (e.g., electrospray).

Thus, when reference is made herein to detecting a particular protein or to measuring the amount of a particular protein, it means detecting and measuring the protein with or without resolving modified forms of protein. For example, the step of “measuring Apolipoprotein A-IV precursor” includes measuring Apolipoprotein A-IV precursor by means that do not differentiate between various forms of the protein (e.g., certain immunoassays) as well as by means that differentiate some forms from other forms or that measure a specific form of the protein. In contrast, when it is desired to measure a particular form or forms of a protein, the particular form (or forms) is specified. For example, “measuring M7.065159” or a biomarker of 7.065159 kDa means measuring it in a way that distinguishes it from forms of the protein that do not have the characteristic properties identified in Tables 1 and 2.

### 3. Detection of Biomarkers for Babesia

The biomarkers of this invention can be detected by any suitable method. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Protein biochips are biochips adapted for the capture of polypeptides. Many protein biochips are described in the art. These include, for example, protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK). Examples of such protein biochips arc described in the following patents or published patent applications: U.S. Pat. No. 6,225,047 (Hutchens & Yip); U.S. Pat. No. 6,537,749 (Kuimelis and Wagner); U.S. Pat. No. 6,329,209 (Wagner et al.); PCT International Publication No. WO 00/56934 (Englert et al.); PCT International Publication No. WO 03/048768 (Boutell et al.) and U.S. Pat. No. 5,242,828 (Bergstrom et al.).

3.1. Detection by Mass Spectrometry

In a preferred embodiment, the biomarkers of this invention are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization Mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

3.1.1. SELDI

A preferred mass spectrometric technique for use in the invention is “Surface Enhanced Laser Desorption and Ionization” or “SELDI,” as described, for example, in U.S. Pat. No. 5,719,060 and U.S. Pat. No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and H50 (hydrophobic); SAX-2, Q-10 and LSAX-30 (anion exchange); WCX-2, CM-10 and LWCX-30 (cation exchange); IMAC-3, IMAC-30 and IMAC 40 (metal chelate); and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities that adsorb transition metal ions, such as copper, nickel, zinc, and gallium, by chelation. Preactivated ProteinChip arrays have acyl-imidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. patent application Ser. No. U.S. 2003/0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. patent application Ser. No. US 2003/0218130 A1 (Boschetti et al., “Biochips With Surfaces Coated With Polysaccharide-Based Hydrogels,” Apr. 14, 2003) and U.S. patent application Ser. No. 60/448,467, entitled “Photocrosslinked Hydrogel Surface Coatings” (Huang et al., filed Feb. 21, 2003).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

The biomarkers bound to the substrates are detected in a gas phase ion spectrometer such as a time-of-flight mass spectrometer. The biomarkers are ionized by an ionization source such as a laser, the generated ions arc collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyacetophenone derivatives. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-ethoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 6,124,137 and PCT International Publication No. WO 03/64594 (Kitagawa, “Monomers And Polymers Having Energy Absorbing Moieties Of Use In Desorption/Ionization Of Analytes,” Aug. 7, 2003).

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI arc readily adapted to detecting a biomarker or biomarker profile, pursuant to the present invention.

3.1.2. Other Mass Spectrometry Methods

In another mass spectrometry method, the biomarkers are first captured on a chromatographic resin having chromatographic properties that bind the biomarkers. In the present example, this could include a variety of methods. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI. In yet another method, one could isolate the biomarkers using gel elecrophoresis and detect the biomarkers by MALDI OR SELDI.

3.1.3. Data Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, as part of Ciphergen's ProteinChip® software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

3.1.4. General Protocol for SELDI Detection of Biomarkers for Babesia

A preferred protocol for the detection of the biomarkers of this invention is as follows. The biological sample to be tested, e.g., serum, preferably is subject to pre-fractionation before SELDI analysis. This simplifies the sample and improves sensitivity. A preferred method of pre-fractionation involves contacting the sample with an anion exchange chromatographic material, such as Q HyperD (BioSepra, SA). The bound materials are then subject to stepwise pH elution using buffers at pH 9, pH 7, pH 5 and pH 4. (The fractions in which the biomarkers are eluted also is indicated in Table 1.) Various fractions containing the biomarker are collected.

The sample to be tested (preferably pre-fractionated) is then contacted with an affinity capture probe comprising an cation exchange adsorbent (preferably a WCX ProteinChip array (Ciphergen Biosystems, Inc.)) or an IMAC adsorbent (preferably an IMAC3 ProteinChip array (Ciphergen Biosystems, Inc.)), again as indicated in Table 1. The probe is washed with a buffer that will retain the biomarker while washing away unbound molecules. A suitable wash for each biomarker is the buffer identified in Table 1. The biomarkers are detected by laser desorption/ionization mass spectrometry.

Alternatively, if antibodies that recognize the biomarker are available, these can be attached to the surface of a probe, such as a pre-activated PS10 or PS20 ProteinChip array (Ciphergen Biosystems, Inc.). These antibodies can capture the biomarkers from a sample onto the probe surface. Then the biomarkers can be detected by, e.g., laser desorption/ionization mass spectrometry.

3.2. Detection by Immunoassay

In another embodiment of the invention, the biomarkers of the invention are measured by a method other than mass spectrometry or other than methods that rely on a measurement of the mass of the biomarker. In one such embodiment that does not rely on mass, the biomarkers of this invention are measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays. Nephelometry is an assay done in liquid phase, in which antibodies are in solution. Binding of the antigen to the antibody results in changes in absorbance, which is measured. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### 4. Determination of Subject Babesia Status

4.1. Single Markers

The biomarkers of the invention can be used in diagnostic tests to assess babesia status in a subject, e.g., to diagnose Babesia. The phrase “Babesia status” includes any distinguishable manifestation of the disease, including non-disease. For example, disease status includes, without limitation, the presence or absence of disease (e.g., babesia v. non babesia or Babesia v. other parasitic disease (e.g., African sleeping sickness, Chagas, malaria)), the risk of developing disease, the stage of the disease, the progress of disease (e.g., progress of disease or remission of disease over time) and the effectiveness or response to treatment of disease. The status of the subject may inform the practitioner about what status set is being distinguished. For example, a subject that presents with signs of a parasitic disease could be classed into Babseia v. non-Babesia parasitic disease, while a person exposed to a situation in which Babesia infection is possible and who is presenting with signs of Babesia infection could be classified into Babesia v. non-Babesia. Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that arc predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

The biomarkers of this invention show a statistical difference in different babesia statuses of at least p≦0.05, p≦10⁻², p≦10⁻³, p≦10⁻⁴ or p≦10⁻⁵. Diagnostic tests that use these biomarkers alone or in combination show a sensitivity and specificity of at least 75%, at least 80%, at least 85%, at least 90%, at least 95%, at least 98% and about 100%.

Each biomarker listed in Tables 1, 2 and 3 is differentially present in babesia, and, therefore, each is individually useful in aiding in the determination of babesia status. The method involves, first, measuring the selected biomarker in a subject sample using the methods described herein, e.g., capture on a SELDI biochip followed by detection by mass spectrometry and, second, comparing the measurement with a diagnostic amount or cut-off that distinguishes a positive babesia status from a negative babesia status. The diagnostic amount represents a measured amount of a biomarker above which or below which a subject is classified as having a particular babesia status. For example, if the biomarker is up-regulated compared to normal during babesia, then a measured amount above the diagnostic cutoff provides a diagnosis of babesia. Alternatively, if the biomarker is down-regulated during babesia, then a measured amount below the diagnostic cutoff provides a diagnosis of babesia. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarkers in a statistically significant number of samples from subjects with the different babesia statuses, as was done here, and drawing the cut-off to suit the diagnostician's desired levels of specificity and sensitivity.

4.2. Combinations of Markers

While individual biomarkers are useful diagnostic biomarkers, it has been found that a combination of biomarkers can provide greater predictive value of a particular status than single biomarkers alone. Specifically, the detection of a plurality of biomarkers in a sample can increase the sensitivity and/or specificity of the test. A combination of at least two biomarkers is sometimes referred to as a “biomarker profile” or “biomarker fingerprint.”

4.3. Presence of Babesia

In one embodiment, this invention provides methods for determining the presence or absence of babesia in a subject (status: babesia v. non- babesia). The presence or absence of babesia is determined by measuring the relevant biomarker or biomarkers and then either submitting them to a classification algorithm or comparing them with a reference amount and/or pattern of biomarkers that is associated with the particular risk level.

4.4. Determining Risk of Developing Disease

In one embodiment, this invention provides methods for determining the risk of developing disease in a subject. Biomarker amounts or patterns are characteristic of various risk states, e.g., high, medium or low. The risk of developing a disease is determined by measuring the relevant biomarker or biomarkers and then either submitting them to a classification algorithm or comparing them with a reference amount and/or pattern of biomarkers that is associated with the particular risk level

4.5. Determining Stage of Disease

In one embodiment, this invention provides methods for determining the stage of disease in a subject. Each stage of the disease has a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage.

4.6. Determining Course (Progression/Remission) of Disease

In one embodiment, this invention provides methods for determining the course of disease in a subject. Disease course refers to changes in disease status over time, including disease progression (worsening) and disease regression (improvement). Over time, the amounts or relative amounts (e.g., the pattern) of the biomarkers changes. Therefore, the trend of these markers, either increased or decreased over time toward diseased or non-diseased indicates the course of the disease. Accordingly, this method involves measuring one or more biomarkers in a subject at least two different time points, e.g., a first time and a second time, and comparing the change in amounts, if any. The course of disease is determined based on these comparisons.

4.7. Subject Management

In certain embodiments of the methods of qualifying babesia status, the methods further comprise managing subject treatment based on the status. Such management includes the actions of the physician or clinician subsequent to determining babesia status. For example, if a physician makes a diagnosis of babesia, then a certain regime of treatment, such as prescription or administration of quinine, clindamycin or a combination thereof, might follow. Alternatively, a diagnosis of non- babesia might be followed with further testing to determine a specific disease that might the patient might be suffering from. Also, if the diagnostic test gives an inconclusive result on babesia status, further tests may be called for.

The methods described herein can be used in combination with and other tests and/or methods that are used to qualify babesia status in a subject. For example, in certain aspects, the methods described herein are used to determine whether or not a subject has an increased likelihood of having babesia. These methods can be used in combination with other tests that are useful for either diagnosing babesia in a subject or ruling out other diagnoses.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the presence or absence in a test subject of any the biomarkers of Table 1, 2 or 3 is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

4.8. Determining Therapeutic Efficacy of Pharmaceutical Drug

In another embodiment, this invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of the biomarkers of this invention changes toward a non-disease profile. One can follow the course of the amounts of these biomarkers in the subject during the course of treatment. Accordingly, this method involves measuring one or more biomarkers in a subject receiving drug therapy, and correlating the amounts of the biomarkers with the disease status of the subject. One embodiment of this method involves determining the levels of the biomarkers at least two different time points during a course of drug therapy, e.g., a first time and a second time, and comparing the change in amounts of the biomarkers, if any. For example, the biomarkers can be measured before and after drug administration or at two different time points during drug administration. The effect of therapy is determined based on these comparisons. If a treatment is effective, then the biomarkers will trend toward normal, while if treatment is ineffective, the biomarkers will trend toward disease indications. If a treatment is effective, then the biomarkers will trend toward normal, while if treatment is ineffective, the biomarkers will trend toward disease indications.

### 5. Generation of Classification Algorithms for Qualifying Babesia Status

In some embodiments, data derived from the spectra (e.g., mass spectra or time-of-flight spectra) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes arc described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART - classification and regression trees), artificial neural networks such as back propagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. patent application Ser. No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. patent application Ser. No. 2002 0193950 A1 (Gavin et al., “Method or analyzing mass spectra”), U.S. patent application Ser. No.2003 0004402 A1 (Hitt et al., “Process for discriminating between biological states based on hidden patterns from biological data”), and U.S. patent application Ser. No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for babesia. The classification algorithms, in turn, form the base for diagnostic tests by providing diagnostic values (e.g., cut-off points) for biomarkers used singly or in combination.

### 6. Compositions of Matter

In another aspect, this invention provides compositions of matter based on the biomarkers of this invention.

In one embodiment, this invention provides biomarkers of this invention in purified form. Purified biomarkers have utility as antigens to raise antibodies. Purified biomarkers also have utility as standards in assay procedures. As used herein, a “purified biomarker” is a biomarker that has been isolated from other proteins and peptides, and/or other material from the biological sample in which the biomarker is found. Biomarkers may be purified using any method known in the art, including, but not limited to, mechanical separation (e.g., centrifugation), ammonium sulphate precipitation, dialysis (including size-exclusion dialysis), size-exclusion chromatography, affinity chromatography, anion-exchange chromatography, cation-exchange chromatography, and methal-chelate chromatography. Such methods may be performed at any appropriate scale, for example, in a chromatography column, or on a biochip.

In another embodiment, this invention provides a biospecific capture reagent, optionally in purified form, that specifically binds a biomarker of this invention. In one embodiment, the biospecific capture reagent is an antibody. Such compositions are useful for detecting the biomarker in a detection assay, e.g., for diagnostics.

In another embodiment, this invention provides an article comprising a biospecific capture reagent that binds a biomarker of this invention, wherein the reagent is bound to a solid phase. For example, this invention contemplates a device comprising bead, chip, membrane, monolith or microtiter plate derivatized with the biospecific capture reagent. Such articles are useful in biomarker detection assays.

In another aspect this invention provides a composition comprising a biospecific capture reagent, such as an antibody, bound to a biomarker of this invention, the composition optionally being in purified form. Such compositions are useful for purifying the biomarker or in assays for detecting the biomarker.

In another embodiment, this invention provides an article comprising a solid substrate to which is attached an adsorbent, e.g., a chromatographic adsorbent or a biospecific capture reagent, to which is further bound a biomarker of this invention. In one embodiment, the article is a biochip or a probe for mass spectrometry, e.g., a SELDI probe. Such articles are useful for purifying the biomarker or detecting the biomarker.

7. Kits for Detection of Biomarkers for Babesia

In another aspect, the present invention provides kits for qualifying babesia status, which kits are used to detect biomarkers according to the invention. In one embodiment, the kit comprises a solid support, such as a chip, a microtiter plate or a bead or resin having a capture reagent attached thereon, wherein the capture reagent binds a biomarker of the invention. Thus, for example, the kits of the present invention can comprise mass spectrometry probes for SELDI, such as ProteinChip® arrays. In the case of biospecfic capture reagents, the kit can comprise a solid support with a reactive surface, and a container comprising the biospecific capture reagent.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for suitable operational parameters in the form of a label or separate insert. For example, the instructions may inform a consumer about how to collect the sample, how to wash the probe or the particular biomarkers to be detected.

In yet another embodiment, the kit can comprise one or more containers with biomarker samples, to be used as standard(s) for calibration.

8. Use of Biomarkers for B abesia in Screening Assays and Methods of Treating Babesia

The methods of the present invention have other applications as well. For example, the biomarkers can be used to screen for compounds that modulate the expression of the biomarkers in vitro or in vivo, which compounds in turn may be useful in treating or preventing babesia in patients. In another example, the biomarkers can be used to monitor the response to treatments for babesia. In yet another example, the biomarkers can be used in heredity studies to determine if the subject is at risk for developing babesia.

Thus, for example, the kits of this invention could include a solid substrate having a hydrophobic function, such as a protein biochip (e.g., a Ciphergen H50 ProteinChip array, e.g., ProteinChip array) and a sodium acetate buffer for washing the substrate, as well as instructions providing a protocol to measure the biomarkers of this invention on the chip and to use these measurements to diagnose babesia.

Compounds suitable for therapeutic testing may be screened initially by identifying compounds which interact with one or more biomarkers listed in Table 1, 2 or 3. By way of example, screening might include recombinantly expressing a biomarker listed in Table 1, 2 or 3, purifying the biomarker, and affixing the biomarker to a substrate. Test compounds would then be contacted with the substrate, typically in aqueous conditions, and interactions between the test compound and the biomarker are measured, for example, by measuring elution rates as a function of salt concentration. Certain proteins may recognize and cleave one or more biomarkers of Table 1, 2 or 3, in which case the proteins may be detected by monitoring the digestion of one or more biomarkers in a standard assay, e.g., by gel electrophoresis of the proteins.

In a related embodiment, the ability of a test compound to inhibit the activity of one or more of the biomarkers of Table 1, 2 or 3 may be measured. One of skill in the art will recognize that the techniques used to measure the activity of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker may be determined by measuring the rates of catalysis in the presence or absence of the test compounds. The ability of a test compound to interfere with a non-enzymatic (e.g. structural) function or activity of one of the biomarkers of Table 1, 2 or 3 may also be measured. For example, the self-assembly of a multi-protein complex which includes one of the biomarkers of Table 1, 2 or 3 may be monitored by spectroscopy in the presence or absence of a test compound. Alternatively, if the biomarker is a non-enzymatic enhancer of transcription, test compounds which interfere with the ability of the biomarker to enhance transcription may be identified by measuring the levels of biomarker-dependent transcription in vivo or in vitro in the presence and absence of the test compound.

Test compounds capable of modulating the activity of any of the biomarkers of Table 1, 2 or 3 may be administered to patients who are suffering from or are at risk of developing babesia. For example, the administration of a test compound which increases the activity of a particular biomarker may decrease the risk of babesia in a patient if the activity of the particular biomarker in vivo prevents the accumulation of proteins for babesia. Conversely, the administration of a test compound which decreases the activity of a particular biomarker may decrease the risk of babesia in a patient if the increased activity of the biomarker is responsible, at least in part, for the onset of babesia.

In an additional aspect, the invention provides a method for identifying compounds useful for the treatment of disorders such as babesia which are associated with increased levels of modified forms of the biomarkers in Table 1, 2 or 3. For example, in one embodiment, cell extracts or expression libraries may be screened for compounds which catalyze the cleavage of a full-length biomarker to form truncated forms of the biomarker. In one embodiment of such a screening assay, cleavage of the biomarker may be detected by attaching a fluorophore to the biomarker which remains quenched when the biomarker is uncleaved but which fluoresces when the protein is cleaved. Alternatively, a version of full-length biomarker modified so as to render the amide bond between amino acids x and y uncleavable may be used to selectively bind or “trap” the cellular protesase which cleaves full-length biomarker at that site in vivo. Methods for screening and identifying proteases and their targets are well-documented in the scientific literature, e.g., in Lopez-Ottin et al. (Nature Reviews, 3:509-519 (2002)).

In yet another embodiment, the invention provides a method for treating or reducing the progression or likelihood of a disease, e.g., babesia, which is associated with the increased levels of a truncated biomarker. For example, after one or more proteins have been identified which cleave the full-length biomarker, combinatorial libraries may be screened for compounds which inhibit the cleavage activity of the identified proteins. Methods of screening chemical libraries for such compounds are well-known in art. See, e.g., Lopez-Otin et al. (2002). Alternatively, inhibitory compounds may be intelligently designed based on the structure of the biomarker.

At the clinical level, screening a test compound includes obtaining samples from test subjects before and after the subjects have been exposed to a test compound. The levels in the samples of one or more of the biomarkers listed in Table 1, 2 or 3 may be measured and analyzed to determine whether the levels of the biomarkers change after exposure to a test compound. The samples may be analyzed by mass spectrometry, as described herein, or the samples may be analyzed by any appropriate means known to one of skill in the art. For example, the levels of one or more of the biomarkers listed in Table 1, 2 or 3 may be measured directly by Western blot using radio- or fluorescently-labeled antibodies which specifically bind to the biomarkers. Alternatively, changes in the levels of mRNA encoding the one or more biomarkers may be measured and correlated with the administration of a given test compound to a subject. In a further embodiment, the changes in the level of expression of one or more of the biomarkers may be measured using in vitro methods and materials. For example, human tissue cultured cells which express, or are capable of expressing, one or more of the biomarkers of Table 1, 2 or 3 may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with babesia, test compounds will be screened for their ability to slow or stop the progression of the disease.

### 9. EXAMPLES

### 9.1. Example 1

### Discovery of Biomarkers for Babesia

It is understood that the examples and embodiments described herein are for illustrative purposes only and that various modifications or changes in light thereof will be suggested to persons skilled -in the art and are to be included within the spirit and purview of this application and scope of the appended claims. All publications, patents, and patent applications cited herein are hereby incorporated by reference in their entirety for all purposes.

Two, complimentary approaches to identifying potential biomarkers for the diagnosis of human babesiosis have been taken: 1) SELDI-based and 2) 2-D gels (DIGE technology). Based on estimated molecular weight, there is an overlap of at least 5 biomarkers identified by both approaches (MWs 22, 28, 33, 44 and 146 kDa).

### SELDI Analysis

A total of 20 positive and 20 negative for babesia and positive samples from others protozoan parasites were examined: African sleeping sickness (n=10), Chagas disease (n=10) and malaria (n=10).

The serum samples fractionation was done using 96 well filtration plate containing Q Ceramic HyperD F according to the manufacturer's instructions (Ciphergen, Fremont, Calif., Cat. # K100-0007) on a BioMek 2000 (Beckman Coulter). Briefly, 200 μl of rehydration buffer (50 mM Tris-HCl, pH 9) was added 2 times to each well and equilibrated 3 times with U1 buffer [1 M urea, 2% (w/v) CHAPS, 50 mM Tris-HC1, pH 9]. Twenty microliter of each serum were mixed with 30 μl of U9 buffer [9 M urea, 2% (w/v) CHAPS, 50 mM Tris-HCl, pH 9] in a 96 wells plate v-bottom for 20 min. The sample was then diluted with 50 μl of U1 buffer. One hundred microliters of the diluted serum sample were applied to each well, incubated and mixed on MicroMix (Beckman Coulter) for 30 min. The flow-through was collected by vacuum filtration into v-bottom microplates. The anion-exchange resin was incubated with an additional 100 μl of Tris-HCl buffer [50 mM Tris-HCl, pH 9, 0.1% (w/v) OGP] for 10 min at room temperature with shaking. The wash was collected by vacuum filtration. This procedure was repeated two times with 100 μl each of appropriate buffers with decreasing pH (pH 7, 5, 4, 3 and organic). The final wash was performed with an organic wash buffer containing 33% (v/v) isopropanol and 16.7% (v/v) acetonitrile in 0.1% trifluoroacetic acid (TFA). Fractionated samples were stored at −80 C. until analysis.

The following chip binding protocol was followed and the samples were processed using an IMAC-3 ProteinChip Array according to the protocol below:

### Chip Binding Protocol

### Weak Cation Exchange (WCX2) ProteinChip Array

### Materials:

Bioprocessor
WCX-2 chip
Vortex
CM low stringency buffer
Deionized water
EAM solution
   1. Assemble the WCX-2 protein chip in the bioprocessor.
   2. Add 150 ul of CM low stringency buffer to each well.
   3. Vortex for 5 minutes (speed 100 rpm) at room temperature.
   4. Remove the buffer from the wells.
   5. Repeat steps 2 to 3 for a total of 2 washes.
   6. Add 90 ul of CM low stringency buffer to each well.
   7. Add 10 ul of sample (fractions) to each well.
   8. Vortex for 30 minutes (speed 100 rpm) at room temperature.
   9. Remove the samples from the wells.
   10. Wash each well with 150 ul CM low stringency buffer.
   11. Vortex for 5 minutes (100 rpm).
   12. Repeat twice for a total of three buffer washes.
   13. Remove the washing buffer from the wells and rinse each well with deionized water.
   14. Drain the wells and remove the chip from the bioprocessor.
   15. Allow the chip to air dry.
   16. Apply 0.5-1 ul of EAM solution per spot twice.
   17. Allow to air dry after each application.
   18. Analyze the chip.
      Processing Samples using an IMAC-3 ProteinChip Array

### Material:

Bioprocessors
IMAC Chips
Pap Pen
Votex (VWR VX-2500 Multitube Vortexer)

### IMAC3 Chip Buffer:

A) Binding Buffer: 100 mM Sodium Phosphate+0.5M NaCl pH 7.0+0.1% Triton X 20
B) Charging Buffer (Copper): 100 nM CUSO₄+0.1% Triton X 20
C) Neutralizing Buffer: 100 mM NaAcetate pH 4.0+0.1% Triton X 20
   1. Place Chip in bioprocessor
   2. Load IMAC chips with copper: Apply 50 μl/well of 100 mM CuSO₄
   3. Vortex 5 min (speed 100 rpm) at room temperature
   4. Remove CuSO₄
   5. Wash with water 120 μl/well
   6. Vortex 5 min (speed 100 rpm)
   7. Neutralize chips: Add 50 μl/well of 100 mM NaAcetate pH 4.0
   8. Remove solution
   9. Wash with water 120 μl/well
   10. Vortex 5 min (speed 100 rpm)
   11. Repeat steps 9 & 10 a further two times
   12. Equilibrate Chips: Add 120 μl Binding Buffer (PBS/0.5 M NaCl, pH 7.5)
   13. Vortex 5 min (100 rpm)
   14. Bind fractions to chips: Discard waste and add 80 μl Binding Buffer and 20 μl of fractions (containing samples)
   15. Vortex 45-60 min (100 rpm)
   16. Discard and wash (PBS/0.5M NaCl, 150 μl/well)
   17. Vortex 5 min (100 rpm)
   18. Repeat steps 16 & 17 a further two times
   19. Rinse chip with dH₂O (150 μl/well)
   20. Add Matrix: Remove bioproceesor top and gasket
   21. Rinse the Chips quickly with dH₂O
   22. Dry chips
   23. Circle spots with PAP pen
   24. Add 0.5 μl SPA to Chips two times (air dry the spots between addition)
      Ciphergen normally supplies EAM as 5 mg of dried powder in a tube.
      Add 100 μl of 100% Acetonitrile (final concentration 50% ACN)+50 μl 2%
      Trifluoroacetic acid (final conc. 0.5% TFA)+50 μl dH₂O.
      Vortex 1 min (high speed) and leave it in the bunch for 5 min
      Spin 2 min at high speed to pellet any particulates
   25. Dry
   26. Read within 1 hour

### Data Acquisition

The ProteinChip Arrays were analyzed in the ProteinChip Biology System Reader (Model PBS IIc, Ciphergen Biosystems) with an autoloader. The spectra were collected using two different laser intensities (low and high) for each fraction (pH).

The data were analyzed by ProteinChip Software version 3.0 (Ciphergen Biosystems), CiphergenExpress version 2.1 and Biomarkers pattern Software version 2.2. All spectra were subjected to mass calibration based on the settings used to collect the data, baseline subtraction and noise at 2,000 Da for the low energy and 10,000 Da for the high energy. All data were normalized by total ion current normalization for low intensity (2-100 kDa) and high intensity (10-200 kDa) using an external coefficient of 0.2. Signal-to-noise ratio (S/N) was set at 3, with a minimum peak threshold at 10%, a cluster mass window at 0.3% for the first pass and S/N at 2 for the same settings for the second pass except the cluster mass set at 2%.

### Analysis Using 2D-DIGE and MALDI/TOF MS

A total of 33 sera samples (16 controls, 17 babesia-infected) were tested for biomarker discovery using differential gel electrophoresis followed by protein identification by matrix-assisted laser desorption/ionization mass spectrometry (DIGE and MALDI-TOFMS). In brief, protein was isolated from each individual serum specimen and then combined to generate 4 separate sub-pools for each sera type (i.e., n=4 individuals for each babesia and control sub-pool). Each sub-pool was then labeled with either Cy3 or Cy5 fluorescent dye, combined with a sub-pool from the opposing group (and stained with the other dye) and run by DIGE. (i.e, There were 4 gels -run in total.) Differences in protein levels between each group were then determined following scanning and image analysis (Decyder software, GE Heathcare). A sample containing a mix of all specimens was labeled with Cy2 dye and run on each gel to facilitate gel-to-gel comparisons. Differences in protein levels between control and babesia sub-pools were further validated using DIGE runs containing a pool of all control specimens versus a pool of all babesia-infected sera (i.e., two gels of the same total pools with a dye swap for disease versus control). Biomarkers for babesiosis were identified based on identical increase/decrease trends observed in the replicate sub-pools (4 gels) and in the total babesia verses total control pools (2 gels). Visual inspection of all interesting protein spots was also used to validate the ratios determined by the Decyder program for all gels. Protein spots that met the stated criteria were picked from the gel, digested with trypsin, and identified by either MALDI-TOF or LC-MS/MS. A total of 37 protein spots corresponding to 21 unique proteins were determined. to either increase/decrease with babesia-infection in human sera. Many of these potential biomarkers are associated with an increase in the immune/inflammatory response (alpha 1-antitrypsin, immunoglobulins, complement component 4A, compliment factor B, and CD5-like antigen) or indicative of accelerated hemolysis with infection (hemoglobin B chain and haptoglobin).

Each serum sample contained approx. 40 μL of serum. A key to the specimens is as follows:

| Babesia | negative | positive |
|---|---|---|
| 2004 | 12-22 | 1-8, 10, 11 |
| 2005 | 1, 6-8, 10-12 | 2-5, 9 |

Protein was isolated independently for each serum sample by a methanol/chloroform procedure and the concentrations in each were determined by a Bradford Protein Assay. All samples were diluted with the appropriate buffer to yield a final concentration of 5 mg/mL protein per specimen. Equal volumes from 4 specimens per sera group were combined to create 8 sub-pools: 4 sub-pools for control and 4 sub-pools for babesia-infected serum. The following table lists the individual samples that were combined to generate each sub-pool.

| Serum Type: | Control | Babesia |
|---|---|---|
| Sub-pool #1 | 2MULA, 3GESP, 4GLES, | 1AGRR, 6MELD, 7DUGN, |
| | 5DUGG | 8MCEK |
| Sub-pool #2 | 12-14, 9AADW | 10SCHD, 11MUER, 6, 7 |
| Sub-pool #3 | 15-18 | 1-4 |
| Sub-pool #4 | 19-22 | 5, 8-11 |

An “all-samples pool” was created by combining equal volumes from the above sub-pools into a single mix to serve as a control on all DIGE gels. Pools containing all control samples and all disease samples were also made for each serum type by combining equal volumes from each of the four sub-pools from control or babesia sera.

### Sample Labeling

Before labeling, the protein concentrations for each sub-pool and pool were again determined by the Bradford Protein Assay to make sure equivalent levels of protein were employed for DIGE analysis. Protein (50 μg) was then labeled from control and babesia-infected sera with either Cy3 or Cy5 fluorescent dye and 50 μg protein from the all-samples pool was labeled with Cy2. The Cy3/Cy5 labeling of control/babesia sera was alternated for each gel to avoid any bias that might arise from the labeling chemistry of a particular dye to specific protein. Control sub-pool #1 was compared to babesia sub-pool #1, control sub-pool #2 was compared to babesia sub-pool #2, and so on, while the total control pool and the total babesia pool were compared against each other. (Duplicate experiments were performed with a dye swap.) When the gel was to be used to determine protein ID's, additional unlabeled protein (425 μg) from both control and babesia sera was spiked in following the labeling reaction. The Cy3-, Cy5- and Cy2-labeled specimens were then combined, reduced with HED, mixed with appropriate pharmalytes, colored with bromophenol blue, and used immediately for DIGE.

### DIGE Analysis

Strip rehydration and focusing: Labeled control/babesia/all sample mix was applied to Amersham Immobiline DryStrips (pH 3-10, 24 cm) for the purpose of separating proteins based on charge. The strips were rehydrated with protein samples overnight, then run for ≠66,000 Vhr on an IPGphor Isoelectric Focusing Unit (Amersham Biosciences). Following focusing, the strips were treated with a reduction solution (DTT in a SDS-equilibration buffer) and an alkylation solution (iodoacetamide in SDS-equilibration buffer). Following reduction and alkylation the strips were immediately transferred to analytical (all sub-pools) or preparative (total control/babesia pools) gels for protein size separation.

Analytical gels (control/babesia sub-pools): Immobiline DryStrips containing the control vs. babesia sub-pools were placed at the top of 4, 8-16% acrylamide gradient gels (Jule Biotechnologies) and run at 1.5 V per gel overnight. The protein concentration loaded for analytical gels was sufficient to determine differences in protein levels between two samples, but not enough to obtain protein ID's. Once the dye front for each gel reached the bottom of the glass plate, the gels were removed and either scanned immediately or fixed (7.5% acetic acid, 30% methanol) washed, and stored overnight (H₂O, 4° C.) then scanned the next day.

Preparative gels (total control/babesia pools): The protocol for running preparative gels is identical to that described above except that 1 mg of total protein is used instead of 0.150 mg. This facilitates protein identification from the picked spots. Both the total control vs. total babesia gels (i.e., this includes a dye-swap sample) were run on 8-16% acrylamide gradient preparative gels.

### Scanning Fluorescently Labeled Gels

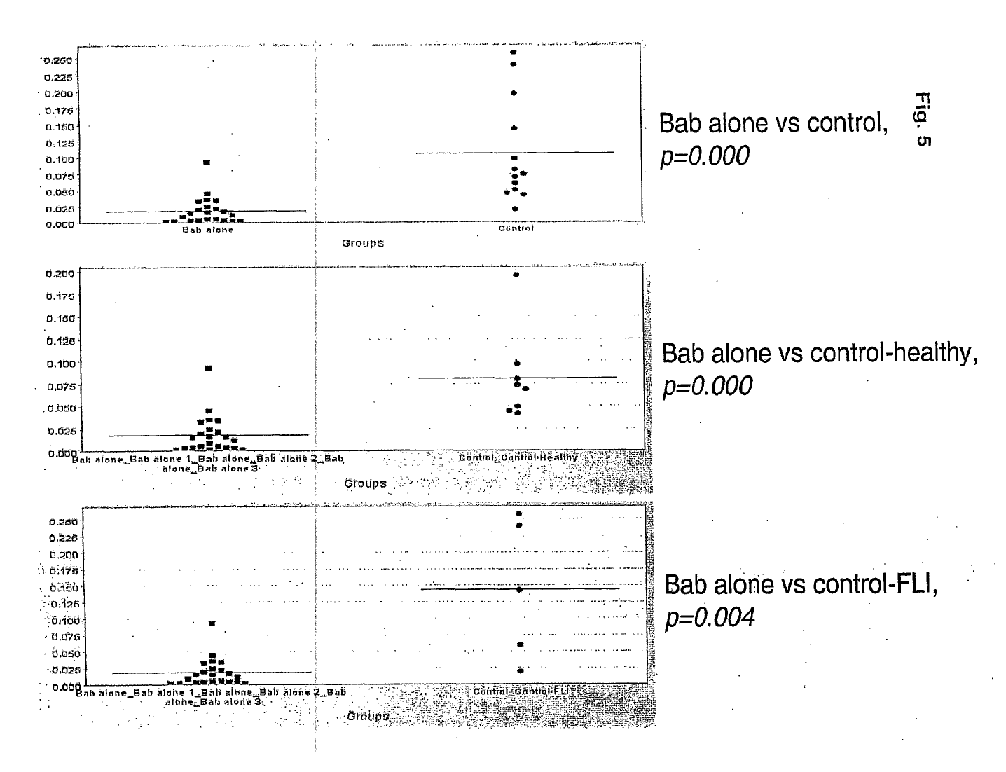

Gels were scanned on a Typhoon 9400 Scanner (Amersham). Emissions from the three different fluorescent dyes (Cy3, Cy5, and Cy2) were measured on separate channels using different filters for wavelength and band pass. This allows for relative protein levels of three different samples, which in our case is control/babesia/combination of all samples, to be measured on the same gel. Furthermore, it is possible to adjust the voltage levels for each dye. Typically, laser intensity was adjusted such that the signal measured for any protein spot was below saturation levels. (See section on the data analysis of preparative gels). The Typhoon Scanner software designates colors for the three fluorescent dyes: Cy3=green, Cy5=red, and Cy2=blue. Images created from scanned gels show a yellow color for protein spots that do not change in levels between the two conditions while spots with a green or red color indicate an increase/decrease in protein levels across the two conditions. (In our case this is control vs. babesia serum samples.) An image of one of the scanned preparative gels is included as FIG. 5.

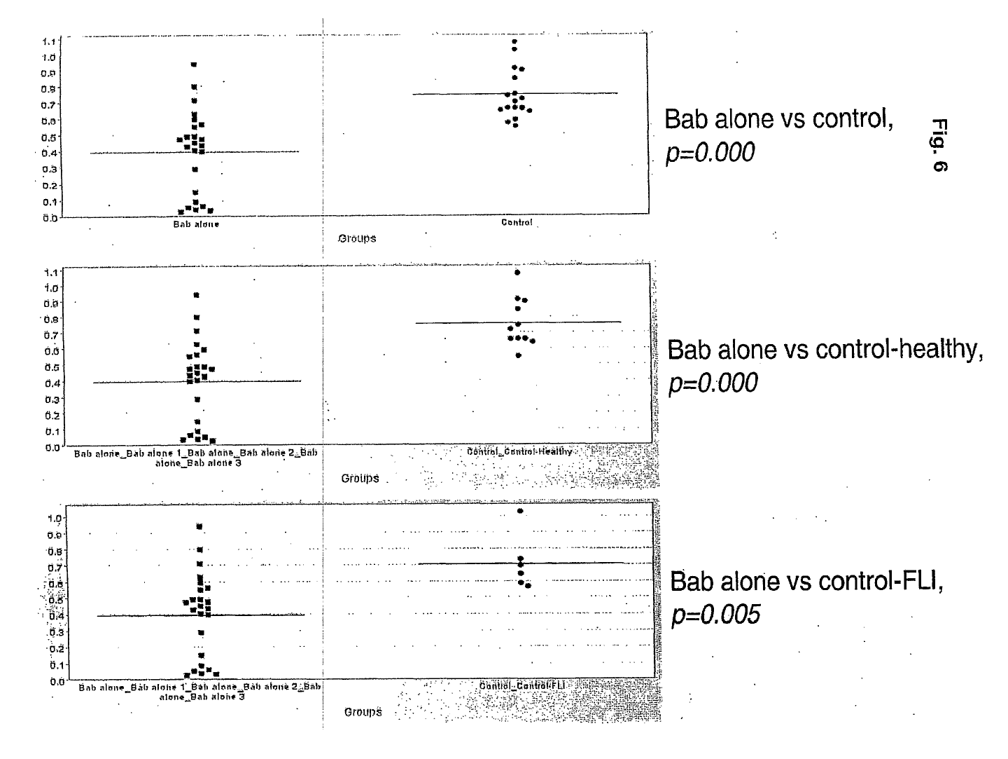

Images of scanned gels were imported into Decyder (Amersham) for differential protein analysis. The Decyder software package was specifically designed for the DIGE technology and allows for functions including: spot detection and quantification, viewing of spot data (image, 3-D, table and histogram views), comparing spot data from multiple gels (average ratio, T-test, ANOVA, etc. . . ) and for creating pick lists. An image of the scanned gel in the previous section is given as FIG. 6 following spot detection by the Decyder software. As with the previous gel picture, spots outlined in yellow show no change in protein levels across control vs. babesia sera, while red outlined spots increase with babesia (>1.5 fold) and green spots decrease (<−1.5 fold) with the disease.

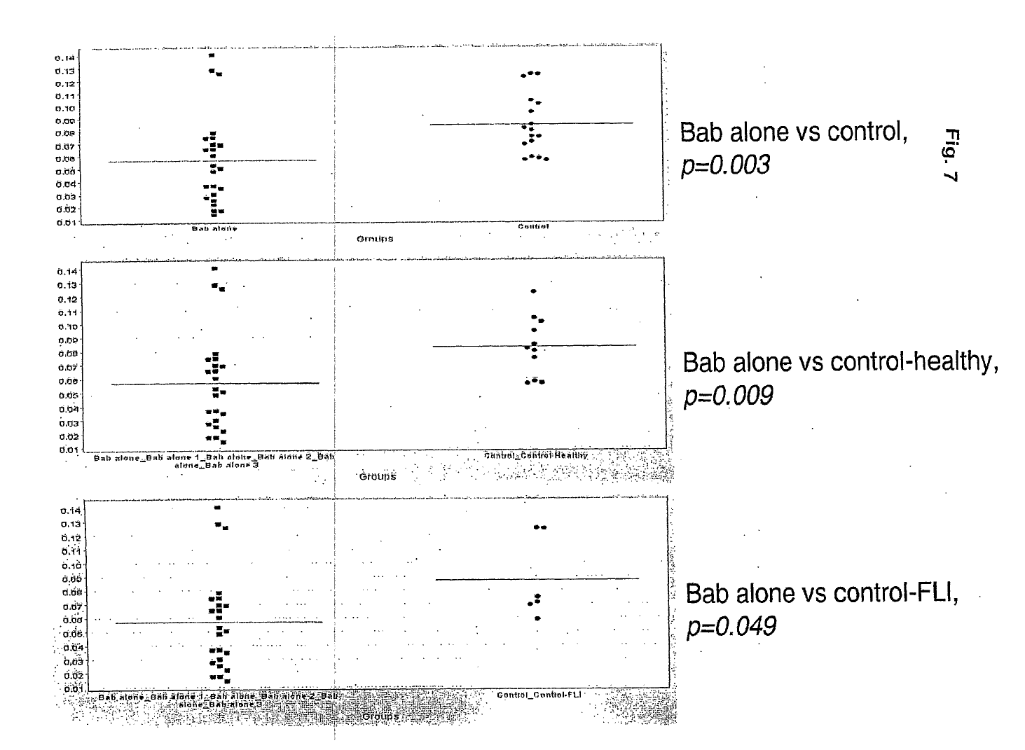

Data analysis of analytical gels. The four control vs. babesia sub-pool gels were processed and the spots detected and matched to determine common differentially expressed proteins. Of 728 spots matched between the four gels, 42 spots produced a T-test p-value of less than 0.05 for control vs. babesia. An example of one of these spots is given below as FIGS. 7A-C showing its image in both control and babesia channels, a 3-D representation of spot volume, and the log₂ expression on all four sub-pool gels.

Data analysis of preparative gels. Two preparative gels comparing all control samples vs. all babesia-infected sample were run. Each gel contained the same samples except that the Cy3/Cy5 labeling of total control/total babesia was switched for each gel.

Protein Identification Using MALDI/TOF and/or LC/MS

Spots determined to be potential biomarkers for babesia were picked from one of the preparative gels using an Ettan Spot Picker (Amersham). The gel plugs were then placed in a 96-well plate and proteins were extracted and digested overnight with trypsin using an Ettan Digester (Amersham). The resulting peptide mixture was then spotted on a MALDI plate and analyzed using a DE-STR MALDI-TOF mass spectrometer (Applied Biosystems). The peptide fingerprint for each spot was compared against the NCBI database (contains all organisms) using Mascot Daemon software (Matrix Science Ltd.). A positive match for protein identification was based on the number of peptides matched to a particular protein, the protein coverage of the matched peptides, and the error in the observed peptide masses as compared to the theoretical masses. If a match couldn't be obtained with a high degree of confidence from the first preparative gel, the same spot was picked from the second preparative gel and processed as before using the same MALDI protocol. If the protein could still not be identified after the second attempt with MALDI, the peptide mixture was then subjected to LC-MS/MS analysis for peptide sequencing. The results from LC-MS/MS were again compared against the NCBI database using predetermined criteria for correct protein identification.

### Results

To determine potential biomarkers for babesia, increasing or decreasing trends across all gels were identified. At medium to high abundance, DIGE is able to differentiate a 20% change in protein levels between labeled samples. a>1.2 fold change in expression (control vs. babesia) was used as the inclusion limit for biomarkers of babesiosis. This>1.2 fold change was required to be observed in the majority of the analytic gel comparisons (3 out of 4 gels) and/or in both preparative gels. Protein spots that met the above conditions were then inspected visually to confirm the identity of the spot on each gel, to provide additional validation on the determined Decyder ratios, and to get an approximation of MW and pI for the spot to aid in protein identification. More spots were identified in the over-exposed preparative gels than in the analytical gels and therefore some changes in spot intensity observed in the preparative gels were not detected in some/all of the analytical gels.

In Table 2 are 37 spots corresponding to roughly 21 unique proteins that are biomarkers for babesiosis. The number of distinct spots identified for each particular protein is given in parentheses. Values in the table are the average fold change (babesia levels÷control levels) across the two preparative gels (total pools) and the four analytical gels (sub-pools)±SD for each unique protein.

A difference in the DIGE experiments is an increase in alpha 1-antitrypsin (AAT) levels with babesiosis. Although only 4 AAT spots are listed in Table 2, there are more spots attributable to this protein around ˜45 kDa and pI of ˜5.4 on each gel. AAT is an acute phase protein and it has been reported that levels can increase up to 4 fold during inflammation (1). Consistent with its role in the inflammatory process, our group has observed increases in AAT levels for a variety of different pathologies and in multiple tissue-types. Conversely, the decrease in the chain A component of AAT may indicate increased stabilization of the entire protein and therefore cleaved products from AAT may be reduced as a result.

An increase in hemoglobin and a decrease in haptoglobin levels maybe indicative of hemolysis associated with the pathology of babesia infection. Both of these proteins had multiple hits in our screen, each with the same increasing/decreasing patterns for each separate spot. An increase in hemoglobin is consistent with the babesia-mediated lysis of erythrocytes following infection. Haptoblobin functions in the cellular defense response by binding to and eliminating free hemoglobin in the blood, thereby canceling its toxic effects (increased ROS production, promoting bacterial growth, etc.) in the body. Haptoglobin is removed from the blood along with the bound hemoglobin, which may explain the decrease in the haptoglobin plasma concentration observed in patients with accclerated hemolysis.

Many of the proteins isolated from the DIGE experiments were associated with the immune response and as expected, these immune-related proteins are present at higher levels in babesia infection. Immunoglobins heavy constant alpha 1 (IGHAL) and M heavy chain (IgM) were increased ca. 1.5-2 fold in babesiosis, while the gamma-1 heavy chain constant region (IGHG1) increased almost 4 fold. Two other proteins, complement factor B (CF) and complement component 4A (C4A), are associated with complementation activation of the immune response. The genes that encode these two proteins are both localized to the major histocompatibility complex (MHC) class TTI region on chromosome 6 (6p21.3). Levels for CD5 antigen-like (CD5L), a protein involved in apoptosis and the cellular defense response, increased ˜1.6 fold with disease. CD5L has been shown to associate with IgM (3), another protein that was isolated in this screen. One immune-related protein, apolipoprotein A-IV precursor (APOA4), had lower levels in the babesia-infected sera as compared to the control (˜2 fold difference in the preparative gels). Visual inspection of the gels showed that the spot corresponding to APOA4 had the clearest difference in levels between the two types of sera from the proteins given in Table 1. The precise function of APOA4 is not known, but it is believed to be involved in lipid metabolism (Gao J et al., J Biol Chem. 2005;280(13):12559-66) and the anti-inflammatory response (Vowinkel et al. J Clin. Invest 2004;1 14(2):260-9). This protein could be a target for destruction by the babesia parasite, or that the host itself decreases the levels of this anti-inflammatory protein in order to combat the infection.

Tables A-P below show the results of a biomarker discovery study. Biomarkers that show a statistical difference in different babesia statuses of at least p≦0.05 are provided in Tables 1 and 2. The biomarkers presented in these tables can be used in all aspects of the present invention. F1CSL and F1CSH refers to Fraction 1, WCX2, SPA, Low or High intensity; F1ISL and F1ISH refer to Fraction 1, IMAC, SPA, Low or High intensity; F3CSL and F3CSH refer to Fraction 3, WCX2, SPA, Low or High intensity; F5CSL or F5CSH refer to Fraction 5, WCX2, SPA, Low or High intensity; F5ISL and F5ISH refer to Fraction 5, 1MAC, SPA, Low or High intensity; F6CSL and F6CSH refer to Fraction 6, WCX2, SPA, Low or High intensity; and F6ISL and F6ISH refer to Fraction 6, IMAC, SPA, Low or High intensity.

TABLE A
Biomarkers identified in F1CSL

| | | | | | | Babesia vs. | | | | | | |
|---|---|---|---|---|---|---|---|---|---|---|---|---|
| Babesia | | | | Babesia vs. | | Babesia | | Healthy vs. | | | | |
| vs. Non- | | Babesia vs. | | Flu-like | | with/Lyme | | Flu-like | | Babesia 1 | | |
| babesia | | Healthy | | symptoms | | Disease | | symptoms | | vs. Babesia 2 | | M/ [symbol] |
| P | | P | | P | | P | | P | | P | | (av [symbol] |
| value | ROC | value | ROC | value | ROC | value | ROC | value | ROC | value | ROC | D [symbol] |
| 0.005 | 0.234 | 0.024 | 0.263 | 0.027 | 0.232 | 0.572 | 0.563 | 0.482 | 0.583 | 0.897 | 0.529 | 2 [symbol] |
| 0.194 | 0.357 | 0.013 | 0.239 | 0.282 | 0.627 | 0.785 | 0.514 | 0.027 | 0.182 | 0.651 | 0.558 | [symbol] |
| 0.024 | 0.705 | 0.034 | 0.706 | 0.178 | 0.696 | 0.082 | 0.312 | 0.366 | 0.667 | 0.439 | 0.596 | [symbol] |
| 0.000 | 0.868 | 0.000 | 0.872 | 0.021 | 0.768 | 0.232 | 0.370 | 0.366 | 0.629 | 0.699 | 0.400 | [symbol] |
| 0.003 | 0.786 | 0.005 | 0.800 | 0.095 | 0.696 | 0.034 | 0.254 | 0.763 | 0.583 | 0.897 | 0.496 | [symbol] |
| 0.001 | 0.807 | 0.001 | 0.848 | 0.085 | 0.699 | 0.572 | 0.447 | 0.315 | 0.674 | 0.439 | 0.625 | [symbol] |
| 0.092 | 0.684 | 0.246 | 0.634 | 0.106 | 0.696 | 0.057 | 0.254 | 0.366 | 0.409 | 0.561 | 0.558 | [symbol] |
| 0.005 | 0.766 | 0.031 | 0.753 | 0.018 | 0.804 | 0.107 | 0.312 | 0.132 | 0.265 | 0.017 | 0.167 | 3 [symbol] |
| 0.057 | 0.643 | 0.024 | 0.753 | 0.667 | 0.587 | 0.005 | 0.196 | 0.070 | 0.720 | 1.000 | 0.467 | 3 [symbol] |
| 0.538 | 0.459 | 0.450 | 0.615 | 0.021 | 0.192 | 0.107 | 0.312 | 0.035 | 0.765 | 0.897 | 0.525 | 3 [symbol] |
| 0.026 | 0.725 | 0.028 | 0.729 | 0.236 | 0.659 | 0.005 | 0.196 | 0.688 | 0.538 | 0.478 | 0.429 | 3 [symbol] |
| 0.042 | 0.684 | 0.014 | 0.777 | 0.667 | 0.554 | 0.014 | 0.196 | 0.108 | 0.720 | 0.561 | 0.429 | 3 [symbol] |
| 0.024 | 0.725 | 0.049 | 0.682 | 0.118 | 0.696 | 0.249 | 0.389 | 0.841 | 0.538 | 0.796 | 0.529 | 3 [symbol] |
| 0.011 | 0.275 | 0.026 | 0.259 | 0.085 | 0.297 | 0.173 | 0.630 | 0.688 | 0.409 | 0.561 | 0.396 | 3 [symbol] |
| 0.019 | 0.316 | 0.004 | 0.176 | 0.706 | 0.446 | 0.098 | 0.698 | 0.012 | 0.136 | 0.561 | 0.429 | 3 [symbol] |
| 0.019 | 0.295 | 0.003 | 0.172 | 0.829 | 0.478 | 0.148 | 0.650 | 0.027 | 0.182 | 0.333 | 0.367 | 3 [symbol] |
| 0.116 | 0.643 | 0.038 | 0.706 | 0.957 | 0.518 | 0.516 | 0.370 | 0.088 | 0.758 | 1.000 | 0.496 | 4 [symbol] |
| 0.006 | 0.766 | 0.002 | 0.824 | 0.333 | 0.623 | 0.068 | 0.312 | 0.228 | 0.667 | 0.606 | 0.492 | 4 [symbol] |
| 0.044 | 0.336 | 0.019 | 0.263 | 0.590 | 0.406 | 0.516 | 0.601 | 0.191 | 0.318 | 1.000 | 0.500 | 4 [symbol] |
| 0.005 | 0.725 | 0.001 | 0.848 | 0.389 | 0.623 | 0.034 | 0.225 | 0.016 | 0.856 | 0.606 | 0.596 | 4 [symbol] |
| 0.026 | 0.725 | 0.080 | 0.682 | 0.067 | 0.732 | 0.009 | 0.225 | 0.615 | 0.409 | 0.606 | 0.562 | 4 [symbol] |
| 0.014 | 0.254 | 0.018 | 0.263 | 0.178 | 0.304 | 0.572 | 0.572 | 0.920 | 0.485 | 0.651 | 0.462 | 4 [symbol] |
| 0.044 | 0.684 | 0.013 | 0.777 | 0.747 | 0.551 | 0.082 | 0.283 | 0.044 | 0.803 | 0.366 | 0.562 | 4 [symbol] |
| 0.010 | 0.746 | 0.016 | 0.753 | 0.118 | 0.696 | 0.068 | 0.312 | 0.763 | 0.576 | 0.651 | 0.596 | 4 [symbol] |
| 0.018 | 0.705 | 0.004 | 0.800 | 0.628 | 0.522 | 0.543 | 0.457 | 0.191 | 0.667 | 0.366 | 0.629 | 5 [symbol] |
| 0.004 | 0.705 | 0.002 | 0.800 | 0.236 | 0.663 | 0.753 | 0.457 | 0.482 | 0.576 | 0.846 | 0.496 | 5 [symbol] |
| 0.245 | 0.398 | 0.080 | 0.287 | 0.788 | 0.551 | 0.249 | 0.399 | 0.035 | 0.182 | 0.796 | 0.496 | 5 [symbol] |
| 0.318 | 0.377 | 0.053 | 0.330 | 0.389 | 0.623 | 0.463 | 0.428 | 0.016 | 0.136 | 0.478 | 0.625 | 5 [symbol] |
| 0.000 | 0.807 | 0.001 | 0.872 | 0.053 | 0.768 | 0.075 | 0.312 | 0.366 | 0.621 | 0.272 | 0.629 | 5 [symbol] |
| 0.021 | 0.705 | 0.026 | 0.706 | 0.196 | 0.623 | 0.075 | 0.302 | 0.920 | 0.545 | 0.699 | 0.462 | 5 [symbol] |
| 0.005 | 0.766 | 0.011 | 0.753 | 0.085 | 0.768 | 0.600 | 0.428 | 0.615 | 0.583 | 0.606 | 0.462 | 5 [symbol] |
| 0.692 | 0.480 | 0.038 | 0.291 | 0.024 | 0.804 | 0.051 | 0.283 | 0.004 | 0.045 | 0.897 | 0.492 | 5 [symbol] |
| 0.129 | 0.623 | 0.387 | 0.587 | 0.085 | 0.732 | 0.325 | 0.399 | 0.159 | 0.318 | 0.561 | 0.563 | [symbol] |
| 0.556 | 0.439 | 0.049 | 0.314 | 0.085 | 0.732 | 0.173 | 0.341 | 0.027 | 0.182 | 0.699 | 0.425 | [symbol] |
| 0.042 | 0.684 | 0.031 | 0.729 | 0.389 | 0.587 | 0.850 | 0.457 | 0.315 | 0.629 | 0.519 | 0.400 | [symbol] |
| 0.014 | 0.295 | 0.028 | 0.263 | 0.106 | 0.301 | 0.137 | 0.621 | 0.688 | 0.447 | 0.439 | 0.429 | [symbol] |
| 0.004 | 0.234 | 0.005 | 0.211 | 0.095 | 0.301 | 0.011 | 0.795 | 1.000 | 0.492 | 0.272 | 0.363 | [symbol] |
| 0.001 | 0.214 | 0.002 | 0.196 | 0.076 | 0.268 | 0.116 | 0.650 | 0.269 | 0.364 | 0.747 | 0.429 | [symbol] |
| 0.010 | 0.275 | 0.011 | 0.192 | 0.178 | 0.337 | 0.116 | 0.688 | 0.269 | 0.356 | 0.699 | 0.463 | [symbol] |
| 0.042 | 0.316 | 0.034 | 0.287 | 0.360 | 0.373 | 0.346 | 0.601 | 0.482 | 0.402 | 0.478 | 0.463 | [symbol] |
| 0.078 | 0.664 | 0.045 | 0.706 | 0.590 | 0.587 | 0.325 | 0.399 | 0.070 | 0.758 | 0.747 | 0.533 | [symbol] |
| 0.065 | 0.684 | 0.009 | 0.777 | 0.872 | 0.514 | 0.216 | 0.370 | 0.007 | 0.939 | 0.796 | 0.462 | [symbol] |
| 0.005 | 0.746 | 0.000 | 0.872 | 0.872 | 0.518 | 0.438 | 0.447 | 0.027 | 0.803 | 0.175 | 0.296 | [symbol] |
| 0.194 | 0.623 | 0.041 | 0.709 | 0.667 | 0.482 | 0.160 | 0.370 | 0.044 | 0.765 | 0.897 | 0.462 | [symbol] |
| 0.087 | 0.643 | 0.045 | 0.729 | 0.667 | 0.551 | 0.042 | 0.225 | 0.228 | 0.712 | 0.651 | 0.596 | [symbol] |
| 0.061 | 0.664 | 0.016 | 0.753 | 0.872 | 0.478 | 0.107 | 0.312 | 0.044 | 0.811 | 0.747 | 0.525 | 1 [symbol] |
| 0.015 | 0.746 | 0.001 | 0.824 | 0.957 | 0.482 | 0.304 | 0.399 | 0.009 | 0.856 | 0.796 | 0.558 | 1 [symbol] |
| 0.036 | 0.705 | 0.018 | 0.777 | 0.518 | 0.587 | 0.543 | 0.428 | 0.027 | 0.848 | 1.000 | 0.496 | 1 [symbol] |
| 0.005 | 0.725 | 0.004 | 0.824 | 0.178 | 0.696 | 0.137 | 0.341 | 0.228 | 0.674 | 0.747 | 0.462 | 1 [symbol] |
| 0.002 | 0.766 | 0.005 | 0.800 | 0.060 | 0.732 | 0.068 | 0.312 | 0.688 | 0.538 | 0.606 | 0.429 | 1 [symbol] |
| 0.122 | 0.357 | 0.087 | 0.306 | 0.590 | 0.406 | 0.983 | 0.514 | 0.108 | 0.273 | 0.138 | 0.692 | 1 [symbol] |
| 0.028 | 0.295 | 0.011 | 0.211 | 0.554 | 0.446 | 0.173 | 0.630 | 0.088 | 0.265 | 0.220 | 0.367 | 1 [symbol] |
| 0.007 | 0.725 | 0.038 | 0.753 | 0.024 | 0.804 | 0.173 | 0.341 | 0.366 | 0.402 | 0.796 | 0.525 | 1 [symbol] |
| 0.061 | 0.664 | 0.136 | 0.658 | 0.132 | 0.732 | 0.082 | 0.312 | 0.546 | 0.402 | 0.519 | 0.558 | 2 [symbol] |
| 0.001 | 0.807 | 0.001 | 0.872 | 0.067 | 0.696 | 0.062 | 0.283 | 0.421 | 0.583 | 0.606 | 0.429 | 2 [symbol] |
| 0.065 | 0.664 | 0.118 | 0.682 | 0.178 | 0.659 | 0.116 | 0.312 | 0.920 | 0.492 | 0.093 | 0.692 | 3 [symbol] |
| 0.001 | 0.193 | 0.003 | 0.200 | 0.027 | 0.199 | 0.438 | 0.592 | 1.000 | 0.538 | 0.561 | 0.467 | 3 [symbol] |
| 0.015 | 0.275 | 0.019 | 0.291 | 0.178 | 0.301 | 0.390 | 0.621 | 0.366 | 0.364 | 0.796 | 0.496 | 4 [symbol] |
| 0.004 | 0.766 | 0.041 | 0.709 | 0.006 | 0.841 | 0.051 | 0.254 | 0.108 | 0.273 | 0.651 | 0.429 | 53 [symbol] |
| 0.003 | 0.254 | 0.013 | 0.247 | 0.031 | 0.236 | 0.917 | 0.476 | 0.269 | 0.667 | 0.519 | 0.462 | 60 [symbol] |
| 0.000 | 0.152 | 0.001 | 0.196 | 0.001 | 0.058 | 0.126 | 0.679 | 0.108 | 0.712 | 1.000 | 0.529 | 62 [symbol] |
| 0.034 | 0.316 | 0.136 | 0.338 | 0.046 | 0.236 | 0.126 | 0.679 | 0.421 | 0.621 | 0.846 | 0.496 | 66 [symbol] |
| 0.024 | 0.275 | 0.038 | 0.287 | 0.162 | 0.308 | 0.090 | 0.708 | 0.763 | 0.530 | 0.561 | 0.429 | 67 [symbol] |
| 0.001 | 0.214 | 0.005 | 0.223 | 0.021 | 0.199 | 0.390 | 0.592 | 0.841 | 0.447 | 0.138 | 0.300 | 79 [symbol] |
| 0.017 | 0.254 | 0.038 | 0.267 | 0.095 | 0.304 | 0.249 | 0.650 | 1.000 | 0.538 | 0.220 | 0.367 | 88 [symbol] |

[symbol]

indicates data missing or illegible when filed

TABLE B
Biomarkers identified in F1CSH

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | M/ [symbol] |
| P | | P | | P | | P | | P | | P | | (avg [symbol] |
| value | ROC | value | ROC | value | ROC | value | ROC | value | ROC | value | ROC | (Da [symbol] |
| 0.069 | 0.316 | 0.101 | 0.310 | 0.236 | 0.341 | 0.016 | 0.766 | 0.841 | 0.492 | 0.156 | 0.333 | 10 [symbol] |
| 0.028 | 0.705 | 0.080 | 0.682 | 0.076 | 0.732 | 0.346 | 0.399 | 0.688 | 0.447 | 0.302 | 0.625 | 10 [symbol] |
| 0.018 | 0.725 | 0.191 | 0.634 | 0.006 | 0.877 | 0.116 | 0.341 | 0.035 | 0.182 | 0.107 | 0.688 | 11 [symbol] |
| 0.011 | 0.725 | 0.053 | 0.706 | 0.027 | 0.804 | 0.068 | 0.312 | 0.228 | 0.318 | 0.107 | 0.692 | 11 [symbol] |
| 0.004 | 0.766 | 0.018 | 0.753 | 0.024 | 0.804 | 0.438 | 0.428 | 0.421 | 0.364 | 0.033 | 0.788 | 11 [symbol] |
| 0.030 | 0.684 | 0.179 | 0.658 | 0.021 | 0.804 | 0.917 | 0.514 | 0.070 | 0.273 | 0.366 | 0.629 | 12 [symbol] |
| 0.092 | 0.602 | 0.367 | 0.587 | 0.046 | 0.768 | 0.722 | 0.543 | 0.132 | 0.318 | 0.606 | 0.529 | 12 [symbol] |
| 0.042 | 0.684 | 0.348 | 0.587 | 0.008 | 0.841 | 0.543 | 0.601 | 0.003 | 0.045 | 0.897 | 0.496 | 12 [symbol] |
| 0.021 | 0.725 | 0.146 | 0.634 | 0.015 | 0.804 | 0.722 | 0.543 | 0.070 | 0.273 | 0.439 | 0.562 | 12 [symbol] |
| 0.006 | 0.725 | 0.016 | 0.729 | 0.060 | 0.732 | 0.660 | 0.428 | 0.763 | 0.538 | 1.000 | 0.500 | 12 [symbol] |
| 0.002 | 0.766 | 0.004 | 0.800 | 0.060 | 0.768 | 0.572 | 0.399 | 0.841 | 0.439 | 0.949 | 0.467 | 12 [symbol] |
| 0.018 | 0.705 | 0.016 | 0.729 | 0.258 | 0.659 | 0.691 | 0.428 | 0.615 | 0.583 | 0.846 | 0.467 | 12 [symbol] |
| 0.010 | 0.725 | 0.013 | 0.729 | 0.146 | 0.696 | 0.630 | 0.428 | 0.421 | 0.583 | 0.796 | 0.496 | 12 [symbol] |
| 0.003 | 0.766 | 0.014 | 0.753 | 0.027 | 0.732 | 0.630 | 0.457 | 0.421 | 0.402 | 0.699 | 0.496 | 12 [symbol] |
| 0.001 | 0.807 | 0.006 | 0.800 | 0.013 | 0.804 | 0.630 | 0.428 | 0.421 | 0.447 | 0.699 | 0.462 | 12 [symbol] |
| 0.002 | 0.786 | 0.007 | 0.777 | 0.027 | 0.804 | 0.463 | 0.399 | 0.763 | 0.485 | 0.747 | 0.496 | 12 [symbol] |
| 0.002 | 0.766 | 0.012 | 0.777 | 0.018 | 0.804 | 0.660 | 0.476 | 0.688 | 0.447 | 0.272 | 0.363 | 12 [symbol] |
| 0.000 | 0.848 | 0.002 | 0.800 | 0.008 | 0.841 | 0.368 | 0.399 | 0.421 | 0.356 | 0.561 | 0.429 | 12 [symbol] |
| 0.000 | 0.868 | 0.001 | 0.848 | 0.006 | 0.877 | 0.463 | 0.428 | 0.841 | 0.455 | 0.796 | 0.462 | 1 [symbol] |
| 0.000 | 0.827 | 0.002 | 0.824 | 0.006 | 0.841 | 0.572 | 0.428 | 0.615 | 0.402 | 0.949 | 0.525 | 1 [symbol] |
| 0.001 | 0.786 | 0.005 | 0.800 | 0.021 | 0.841 | 0.173 | 0.312 | 0.763 | 0.538 | 0.949 | 0.492 | 1 [symbol] |
| 0.017 | 0.725 | 0.045 | 0.682 | 0.076 | 0.732 | 0.116 | 0.312 | 0.920 | 0.455 | 0.175 | 0.692 | 1 [symbol] |
| 0.004 | 0.234 | 0.001 | 0.152 | 0.419 | 0.409 | 0.160 | 0.650 | 0.035 | 0.136 | 0.245 | 0.658 | 1 [symbol] |
| 0.008 | 0.254 | 0.002 | 0.172 | 0.451 | 0.442 | 0.148 | 0.679 | 0.016 | 0.136 | 0.651 | 0.558 | 1 [symbol] |
| 0.047 | 0.275 | 0.013 | 0.239 | 0.788 | 0.442 | 0.160 | 0.630 | 0.027 | 0.182 | 0.439 | 0.429 | 1 [symbol] |
| 0.012 | 0.746 | 0.034 | 0.753 | 0.067 | 0.732 | 0.201 | 0.370 | 0.482 | 0.402 | 0.478 | 0.592 | 1 [symbol] |
| 0.042 | 0.705 | 0.179 | 0.658 | 0.041 | 0.768 | 0.304 | 0.370 | 0.159 | 0.311 | 0.519 | 0.596 | 1 [symbol] |
| 0.004 | 0.214 | 0.031 | 0.263 | 0.013 | 0.159 | 0.660 | 0.457 | 0.269 | 0.667 | 0.699 | 0.463 | 1 [symbol] |
| 0.000 | 0.827 | 0.002 | 0.848 | 0.015 | 0.804 | 0.082 | 0.341 | 0.546 | 0.364 | 0.053 | 0.758 | 1 [symbol] |
| 0.092 | 0.643 | 0.246 | 0.587 | 0.106 | 0.696 | 0.516 | 0.601 | 0.191 | 0.273 | 0.561 | 0.562 | 20 [symbol] |
| 0.022 | 0.705 | 0.045 | 0.729 | 0.118 | 0.732 | 0.160 | 0.341 | 0.482 | 0.402 | 0.156 | 0.692 | 25 [symbol] |
| 0.012 | 0.725 | 0.041 | 0.729 | 0.053 | 0.768 | 0.325 | 0.399 | 0.546 | 0.447 | 0.093 | 0.696 | 25 [symbol] |
| 0.001 | 0.827 | 0.003 | 0.800 | 0.027 | 0.804 | 0.046 | 0.283 | 0.615 | 0.447 | 0.138 | 0.692 | 26 [symbol] |
| 0.044 | 0.684 | 0.058 | 0.682 | 0.236 | 0.659 | 0.785 | 0.486 | 0.421 | 0.621 | 0.175 | 0.329 | 27 [symbol] |
| 0.003 | 0.786 | 0.001 | 0.848 | 0.196 | 0.659 | 0.390 | 0.428 | 0.366 | 0.621 | 0.121 | 0.300 | 28 [symbol] |
| 0.010 | 0.746 | 0.003 | 0.800 | 0.419 | 0.587 | 0.850 | 0.514 | 0.421 | 0.667 | 0.197 | 0.329 | 29 [symbol] |
| 0.030 | 0.705 | 0.011 | 0.753 | 0.590 | 0.627 | 0.051 | 0.283 | 0.027 | 0.848 | 0.302 | 0.658 | 30 [symbol] |
| 0.024 | 0.725 | 0.021 | 0.753 | 0.282 | 0.663 | 0.068 | 0.312 | 0.920 | 0.492 | 0.519 | 0.592 | 31 [symbol] |
| 0.001 | 0.214 | 0.001 | 0.176 | 0.046 | 0.236 | 0.148 | 0.650 | 0.546 | 0.447 | 0.333 | 0.367 | 39 [symbol] |
| 0.042 | 0.316 | 0.019 | 0.267 | 0.554 | 0.409 | 0.325 | 0.601 | 0.044 | 0.227 | 0.897 | 0.492 | 44 [symbol] |
| 0.039 | 0.316 | 0.018 | 0.267 | 0.554 | 0.409 | 0.249 | 0.630 | 0.044 | 0.227 | 0.897 | 0.492 | 44 [symbol] |
| 0.019 | 0.705 | 0.118 | 0.658 | 0.021 | 0.768 | 0.068 | 0.312 | 0.191 | 0.318 | 0.220 | 0.625 | 50 [symbol] |
| 0.000 | 0.889 | 0.000 | 0.872 | 0.003 | 0.877 | 0.011 | 0.225 | 0.269 | 0.356 | 0.846 | 0.525 | 51 [symbol] |
| 0.007 | 0.725 | 0.087 | 0.658 | 0.005 | 0.877 | 0.883 | 0.534 | 0.191 | 0.273 | 0.220 | 0.329 | 53 [symbol] |
| 0.008 | 0.275 | 0.018 | 0.243 | 0.076 | 0.272 | 0.630 | 0.534 | 0.688 | 0.538 | 0.272 | 0.358 | 58 [symbol] |
| 0.001 | 0.193 | 0.003 | 0.196 | 0.018 | 0.199 | 0.463 | 0.592 | 0.546 | 0.583 | 0.699 | 0.429 | 59 [symbol] |
| 0.012 | 0.295 | 0.041 | 0.291 | 0.053 | 0.236 | 0.983 | 0.534 | 0.546 | 0.583 | 1.000 | 0.529 | 60 [symbol] |
| 0.004 | 0.254 | 0.031 | 0.287 | 0.010 | 0.127 | 0.346 | 0.611 | 0.421 | 0.629 | 0.747 | 0.567 | 62 [symbol] |
| 0.010 | 0.254 | 0.013 | 0.263 | 0.146 | 0.344 | 0.022 | 0.766 | 0.763 | 0.538 | 0.478 | 0.458 | 67 [symbol] |
| 0.004 | 0.234 | 0.006 | 0.196 | 0.085 | 0.236 | 0.042 | 0.708 | 0.615 | 0.409 | 0.478 | 0.396 | 72 [symbol] |
| 0.018 | 0.295 | 0.014 | 0.243 | 0.282 | 0.337 | 0.046 | 0.717 | 0.421 | 0.356 | 0.699 | 0.458 | 74 [symbol] |
| 0.001 | 0.193 | 0.002 | 0.176 | 0.046 | 0.272 | 0.187 | 0.650 | 0.546 | 0.402 | 0.156 | 0.300 | 78 [symbol] |
| 0.009 | 0.254 | 0.016 | 0.243 | 0.106 | 0.268 | 0.075 | 0.737 | 0.688 | 0.409 | 0.272 | 0.400 | 88 [symbol] |
| 0.013 | 0.705 | 0.038 | 0.706 | 0.067 | 0.732 | 0.090 | 0.312 | 0.841 | 0.492 | 0.272 | 0.629 | 96 [symbol] |

[symbol]

indicates data missing or illegible when filed

TABLE C
Biomarkers identified in F1ISL

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | M/ [symbol] |
| P | | P | | P | | P | | P | | P | | (av [symbol] |
| value | ROC | value | ROC | value | ROC | value | ROC | value | ROC | value | ROC | (D [symbol] |
| 0.121 | 0.322 | 0.061 | 0.739 | 0.831 | 0.466 | 0.815 | 0.432 | 0.062 | 0.125 | 0.453 | 0.429 | [symbol] |
| 0.005 | 0.777 | 0.055 | 0.261 | 0.009 | 0.920 | 0.482 | 0.432 | 0.126 | 0.250 | 0.375 | 0.598 | [symbol] |
| 0.061 | 0.307 | 0.010 | 0.773 | 0.831 | 0.523 | 0.223 | 0.670 | 0.042 | 0.125 | 0.101 | 0.313 | [symbol] |
| 0.000 | 0.102 | 0.001 | 0.875 | 0.013 | 0.125 | 0.325 | 0.636 | 0.396 | 0.313 | 0.682 | 0.429 | [symbol] |
| 0.639 | 0.527 | 0.778 | 0.534 | 0.177 | 0.693 | 0.348 | 0.398 | 0.126 | 0.250 | 0.065 | 0.705 | [symbol] |
| 0.280 | 0.375 | 0.024 | 0.773 | 0.201 | 0.636 | 0.425 | 0.602 | 0.062 | 0.188 | 0.339 | 0.670 | [symbol] |
| 0.387 | 0.598 | 0.639 | 0.568 | 0.016 | 0.864 | 0.373 | 0.398 | 0.042 | 0.125 | 0.539 | 0.589 | [symbol] |
| 0.044 | 0.307 | 0.019 | 0.739 | 0.670 | 0.409 | 0.815 | 0.466 | 0.308 | 0.313 | 0.495 | 0.393 | [symbol] |
| 0.016 | 0.754 | 0.101 | 0.330 | 0.023 | 0.807 | 0.925 | 0.466 | 0.126 | 0.250 | 0.056 | 0.732 | [symbol] |
| 0.037 | 0.303 | 0.015 | 0.807 | 0.670 | 0.409 | 0.241 | 0.636 | 0.234 | 0.313 | 0.375 | 0.598 | [symbol] |
| 0.105 | 0.371 | 0.028 | 0.773 | 0.887 | 0.466 | 0.302 | 0.636 | 0.089 | 0.188 | 0.413 | 0.563 | [symbol] |
| 0.746 | 0.462 | 0.325 | 0.602 | 0.394 | 0.636 | 0.673 | 0.568 | 0.234 | 0.250 | 1.000 | 0.491 | [symbol] |
| 0.105 | 0.333 | 0.083 | 0.705 | 0.570 | 0.432 | 0.399 | 0.602 | 0.497 | 0.438 | 0.946 | 0.455 | [symbol] |
| 0.000 | 0.083 | 0.000 | 0.875 | 0.065 | 0.239 | 0.022 | 0.773 | 0.089 | 0.188 | 0.946 | 0.527 | 3 [symbol] |
| 0.025 | 0.258 | 0.007 | 0.807 | 0.776 | 0.409 | 0.189 | 0.636 | 0.042 | 0.125 | 0.413 | 0.384 | 3 [symbol] |
| 0.044 | 0.277 | 0.015 | 0.773 | 0.776 | 0.409 | 0.205 | 0.602 | 0.042 | 0.125 | 0.306 | 0.357 | 3 [symbol] |
| 0.220 | 0.367 | 0.039 | 0.773 | 0.477 | 0.636 | 0.542 | 0.602 | 0.042 | 0.125 | 0.133 | 0.321 | 3 [symbol] |
| 0.037 | 0.299 | 0.013 | 0.807 | 0.722 | 0.466 | 0.399 | 0.602 | 0.011 | 0.063 | 0.133 | 0.277 | 4 [symbol] |
| 0.084 | 0.307 | 0.019 | 0.773 | 0.887 | 0.523 | 0.122 | 0.636 | 0.062 | 0.188 | 0.375 | 0.411 | 4 [symbol] |
| 0.037 | 0.280 | 0.017 | 0.807 | 0.619 | 0.409 | 0.189 | 0.670 | 0.308 | 0.375 | 0.838 | 0.429 | 4 [symbol] |
| 0.234 | 0.636 | 0.743 | 0.500 | 0.065 | 0.750 | 0.743 | 0.432 | 0.126 | 0.250 | 0.029 | 0.250 | 4 [symbol] |
| 0.589 | 0.549 | 0.453 | 0.602 | 0.028 | 0.864 | 0.606 | 0.568 | 0.011 | 0.063 | 1.000 | 0.491 | 4 [symbol] |
| 0.028 | 0.731 | 0.015 | 0.193 | 0.522 | 0.636 | 0.373 | 0.398 | 0.308 | 0.625 | 0.101 | 0.696 | 4 [symbol] |
| 0.023 | 0.258 | 0.049 | 0.773 | 0.136 | 0.239 | 0.146 | 0.705 | 0.308 | 0.688 | 0.453 | 0.420 | 4 [symbol] |
| 0.011 | 0.235 | 0.022 | 0.773 | 0.118 | 0.295 | 0.325 | 0.602 | 0.174 | 0.250 | 0.453 | 0.598 | 5 [symbol] |
| 0.149 | 0.663 | 0.189 | 0.330 | 0.394 | 0.636 | 0.606 | 0.432 | 0.610 | 0.625 | 0.219 | 0.696 | 5 [symbol] |
| 0.149 | 0.330 | 0.006 | 0.841 | 0.177 | 0.750 | 0.851 | 0.466 | 0.007 | 0.000 | 0.116 | 0.741 | 5 [symbol] |
| 0.105 | 0.311 | 0.001 | 0.875 | 0.076 | 0.807 | 0.281 | 0.364 | 0.007 | 0.000 | 0.891 | 0.536 | 5 [symbol] |
| 0.090 | 0.311 | 0.002 | 0.841 | 0.201 | 0.750 | 0.743 | 0.432 | 0.007 | 0.000 | 0.633 | 0.563 | 5 [symbol] |
| 0.331 | 0.402 | 0.004 | 0.841 | 0.013 | 0.864 | 0.673 | 0.500 | 0.007 | 0.000 | 0.375 | 0.563 | 5 [symbol] |
| 0.639 | 0.568 | 0.482 | 0.602 | 0.047 | 0.807 | 0.425 | 0.398 | 0.042 | 0.125 | 0.891 | 0.491 | 6 [symbol] |
| 0.007 | 0.220 | 0.002 | 0.841 | 0.522 | 0.409 | 0.017 | 0.773 | 0.062 | 0.125 | 0.838 | 0.491 | 6 [symbol] |
| 0.077 | 0.348 | 0.035 | 0.739 | 0.776 | 0.409 | 0.039 | 0.739 | 0.089 | 0.188 | 0.585 | 0.455 | 6 [symbol] |
| 0.025 | 0.284 | 0.004 | 0.841 | 1.000 | 0.466 | 0.061 | 0.670 | 0.042 | 0.125 | 0.682 | 0.563 | 6 [symbol] |
| 0.006 | 0.174 | 0.002 | 0.841 | 0.434 | 0.409 | 0.055 | 0.739 | 0.089 | 0.188 | 0.375 | 0.554 | 6 [symbol] |
| 0.019 | 0.261 | 0.007 | 0.807 | 0.619 | 0.466 | 0.122 | 0.705 | 0.174 | 0.250 | 0.306 | 0.625 | 6 [symbol] |
| 0.012 | 0.261 | 0.003 | 0.841 | 0.670 | 0.409 | 0.049 | 0.739 | 0.042 | 0.125 | 0.339 | 0.625 | 6 [symbol] |
| 0.028 | 0.705 | 0.024 | 0.227 | 0.356 | 0.636 | 0.743 | 0.568 | 0.497 | 0.625 | 0.413 | 0.393 | 7 [symbol] |
| 0.056 | 0.708 | 0.024 | 0.227 | 0.722 | 0.523 | 0.888 | 0.534 | 0.396 | 0.625 | 0.495 | 0.393 | 7 [symbol] |
| 0.195 | 0.640 | 0.031 | 0.193 | 0.477 | 0.375 | 0.223 | 0.364 | 0.017 | 0.938 | 0.838 | 0.491 | 9 [symbol] |
| 0.007 | 0.795 | 0.001 | 0.125 | 0.722 | 0.523 | 0.055 | 0.295 | 0.174 | 0.750 | 0.733 | 0.464 | 9 [symbol] |
| 0.023 | 0.727 | 0.009 | 0.227 | 0.619 | 0.636 | 0.055 | 0.261 | 0.062 | 0.813 | 0.495 | 0.607 | 9 [symbol] |
| 0.009 | 0.773 | 0.002 | 0.159 | 0.570 | 0.636 | 0.002 | 0.125 | 0.089 | 0.813 | 0.339 | 0.607 | 9 [symbol] |
| 0.002 | 0.795 | 0.002 | 0.125 | 0.155 | 0.693 | 0.002 | 0.125 | 0.734 | 0.563 | 0.152 | 0.670 | 9 [symbol] |
| 0.047 | 0.686 | 0.015 | 0.193 | 0.831 | 0.523 | 0.241 | 0.364 | 0.308 | 0.688 | 0.076 | 0.741 | 10 [symbol] |
| 0.007 | 0.239 | 0.003 | 0.841 | 0.434 | 0.409 | 0.005 | 0.841 | 0.062 | 0.188 | 0.682 | 0.571 | 13 [symbol] |
| 0.000 | 0.083 | 0.000 | 0.909 | 0.007 | 0.091 | 0.002 | 0.875 | 0.734 | 0.438 | 0.539 | 0.420 | 14 [symbol] |
| 0.007 | 0.239 | 0.035 | 0.773 | 0.033 | 0.148 | 0.425 | 0.398 | 0.396 | 0.625 | 0.891 | 0.464 | 15 [symbol] |
| 0.097 | 0.352 | 0.067 | 0.739 | 0.619 | 0.432 | 0.888 | 0.500 | 0.396 | 0.375 | 0.009 | 0.839 | 23 [symbol] |
| 0.033 | 0.307 | 0.031 | 0.773 | 0.356 | 0.375 | 0.815 | 0.500 | 0.610 | 0.375 | 0.029 | 0.777 | 23 [symbol] |
| 0.220 | 0.394 | 0.011 | 0.807 | 0.155 | 0.750 | 0.281 | 0.636 | 0.017 | 0.063 | 1.000 | 0.491 | 27 [symbol] |
| 0.207 | 0.398 | 0.174 | 0.670 | 0.670 | 0.432 | 0.815 | 0.568 | 0.610 | 0.375 | 0.682 | 0.455 | 34 [symbol] |
| 0.012 | 0.239 | 0.004 | 0.841 | 0.522 | 0.375 | 0.925 | 0.500 | 0.174 | 0.250 | 0.633 | 0.429 | 3 [symbol] |
| 0.006 | 0.197 | 0.009 | 0.807 | 0.136 | 0.261 | 0.281 | 0.602 | 1.000 | 0.500 | 0.838 | 0.527 | 4 [symbol] |
| 0.004 | 0.189 | 0.011 | 0.807 | 0.055 | 0.239 | 0.673 | 0.500 | 0.396 | 0.375 | 0.453 | 0.607 | 4 [symbol] |
| 0.009 | 0.235 | 0.007 | 0.807 | 0.320 | 0.352 | 0.067 | 0.705 | 0.174 | 0.250 | 1.000 | 0.491 | 4 [symbol] |
| 0.013 | 0.258 | 0.015 | 0.773 | 0.227 | 0.295 | 0.189 | 0.670 | 0.174 | 0.250 | 0.339 | 0.384 | 4 [symbol] |
| 0.009 | 0.212 | 0.013 | 0.807 | 0.177 | 0.295 | 0.189 | 0.670 | 0.174 | 0.250 | 0.946 | 0.527 | 4 [symbol] |
| 0.007 | 0.216 | 0.013 | 0.773 | 0.118 | 0.239 | 0.022 | 0.773 | 0.174 | 0.250 | 0.891 | 0.527 | 4 [symbol] |
| 0.003 | 0.216 | 0.005 | 0.807 | 0.102 | 0.239 | 0.101 | 0.670 | 0.497 | 0.375 | 0.585 | 0.563 | 4 [symbol] |
| 0.044 | 0.284 | 0.022 | 0.739 | 0.619 | 0.432 | 0.075 | 0.705 | 0.308 | 0.313 | 0.495 | 0.634 | 5 [symbol] |
| 0.019 | 0.303 | 0.004 | 0.875 | 0.776 | 0.432 | 0.189 | 0.636 | 0.234 | 0.250 | 0.946 | 0.500 | 7 [symbol] |
| 0.001 | 0.125 | 0.003 | 0.875 | 0.023 | 0.182 | 0.111 | 0.705 | 0.610 | 0.625 | 0.838 | 0.464 | 7 [symbol] |

[symbol]

indicates data missing or illegible when filed

TABLE D
Biomarkers identified in F1ISH

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | M/Z |
| P | | P | | P | | P | | P | | P | | ( [symbol] |
| value | ROC | value | ROC | value | ROC | value | ROC | value | ROC | value | ROC | (D [symbol] |
| 0.017 | 0.231 | 0.013 | 0.193 | 0.289 | 0.318 | 0.093 | 0.760 | 0.558 | 0.413 | 0.891 | 0.464 | 1 [symbol] |
| 0.082 | 0.304 | 0.061 | 0.261 | 0.492 | 0.409 | 0.059 | 0.760 | 0.661 | 0.413 | 0.891 | 0.464 | 1 [symbol] |
| 0.010 | 0.255 | 0.002 | 0.159 | 0.574 | 0.423 | 0.083 | 0.695 | 0.143 | 0.263 | 0.048 | 0.741 | 1 [symbol] |
| 0.013 | 0.255 | 0.003 | 0.159 | 0.533 | 0.377 | 0.059 | 0.727 | 0.143 | 0.263 | 0.056 | 0.741 | 1 [symbol] |
| 0.007 | 0.231 | 0.002 | 0.125 | 0.417 | 0.377 | 0.083 | 0.695 | 0.306 | 0.313 | 0.017 | 0.768 | 1 [symbol] |
| 0.070 | 0.304 | 0.019 | 0.227 | 0.851 | 0.455 | 0.508 | 0.571 | 0.380 | 0.363 | 0.020 | 0.804 | 1 [symbol] |
| 0.290 | 0.378 | 0.111 | 0.295 | 0.851 | 0.500 | 0.959 | 0.506 | 0.306 | 0.313 | 0.172 | 0.634 | 1 [symbol] |
| 0.172 | 0.671 | 0.673 | 0.534 | 0.053 | 0.773 | 0.114 | 0.344 | 0.107 | 0.213 | 0.041 | 0.732 | 1 [symbol] |
| 0.207 | 0.647 | 0.888 | 0.534 | 0.034 | 0.773 | 0.415 | 0.442 | 0.079 | 0.213 | 0.453 | 0.598 | 1 [symbol] |
| 0.116 | 0.647 | 0.511 | 0.602 | 0.046 | 0.773 | 0.878 | 0.506 | 0.188 | 0.313 | 0.088 | 0.705 | 1 [symbol] |
| 0.162 | 0.622 | 0.743 | 0.534 | 0.034 | 0.818 | 0.359 | 0.344 | 0.143 | 0.213 | 0.453 | 0.634 | 1 [symbol] |
| 0.539 | 0.427 | 0.083 | 0.295 | 0.236 | 0.682 | 0.919 | 0.503 | 0.013 | 0.113 | 0.585 | 0.554 | 1 [symbol] |
| 0.290 | 0.378 | 0.031 | 0.227 | 0.349 | 0.636 | 0.610 | 0.568 | 0.005 | 0.013 | 0.453 | 0.393 | 1 [symbol] |
| 0.101 | 0.304 | 0.024 | 0.227 | 1.000 | 0.500 | 0.476 | 0.601 | 0.143 | 0.263 | 0.116 | 0.286 | 1 [symbol] |
| 0.031 | 0.745 | 0.055 | 0.739 | 0.170 | 0.682 | 0.285 | 0.633 | 1.000 | 0.450 | 0.172 | 0.321 | 1 [symbol] |
| 0.034 | 0.280 | 0.007 | 0.159 | 0.803 | 0.455 | 0.093 | 0.731 | 0.028 | 0.113 | 0.195 | 0.321 | 1 [symbol] |
| 0.065 | 0.304 | 0.019 | 0.193 | 0.803 | 0.468 | 0.185 | 0.695 | 0.107 | 0.213 | 0.195 | 0.321 | 1 [symbol] |
| 0.006 | 0.231 | 0.002 | 0.159 | 0.349 | 0.364 | 0.006 | 0.825 | 0.028 | 0.113 | 0.133 | 0.321 | 1 [symbol] |
| 0.003 | 0.182 | 0.003 | 0.159 | 0.170 | 0.286 | 0.008 | 0.857 | 0.380 | 0.313 | 0.088 | 0.321 | 1 [symbol] |
| 0.012 | 0.231 | 0.028 | 0.227 | 0.092 | 0.273 | 0.074 | 0.731 | 0.661 | 0.413 | 0.024 | 0.214 | 1 [symbol] |
| 0.024 | 0.255 | 0.083 | 0.295 | 0.070 | 0.286 | 0.103 | 0.727 | 0.306 | 0.700 | 0.008 | 0.179 | 1 [symbol] |
| 0.034 | 0.329 | 0.055 | 0.295 | 0.190 | 0.332 | 0.103 | 0.698 | 1.000 | 0.500 | 0.029 | 0.214 | 1 [symbol] |
| 0.037 | 0.304 | 0.083 | 0.295 | 0.134 | 0.286 | 0.185 | 0.698 | 0.770 | 0.550 | 0.029 | 0.250 | 1 [symbol] |
| 0.005 | 0.231 | 0.024 | 0.227 | 0.034 | 0.195 | 0.285 | 0.633 | 0.380 | 0.650 | 0.152 | 0.321 | 1 [symbol] |
| 0.006 | 0.231 | 0.022 | 0.227 | 0.046 | 0.195 | 0.203 | 0.633 | 0.242 | 0.700 | 0.172 | 0.321 | 1 [symbol] |
| 0.322 | 0.598 | 0.925 | 0.466 | 0.053 | 0.818 | 0.386 | 0.601 | 0.028 | 0.163 | 0.838 | 0.563 | [symbol] |
| 0.322 | 0.378 | 0.035 | 0.227 | 0.318 | 0.682 | 0.721 | 0.536 | 0.079 | 0.213 | 0.539 | 0.571 | [symbol] |
| 0.088 | 0.280 | 0.024 | 0.227 | 0.901 | 0.500 | 0.575 | 0.568 | 0.057 | 0.213 | 0.785 | 0.571 | [symbol] |
| 0.082 | 0.671 | 0.122 | 0.636 | 0.261 | 0.682 | 0.221 | 0.344 | 0.884 | 0.500 | 0.246 | 0.634 | [symbol] |
| 0.562 | 0.549 | 0.963 | 0.500 | 0.261 | 0.636 | 0.919 | 0.503 | 0.188 | 0.263 | 0.453 | 0.393 | [symbol] |
| 0.000 | 0.157 | 0.004 | 0.125 | 0.005 | 0.105 | 0.022 | 0.760 | 0.770 | 0.450 | 0.633 | 0.563 | [symbol] |
| 0.000 | 0.133 | 0.001 | 0.125 | 0.013 | 0.150 | 0.333 | 0.568 | 0.770 | 0.462 | 0.339 | 0.321 | [symbol] |
| 0.000 | 0.108 | 0.001 | 0.125 | 0.021 | 0.195 | 0.032 | 0.760 | 0.306 | 0.363 | 0.585 | 0.393 | [symbol] |
| 0.017 | 0.231 | 0.031 | 0.227 | 0.134 | 0.318 | 0.093 | 0.698 | 0.380 | 0.313 | 0.891 | 0.491 | [symbol] |
| 0.009 | 0.231 | 0.017 | 0.193 | 0.119 | 0.273 | 0.262 | 0.633 | 0.306 | 0.313 | 0.585 | 0.420 | [symbol] |
| 0.010 | 0.206 | 0.028 | 0.227 | 0.081 | 0.227 | 0.154 | 0.666 | 0.661 | 0.363 | 0.682 | 0.455 | [symbol] |
| 0.029 | 0.280 | 0.044 | 0.261 | 0.190 | 0.332 | 0.541 | 0.601 | 0.464 | 0.350 | 0.785 | 0.455 | [symbol] |
| 0.065 | 0.304 | 0.019 | 0.227 | 0.803 | 0.455 | 0.017 | 0.792 | 0.040 | 0.163 | 0.785 | 0.455 | [symbol] |
| 0.000 | 0.133 | 0.000 | 0.057 | 0.070 | 0.255 | 0.014 | 0.792 | 0.558 | 0.413 | 0.495 | 0.420 | [symbol] |
| 0.000 | 0.084 | 0.000 | 0.023 | 0.039 | 0.209 | 0.083 | 0.731 | 0.770 | 0.450 | 0.375 | 0.357 | [symbol] |
| 0.000 | 0.084 | 0.000 | 0.057 | 0.010 | 0.164 | 0.017 | 0.792 | 0.661 | 0.450 | 0.838 | 0.491 | [symbol] |
| 0.000 | 0.084 | 0.000 | 0.057 | 0.009 | 0.164 | 0.139 | 0.666 | 1.000 | 0.500 | 0.539 | 0.420 | [symbol] |
| 0.000 | 0.059 | 0.000 | 0.057 | 0.004 | 0.118 | 0.067 | 0.695 | 0.380 | 0.650 | 0.246 | 0.348 | [symbol] |
| 0.000 | 0.157 | 0.004 | 0.159 | 0.004 | 0.073 | 0.017 | 0.792 | 0.306 | 0.650 | 0.682 | 0.491 | [symbol] |
| 0.000 | 0.108 | 0.002 | 0.125 | 0.002 | 0.073 | 0.610 | 0.536 | 0.143 | 0.750 | 0.633 | 0.455 | [symbol] |
| 0.015 | 0.280 | 0.011 | 0.193 | 0.289 | 0.332 | 0.919 | 0.536 | 0.380 | 0.350 | 0.682 | 0.491 | 6 [symbol] |
| 0.000 | 0.157 | 0.002 | 0.125 | 0.021 | 0.150 | 0.032 | 0.760 | 0.770 | 0.550 | 0.682 | 0.527 | 6 [symbol] |
| 0.004 | 0.206 | 0.003 | 0.125 | 0.190 | 0.286 | 0.059 | 0.731 | 0.558 | 0.400 | 0.633 | 0.393 | 6 [symbol] |
| 0.012 | 0.206 | 0.015 | 0.193 | 0.170 | 0.318 | 0.093 | 0.731 | 0.464 | 0.413 | 0.682 | 0.429 | 7 [symbol] |
| 0.026 | 0.329 | 0.022 | 0.261 | 0.318 | 0.377 | 0.114 | 0.698 | 0.242 | 0.300 | 0.495 | 0.455 | 7 [symbol] |
| 0.001 | 0.182 | 0.004 | 0.159 | 0.025 | 0.195 | 0.093 | 0.727 | 0.380 | 0.363 | 0.088 | 0.286 | 7 [symbol] |
| 0.002 | 0.182 | 0.003 | 0.159 | 0.092 | 0.273 | 0.169 | 0.633 | 0.380 | 0.363 | 0.172 | 0.321 | 8 [symbol] |
| 0.008 | 0.231 | 0.011 | 0.193 | 0.134 | 0.286 | 0.041 | 0.760 | 0.884 | 0.463 | 0.891 | 0.464 | 8 [symbol] |
| 0.026 | 0.255 | 0.061 | 0.261 | 0.119 | 0.273 | 0.067 | 0.698 | 1.000 | 0.462 | 0.453 | 0.393 | 9 [symbol] |
| 0.070 | 0.304 | 0.019 | 0.193 | 0.851 | 0.468 | 0.221 | 0.633 | 0.242 | 0.300 | 0.891 | 0.464 | 13 [symbol] |

[symbol]

indicates data missing or illegible when filed

TABLE E
Biomarkers identified in F3CSL

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | M/ [symbol] |
| P | | P | | P | | P | | P | | P | | (av [symbol] |
| value | ROC | value | ROC | value | ROC | value | ROC | value | ROC | value | ROC | (D [symbol] |
| 0.024 | 0.316 | 0.013 | 0.243 | 0.419 | 0.373 | 0.722 | 0.563 | 0.228 | 0.318 | 0.161 | 0.344 | 2 [symbol] |
| 0.203 | 0.377 | 0.031 | 0.287 | 0.518 | 0.587 | 0.414 | 0.621 | 0.056 | 0.227 | 0.122 | 0.271 | 2 [symbol] |
| 0.026 | 0.705 | 0.191 | 0.634 | 0.013 | 0.804 | 0.516 | 0.399 | 0.159 | 0.273 | 0.269 | 0.354 | 2 [symbol] |
| 0.098 | 0.664 | 0.045 | 0.729 | 0.747 | 0.554 | 0.082 | 0.312 | 0.228 | 0.712 | 0.768 | 0.438 | [symbol] |
| 0.098 | 0.643 | 0.348 | 0.563 | 0.060 | 0.732 | 0.753 | 0.534 | 0.159 | 0.318 | 0.161 | 0.271 | [symbol] |
| 0.073 | 0.664 | 0.408 | 0.587 | 0.021 | 0.804 | 0.950 | 0.514 | 0.056 | 0.227 | 0.417 | 0.396 | [symbol] |
| 0.122 | 0.643 | 0.387 | 0.611 | 0.076 | 0.768 | 0.232 | 0.370 | 0.228 | 0.356 | 0.417 | 0.396 | [symbol] |
| 0.050 | 0.664 | 0.167 | 0.658 | 0.067 | 0.732 | 0.046 | 0.283 | 0.421 | 0.402 | 0.376 | 0.427 | [symbol] |
| 0.003 | 0.746 | 0.024 | 0.729 | 0.013 | 0.804 | 0.232 | 0.399 | 0.315 | 0.402 | 0.883 | 0.510 | [symbol] |
| 0.213 | 0.398 | 0.619 | 0.429 | 0.085 | 0.232 | 0.098 | 0.679 | 0.269 | 0.674 | 0.768 | 0.479 | [symbol] |
| 0.028 | 0.684 | 0.012 | 0.753 | 0.518 | 0.623 | 0.011 | 0.196 | 0.191 | 0.720 | 0.338 | 0.667 | [symbol] |
| 0.092 | 0.664 | 0.045 | 0.682 | 0.706 | 0.587 | 0.042 | 0.312 | 0.366 | 0.667 | 0.210 | 0.677 | [symbol] |
| 0.047 | 0.643 | 0.008 | 0.777 | 1.000 | 0.514 | 0.090 | 0.283 | 0.044 | 0.803 | 0.825 | 0.427 | [symbol] |
| 0.044 | 0.684 | 0.026 | 0.729 | 0.484 | 0.623 | 0.062 | 0.312 | 0.070 | 0.765 | 0.376 | 0.385 | [symbol] |
| 0.036 | 0.684 | 0.094 | 0.682 | 0.095 | 0.732 | 0.630 | 0.457 | 0.841 | 0.500 | 0.269 | 0.312 | [symbol] |
| 0.028 | 0.275 | 0.074 | 0.310 | 0.085 | 0.308 | 0.368 | 0.592 | 0.615 | 0.576 | 0.338 | 0.604 | [symbol] |
| 0.001 | 0.214 | 0.003 | 0.219 | 0.015 | 0.232 | 0.600 | 0.543 | 0.920 | 0.538 | 0.555 | 0.396 | [symbol] |
| 0.010 | 0.254 | 0.006 | 0.219 | 0.282 | 0.377 | 0.051 | 0.708 | 0.269 | 0.318 | 0.461 | 0.354 | [symbol] |
| 0.001 | 0.193 | 0.001 | 0.152 | 0.106 | 0.304 | 0.630 | 0.437 | 0.132 | 0.227 | 0.768 | 0.510 | [symbol] |
| 0.012 | 0.316 | 0.041 | 0.287 | 0.053 | 0.264 | 0.018 | 0.737 | 0.615 | 0.545 | 0.238 | 0.625 | [symbol] |
| 0.004 | 0.234 | 0.008 | 0.219 | 0.076 | 0.264 | 0.010 | 0.795 | 0.615 | 0.447 | 0.269 | 0.635 | [symbol] |
| 0.006 | 0.254 | 0.016 | 0.239 | 0.060 | 0.264 | 0.014 | 0.766 | 0.688 | 0.492 | 0.461 | 0.583 | [symbol] |
| 0.082 | 0.336 | 0.045 | 0.291 | 0.628 | 0.442 | 0.390 | 0.592 | 0.269 | 0.318 | 0.027 | 0.833 | [symbol] |
| 0.022 | 0.295 | 0.053 | 0.287 | 0.095 | 0.268 | 0.785 | 0.514 | 0.920 | 0.492 | 0.658 | 0.563 | [symbol] |
| 0.011 | 0.275 | 0.041 | 0.287 | 0.046 | 0.225 | 0.051 | 0.737 | 0.688 | 0.545 | 0.461 | 0.635 | 1 [symbol] |
| 0.004 | 0.214 | 0.004 | 0.215 | 0.118 | 0.264 | 0.022 | 0.756 | 0.088 | 0.273 | 0.122 | 0.750 | 1 [symbol] |
| 0.042 | 0.357 | 0.094 | 0.310 | 0.118 | 0.301 | 0.187 | 0.679 | 1.000 | 0.492 | 0.055 | 0.792 | 1 [symbol] |
| 0.008 | 0.254 | 0.028 | 0.263 | 0.041 | 0.228 | 0.137 | 0.679 | 0.841 | 0.500 | 0.376 | 0.635 | 28 [symbol] |
| 0.007 | 0.254 | 0.021 | 0.263 | 0.053 | 0.264 | 0.173 | 0.650 | 0.763 | 0.538 | 0.507 | 0.594 | 29 [symbol] |
| 0.436 | 0.439 | 0.191 | 0.354 | 0.706 | 0.558 | 0.062 | 0.698 | 0.615 | 0.439 | 0.105 | 0.271 | 40 [symbol] |
| 0.092 | 0.357 | 0.101 | 0.358 | 0.360 | 0.373 | 0.572 | 0.543 | 0.920 | 0.545 | 0.768 | 0.479 | 41 [symbol] |
| 0.015 | 0.295 | 0.101 | 0.314 | 0.018 | 0.192 | 0.325 | 0.360 | 0.688 | 0.545 | 0.210 | 0.677 | 56 [symbol] |
| 0.022 | 0.275 | 0.074 | 0.314 | 0.060 | 0.232 | 0.818 | 0.534 | 0.841 | 0.538 | 0.768 | 0.521 | 56 [symbol] |
| 0.050 | 0.316 | 0.038 | 0.267 | 0.419 | 0.409 | 0.201 | 0.650 | 0.482 | 0.409 | 0.077 | 0.313 | 60 [symbol] |
| 0.057 | 0.664 | 0.008 | 0.781 | 0.914 | 0.518 | 0.267 | 0.389 | 0.044 | 0.765 | 0.883 | 0.479 | 78 [symbol] |
| 0.019 | 0.705 | 0.016 | 0.753 | 0.282 | 0.627 | 0.160 | 0.341 | 0.615 | 0.583 | 0.712 | 0.438 | 92 [symbol] |
| 0.011 | 0.295 | 0.026 | 0.267 | 0.085 | 0.261 | 0.600 | 0.563 | 0.421 | 0.591 | 0.376 | 0.594 | 95 [symbol] |

[symbol]

indicates data missing or illegible when filed

TABLE F
Biomarkers identified in F3CSH

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | M/Z |
| P | | P | | P | | P | | P | | P | | ( [symbol] |
| value | ROC | value | ROC | value | ROC | value | ROC | value | ROC | value | ROC | (D [symbol] |
| 0.050 | 0.336 | 0.068 | 0.287 | 0.236 | 0.337 | 0.042 | 0.708 | 0.546 | 0.409 | 0.555 | 0.437 | 1 [symbol] |
| 0.318 | 0.582 | 0.262 | 0.634 | 0.747 | 0.554 | 0.249 | 0.621 | 0.763 | 0.538 | 0.712 | 0.469 | 1 [symbol] |
| 0.404 | 0.602 | 0.118 | 0.634 | 0.518 | 0.446 | 0.950 | 0.486 | 0.088 | 0.758 | 0.941 | 0.542 | 1 [symbol] |
| 0.022 | 0.316 | 0.068 | 0.310 | 0.067 | 0.228 | 0.173 | 0.650 | 0.615 | 0.591 | 0.077 | 0.760 | 1 [symbol] |
| 0.018 | 0.316 | 0.013 | 0.215 | 0.306 | 0.341 | 0.137 | 0.669 | 0.366 | 0.356 | 0.033 | 0.802 | 1 [symbol] |
| 0.019 | 0.295 | 0.016 | 0.219 | 0.282 | 0.337 | 0.090 | 0.698 | 0.269 | 0.318 | 0.005 | 0.885 | 1 [symbol] |
| 0.010 | 0.725 | 0.019 | 0.753 | 0.095 | 0.696 | 0.098 | 0.312 | 0.763 | 0.583 | 0.417 | 0.385 | 2 [symbol] |
| 0.003 | 0.766 | 0.018 | 0.753 | 0.021 | 0.804 | 0.368 | 0.399 | 1.000 | 0.500 | 0.658 | 0.427 | 2 [symbol] |
| 0.001 | 0.786 | 0.005 | 0.800 | 0.021 | 0.804 | 0.020 | 0.225 | 0.920 | 0.492 | 0.883 | 0.542 | 2 [symbol] |
| 0.022 | 0.316 | 0.053 | 0.287 | 0.095 | 0.264 | 0.216 | 0.650 | 0.688 | 0.545 | 0.302 | 0.635 | 2 [symbol] |
| 0.032 | 0.316 | 0.087 | 0.310 | 0.085 | 0.268 | 0.267 | 0.650 | 0.315 | 0.629 | 0.461 | 0.594 | 2 [symbol] |
| 0.011 | 0.254 | 0.024 | 0.263 | 0.095 | 0.268 | 0.249 | 0.630 | 0.841 | 0.530 | 0.658 | 0.563 | [symbol] |
| 0.280 | 0.602 | 0.101 | 0.658 | 0.788 | 0.446 | 0.046 | 0.254 | 0.366 | 0.667 | 0.606 | 0.427 | [symbol] |
| 0.050 | 0.664 | 0.012 | 0.753 | 0.872 | 0.478 | 0.075 | 0.283 | 0.132 | 0.758 | 0.507 | 0.563 | [symbol] |
| 0.129 | 0.623 | 0.031 | 0.706 | 0.872 | 0.482 | 0.249 | 0.341 | 0.070 | 0.803 | 0.507 | 0.604 | [symbol] |
| 0.167 | 0.602 | 0.041 | 0.682 | 0.788 | 0.478 | 0.201 | 0.341 | 0.035 | 0.811 | 0.376 | 0.594 | [symbol] |
| 0.042 | 0.316 | 0.087 | 0.334 | 0.132 | 0.337 | 0.600 | 0.534 | 1.000 | 0.455 | 1.000 | 0.521 | [symbol] |
| 0.065 | 0.377 | 0.049 | 0.314 | 0.451 | 0.409 | 0.267 | 0.621 | 0.688 | 0.455 | 0.185 | 0.271 | [symbol] |
| 0.050 | 0.377 | 0.026 | 0.267 | 0.554 | 0.409 | 0.216 | 0.650 | 0.269 | 0.318 | 0.185 | 0.313 | [symbol] |
| 0.002 | 0.214 | 0.003 | 0.196 | 0.095 | 0.268 | 0.014 | 0.737 | 0.421 | 0.409 | 0.461 | 0.563 | [symbol] |
| 0.010 | 0.746 | 0.026 | 0.729 | 0.067 | 0.732 | 0.600 | 0.428 | 0.688 | 0.402 | 0.825 | 0.438 | [symbol] |
| 0.004 | 0.766 | 0.007 | 0.777 | 0.085 | 0.732 | 0.249 | 0.341 | 0.269 | 0.629 | 0.376 | 0.354 | [symbol] |
| 0.014 | 0.295 | 0.058 | 0.310 | 0.041 | 0.225 | 0.201 | 0.621 | 0.546 | 0.591 | 0.338 | 0.625 | [symbol] |
| 0.151 | 0.602 | 0.179 | 0.634 | 0.389 | 0.623 | 0.850 | 0.514 | 0.615 | 0.591 | 0.941 | 0.479 | 1 [symbol] |
| 0.021 | 0.275 | 0.053 | 0.291 | 0.085 | 0.301 | 0.173 | 0.650 | 0.763 | 0.500 | 0.027 | 0.792 | 1 [symbol] |

[symbol]

indicates data missing or illegible when filed

TABLE G
Biomarkers identified in F5CSL

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | M/Z |
| P | | P | | P | | P | | P | | P | | (avg [symbol] |
| value | ROC | value | ROC | value | ROC | value | ROC | value | ROC | value | ROC | Da |
| 0.397 | 0.540 | 0.320 | 0.580 | 0.836 | 0.500 | 0.903 | 0.493 | 0.366 | 0.621 | 0.000 | 0.031 | 2 [symbol] |
| 0.081 | 0.658 | 0.166 | 0.686 | 0.162 | 0.708 | 0.544 | 0.438 | 0.841 | 0.447 | 0.245 | 0.344 | 2 [symbol] |
| 0.005 | 0.246 | 0.003 | 0.182 | 0.254 | 0.375 | 0.157 | 0.681 | 0.027 | 0.182 | 0.086 | 0.281 | 2 [symbol] |
| 0.034 | 0.678 | 0.030 | 0.712 | 0.325 | 0.583 | 0.039 | 0.243 | 0.482 | 0.621 | 0.298 | 0.625 | 2 [symbol] |
| 0.017 | 0.266 | 0.102 | 0.314 | 0.023 | 0.167 | 0.169 | 0.660 | 0.228 | 0.682 | 0.713 | 0.438 | 2 [symbol] |
| 0.979 | 0.462 | 0.972 | 0.473 | 0.917 | 0.458 | 0.936 | 0.521 | 0.546 | 0.576 | 0.066 | 0.281 | 2 [symbol] |
| 0.015 | 0.266 | 0.036 | 0.235 | 0.087 | 0.292 | 0.008 | 0.819 | 0.841 | 0.538 | 0.221 | 0.375 | 2 [symbol] |
| 0.000 | 0.157 | 0.000 | 0.129 | 0.015 | 0.208 | 0.019 | 0.771 | 0.615 | 0.447 | 0.903 | 0.500 | 2 [symbol] |
| 0.095 | 0.344 | 0.214 | 0.367 | 0.147 | 0.292 | 0.043 | 0.736 | 0.421 | 0.667 | 0.713 | 0.563 | 3 [symbol] |
| 0.053 | 0.364 | 0.065 | 0.341 | 0.276 | 0.333 | 0.075 | 0.701 | 0.546 | 0.447 | 0.076 | 0.281 | 3 [symbol] |
| 0.341 | 0.588 | 0.320 | 0.633 | 0.678 | 0.542 | 0.039 | 0.299 | 0.482 | 0.629 | 0.066 | 0.750 | 3 [symbol] |
| 0.118 | 0.384 | 0.095 | 0.367 | 0.534 | 0.458 | 0.518 | 0.590 | 0.841 | 0.492 | 0.008 | 0.188 | 3 [symbol] |
| 0.009 | 0.266 | 0.005 | 0.208 | 0.300 | 0.375 | 0.146 | 0.674 | 0.035 | 0.182 | 0.501 | 0.406 | 3 [symbol] |
| 0.026 | 0.678 | 0.065 | 0.686 | 0.097 | 0.708 | 0.968 | 0.521 | 0.841 | 0.455 | 0.327 | 0.656 | 3 [symbol] |
| 0.090 | 0.325 | 0.127 | 0.341 | 0.276 | 0.375 | 0.544 | 0.549 | 0.615 | 0.402 | 0.462 | 0.406 | 3 [symbol] |
| 0.427 | 0.403 | 0.696 | 0.447 | 0.325 | 0.333 | 0.467 | 0.569 | 0.615 | 0.591 | 0.668 | 0.563 | 3 [symbol] |
| 0.001 | 0.216 | 0.025 | 0.288 | 0.003 | 0.125 | 0.106 | 0.701 | 0.159 | 0.674 | 0.759 | 0.500 | 3 [symbol] |
| 0.057 | 0.678 | 0.118 | 0.659 | 0.147 | 0.667 | 0.258 | 0.382 | 1.000 | 0.500 | 0.624 | 0.563 | 4 [symbol] |
| 0.011 | 0.737 | 0.013 | 0.765 | 0.178 | 0.667 | 0.006 | 0.188 | 0.841 | 0.538 | 1.000 | 0.531 | 4 [symbol] |
| 0.026 | 0.697 | 0.055 | 0.712 | 0.120 | 0.750 | 0.090 | 0.326 | 0.688 | 0.583 | 0.951 | 0.469 | 4 [symbol] |
| 0.013 | 0.737 | 0.065 | 0.712 | 0.029 | 0.833 | 0.396 | 0.410 | 0.088 | 0.227 | 0.951 | 0.500 | 4 [symbol] |
| 0.017 | 0.678 | 0.001 | 0.818 | 1.000 | 0.500 | 0.312 | 0.410 | 0.035 | 0.803 | 0.713 | 0.531 | 4 [symbol] |
| 0.001 | 0.795 | 0.001 | 0.845 | 0.120 | 0.750 | 0.063 | 0.299 | 0.159 | 0.712 | 0.111 | 0.688 | 4 [symbol] |
| 0.002 | 0.776 | 0.006 | 0.818 | 0.049 | 0.750 | 0.024 | 0.243 | 0.763 | 0.538 | 0.391 | 0.594 | 4 [symbol] |
| 0.032 | 0.697 | 0.095 | 0.686 | 0.078 | 0.750 | 0.196 | 0.354 | 0.763 | 0.409 | 0.951 | 0.500 | 4 [symbol] |
| 0.006 | 0.275 | 0.000 | 0.129 | 0.917 | 0.458 | 0.332 | 0.597 | 0.007 | 0.091 | 0.903 | 0.531 | 4 [symbol] |
| 0.368 | 0.560 | 0.776 | 0.500 | 0.029 | 0.750 | 0.063 | 0.271 | 0.009 | 0.136 | 0.501 | 0.563 | 4 [symbol] |
| 0.017 | 0.737 | 0.145 | 0.659 | 0.011 | 0.833 | 0.052 | 0.299 | 0.035 | 0.182 | 0.501 | 0.594 | 4 [symbol] |
| 0.016 | 0.737 | 0.060 | 0.686 | 0.049 | 0.750 | 0.353 | 0.354 | 0.132 | 0.265 | 0.391 | 0.594 | 4 [symbol] |
| 0.005 | 0.737 | 0.051 | 0.712 | 0.010 | 0.833 | 0.009 | 0.215 | 0.315 | 0.364 | 0.358 | 0.594 | 4 [symbol] |
| 0.090 | 0.638 | 0.256 | 0.633 | 0.097 | 0.708 | 0.716 | 0.438 | 0.191 | 0.318 | 0.713 | 0.563 | 4 [symbol] |
| 0.039 | 0.678 | 0.110 | 0.659 | 0.087 | 0.708 | 0.872 | 0.493 | 0.615 | 0.455 | 0.358 | 0.625 | 4 [symbol] |
| 0.009 | 0.737 | 0.012 | 0.765 | 0.147 | 0.708 | 0.017 | 0.243 | 0.482 | 0.591 | 0.426 | 0.594 | 4 [symbol] |
| 0.023 | 0.678 | 0.047 | 0.712 | 0.120 | 0.708 | 0.312 | 0.382 | 0.841 | 0.500 | 0.951 | 0.500 | 4 [symbol] |
| 0.050 | 0.678 | 0.019 | 0.739 | 0.678 | 0.583 | 0.169 | 0.653 | 0.421 | 0.629 | 0.221 | 0.625 | 4 [symbol] |
| 0.005 | 0.776 | 0.019 | 0.765 | 0.034 | 0.792 | 0.293 | 0.382 | 0.688 | 0.409 | 0.270 | 0.625 | 4 [symbol] |
| 0.014 | 0.697 | 0.002 | 0.792 | 0.756 | 0.542 | 0.599 | 0.438 | 0.056 | 0.803 | 0.759 | 0.438 | [symbol] |
| 0.021 | 0.678 | 0.030 | 0.712 | 0.178 | 0.667 | 0.396 | 0.410 | 0.841 | 0.538 | 0.951 | 0.531 | [symbol] |
| 0.004 | 0.756 | 0.001 | 0.818 | 0.378 | 0.542 | 0.157 | 0.354 | 0.070 | 0.765 | 0.806 | 0.469 | [symbol] |
| 0.001 | 0.216 | 0.001 | 0.182 | 0.133 | 0.333 | 0.903 | 0.486 | 0.108 | 0.273 | 0.668 | 0.469 | [symbol] |
| 0.013 | 0.294 | 0.000 | 0.129 | 0.437 | 0.583 | 0.628 | 0.590 | 0.003 | 0.045 | 0.854 | 0.531 | [symbol] |
| 0.050 | 0.344 | 0.023 | 0.261 | 0.604 | 0.417 | 0.029 | 0.729 | 0.088 | 0.265 | 0.759 | 0.406 | [symbol] |
| 0.000 | 0.157 | 0.000 | 0.102 | 0.043 | 0.250 | 0.026 | 0.764 | 0.159 | 0.318 | 0.951 | 0.500 | [symbol] |
| 0.001 | 0.216 | 0.001 | 0.208 | 0.070 | 0.292 | 0.002 | 0.819 | 0.228 | 0.318 | 0.854 | 0.438 | [symbol] |
| 0.001 | 0.207 | 0.001 | 0.182 | 0.078 | 0.292 | 0.075 | 0.708 | 0.088 | 0.273 | 0.903 | 0.469 | [symbol] |
| 0.000 | 0.207 | 0.001 | 0.155 | 0.070 | 0.250 | 0.069 | 0.687 | 0.056 | 0.227 | 0.903 | 0.500 | [symbol] |
| 0.003 | 0.246 | 0.004 | 0.155 | 0.097 | 0.250 | 0.090 | 0.681 | 0.088 | 0.227 | 0.759 | 0.469 | [symbol] |
| 0.000 | 0.098 | 0.000 | 0.102 | 0.005 | 0.125 | 0.106 | 0.681 | 0.421 | 0.356 | 0.713 | 0.563 | 69 [symbol] |
| 0.004 | 0.776 | 0.076 | 0.633 | 0.002 | 0.917 | 0.442 | 0.438 | 0.108 | 0.273 | 0.270 | 0.594 | 72 [symbol] |
| 0.047 | 0.658 | 0.241 | 0.633 | 0.029 | 0.750 | 0.777 | 0.493 | 0.269 | 0.356 | 0.391 | 0.594 | 7 [symbol] |
| 0.004 | 0.207 | 0.013 | 0.208 | 0.038 | 0.208 | 0.063 | 0.715 | 0.688 | 0.538 | 0.951 | 0.469 | 7 [symbol] |
| 0.032 | 0.314 | 0.118 | 0.314 | 0.055 | 0.250 | 0.312 | 0.597 | 0.421 | 0.621 | 0.582 | 0.406 | 8 [symbol] |
| 0.010 | 0.286 | 0.009 | 0.208 | 0.213 | 0.333 | 0.052 | 0.729 | 0.269 | 0.364 | 0.426 | 0.594 | 8 [symbol] |
| 0.001 | 0.216 | 0.000 | 0.129 | 0.195 | 0.333 | 0.011 | 0.757 | 0.007 | 0.091 | 0.951 | 0.531 | 8 [symbol] |
| 0.023 | 0.325 | 0.025 | 0.314 | 0.233 | 0.375 | 0.014 | 0.764 | 0.688 | 0.455 | 0.759 | 0.531 | 8 [symbol] |
| 0.005 | 0.255 | 0.011 | 0.261 | 0.078 | 0.292 | 0.026 | 0.757 | 0.482 | 0.409 | 0.358 | 0.625 | 9 [symbol] |
| 0.013 | 0.266 | 0.028 | 0.288 | 0.097 | 0.250 | 0.196 | 0.660 | 0.688 | 0.409 | 0.668 | 0.438 | 9 [symbol] |
| 0.081 | 0.678 | 0.065 | 0.712 | 0.468 | 0.583 | 0.374 | 0.410 | 0.366 | 0.621 | 0.951 | 0.500 | 10 [symbol] |
| 0.003 | 0.216 | 0.005 | 0.208 | 0.078 | 0.292 | 0.026 | 0.736 | 0.366 | 0.402 | 0.806 | 0.500 | 13 [symbol] |
| 0.005 | 0.286 | 0.021 | 0.261 | 0.038 | 0.208 | 0.012 | 0.792 | 0.841 | 0.530 | 0.501 | 0.563 | 1 [symbol] |
| 0.204 | 0.364 | 0.859 | 0.473 | 0.026 | 0.208 | 0.052 | 0.708 | 0.016 | 0.856 | 0.854 | 0.531 | 17 [symbol] |
| 0.002 | 0.196 | 0.002 | 0.182 | 0.097 | 0.292 | 0.075 | 0.708 | 0.546 | 0.394 | 0.582 | 0.594 | 21 [symbol] |
| 0.001 | 0.196 | 0.002 | 0.155 | 0.062 | 0.292 | 0.019 | 0.792 | 0.366 | 0.356 | 1.000 | 0.469 | 22 [symbol] |
| 0.002 | 0.196 | 0.002 | 0.155 | 0.133 | 0.292 | 0.010 | 0.764 | 0.315 | 0.318 | 0.713 | 0.438 | 22 [symbol] |
| 0.001 | 0.196 | 0.005 | 0.208 | 0.017 | 0.208 | 0.032 | 0.729 | 0.763 | 0.530 | 0.426 | 0.594 | 25 [symbol] |
| 0.013 | 0.286 | 0.060 | 0.288 | 0.034 | 0.167 | 0.015 | 0.785 | 0.228 | 0.674 | 0.759 | 0.500 | 28 [symbol] |
| 0.019 | 0.275 | 0.095 | 0.288 | 0.029 | 0.167 | 0.036 | 0.736 | 0.159 | 0.720 | 0.540 | 0.563 | 28 [symbol] |
| 0.021 | 0.286 | 0.028 | 0.261 | 0.195 | 0.333 | 0.293 | 0.632 | 0.763 | 0.447 | 0.327 | 0.375 | 33 [symbol] |
| 0.004 | 0.235 | 0.004 | 0.182 | 0.147 | 0.292 | 0.011 | 0.764 | 0.688 | 0.402 | 0.178 | 0.313 | 43 [symbol] |
| 0.003 | 0.235 | 0.002 | 0.182 | 0.213 | 0.333 | 0.011 | 0.806 | 0.228 | 0.311 | 0.111 | 0.281 | 43 [symbol] |
| 0.000 | 0.176 | 0.000 | 0.102 | 0.038 | 0.250 | 0.005 | 0.813 | 0.315 | 0.356 | 0.624 | 0.438 | 44 [symbol] |
| 0.000 | 0.137 | 0.000 | 0.076 | 0.020 | 0.208 | 0.005 | 0.833 | 0.366 | 0.402 | 0.582 | 0.469 | 45 [symbol] |
| 0.000 | 0.176 | 0.000 | 0.129 | 0.070 | 0.250 | 0.008 | 0.813 | 0.920 | 0.447 | 0.159 | 0.281 | 46 [symbol] |
| 0.011 | 0.246 | 0.013 | 0.208 | 0.178 | 0.292 | 0.024 | 0.764 | 0.366 | 0.318 | 0.066 | 0.281 | 47 [symbol] |
| 0.204 | 0.392 | 0.102 | 0.367 | 0.917 | 0.458 | 0.069 | 0.729 | 0.191 | 0.318 | 0.111 | 0.313 | 54 [symbol] |
| 0.026 | 0.314 | 0.036 | 0.288 | 0.195 | 0.333 | 0.048 | 0.729 | 0.920 | 0.492 | 1.000 | 0.469 | 55 [symbol] |
| 0.050 | 0.333 | 0.009 | 0.235 | 1.000 | 0.500 | 0.043 | 0.757 | 0.132 | 0.311 | 0.501 | 0.406 | 57 [symbol] |
| 0.000 | 0.137 | 0.000 | 0.129 | 0.015 | 0.208 | 0.001 | 0.889 | 0.763 | 0.492 | 0.903 | 0.563 | 59 [symbol] |
| 0.000 | 0.196 | 0.000 | 0.155 | 0.070 | 0.250 | 0.002 | 0.861 | 0.615 | 0.447 | 0.624 | 0.438 | 59 [symbol] |
| 0.001 | 0.235 | 0.002 | 0.208 | 0.078 | 0.292 | 0.003 | 0.833 | 0.688 | 0.447 | 0.806 | 0.438 | 60 [symbol] |
| 0.004 | 0.216 | 0.004 | 0.182 | 0.133 | 0.333 | 0.000 | 0.889 | 0.688 | 0.447 | 0.713 | 0.469 | 60 [symbol] |
| 0.002 | 0.235 | 0.006 | 0.208 | 0.049 | 0.250 | 0.003 | 0.833 | 0.841 | 0.492 | 0.462 | 0.375 | 61 [symbol] |
| 0.001 | 0.216 | 0.007 | 0.235 | 0.020 | 0.208 | 0.002 | 0.861 | 0.688 | 0.538 | 0.391 | 0.375 | 62 [symbol] |
| 0.001 | 0.188 | 0.001 | 0.155 | 0.062 | 0.250 | 0.012 | 0.792 | 0.366 | 0.356 | 0.759 | 0.438 | 6 [symbol] |
| 0.001 | 0.235 | 0.001 | 0.182 | 0.097 | 0.292 | 0.001 | 0.889 | 0.615 | 0.492 | 0.298 | 0.375 | 7 [symbol] |
| 0.001 | 0.196 | 0.001 | 0.155 | 0.087 | 0.292 | 0.001 | 0.868 | 0.763 | 0.492 | 0.198 | 0.313 | 7 [symbol] |
| 0.000 | 0.157 | 0.000 | 0.155 | 0.038 | 0.250 | 0.001 | 0.840 | 0.482 | 0.364 | 0.951 | 0.469 | 7 [symbol] |
| 0.266 | 0.412 | 0.887 | 0.473 | 0.049 | 0.250 | 0.036 | 0.736 | 0.132 | 0.765 | 0.759 | 0.531 | 8 [symbol] |
| 0.002 | 0.188 | 0.001 | 0.129 | 0.162 | 0.333 | 0.039 | 0.736 | 0.088 | 0.227 | 0.759 | 0.469 | 8 [symbol] |
| 0.000 | 0.196 | 0.001 | 0.155 | 0.049 | 0.250 | 0.043 | 0.729 | 0.269 | 0.318 | 0.854 | 0.469 | 9 [symbol] |

[symbol]

indicates data missing or illegible when filed

TABLE H
Biomarkers identified in F5CSH

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | M/Z |
| P | | P | | P | | P | | P | | P | | (a [symbol] |
| value | ROC | value | ROC | value | ROC | value | ROC | value | ROC | value | ROC | Da |
| 0.021 | 0.316 | 0.028 | 0.263 | 0.178 | 0.301 | 0.201 | 0.688 | 0.482 | 0.409 | 0.333 | 0.363 | 1 [symbol] |
| 0.047 | 0.316 | 0.087 | 0.330 | 0.162 | 0.301 | 0.057 | 0.708 | 0.920 | 0.545 | 0.651 | 0.563 | 1 [symbol] |
| 0.036 | 0.684 | 0.053 | 0.706 | 0.196 | 0.659 | 0.600 | 0.447 | 0.315 | 0.674 | 0.093 | 0.696 | 1 [symbol] |
| 0.022 | 0.725 | 0.028 | 0.706 | 0.196 | 0.659 | 0.818 | 0.476 | 0.228 | 0.674 | 0.053 | 0.758 | 1 [symbol] |
| 0.030 | 0.705 | 0.038 | 0.729 | 0.216 | 0.696 | 0.285 | 0.370 | 0.421 | 0.629 | 0.561 | 0.563 | 1 [symbol] |
| 0.009 | 0.746 | 0.013 | 0.777 | 0.132 | 0.696 | 0.216 | 0.341 | 0.421 | 0.583 | 1.000 | 0.463 | 1 [symbol] |
| 0.001 | 0.807 | 0.000 | 0.848 | 0.132 | 0.696 | 0.057 | 0.283 | 0.016 | 0.848 | 0.897 | 0.496 | 1 [symbol] |
| 0.013 | 0.684 | 0.038 | 0.706 | 0.067 | 0.696 | 0.038 | 0.283 | 0.763 | 0.447 | 0.747 | 0.529 | 1 [symbol] |
| 0.018 | 0.705 | 0.008 | 0.753 | 0.419 | 0.623 | 0.216 | 0.312 | 0.070 | 0.803 | 0.796 | 0.525 | 1 [symbol] |
| 0.008 | 0.725 | 0.005 | 0.777 | 0.282 | 0.627 | 0.572 | 0.399 | 0.191 | 0.712 | 0.747 | 0.433 | 1 [symbol] |
| 0.005 | 0.746 | 0.011 | 0.777 | 0.067 | 0.732 | 0.075 | 0.283 | 0.841 | 0.538 | 0.846 | 0.558 | 1 [symbol] |
| 0.006 | 0.746 | 0.002 | 0.824 | 0.360 | 0.623 | 0.187 | 0.341 | 0.088 | 0.712 | 0.846 | 0.525 | 1 [symbol] |
| 0.001 | 0.807 | 0.001 | 0.848 | 0.053 | 0.768 | 0.051 | 0.283 | 0.841 | 0.492 | 0.651 | 0.596 | 1 [symbol] |
| 0.017 | 0.746 | 0.021 | 0.753 | 0.178 | 0.696 | 0.098 | 0.283 | 0.366 | 0.629 | 0.699 | 0.529 | 1 [symbol] |
| 0.000 | 0.848 | 0.000 | 0.919 | 0.306 | 0.591 | 0.267 | 0.389 | 0.108 | 0.758 | 0.651 | 0.592 | 1 [symbol] |
| 0.003 | 0.746 | 0.002 | 0.824 | 0.196 | 0.696 | 0.042 | 0.254 | 0.315 | 0.667 | 0.846 | 0.525 | 1 [symbol] |
| 0.003 | 0.766 | 0.001 | 0.824 | 0.216 | 0.696 | 0.042 | 0.225 | 0.228 | 0.674 | 0.846 | 0.463 | 1 [symbol] |
| 0.000 | 0.848 | 0.000 | 0.872 | 0.041 | 0.768 | 0.126 | 0.370 | 0.132 | 0.712 | 0.519 | 0.429 | 1 [symbol] |
| 0.050 | 0.316 | 0.019 | 0.259 | 0.667 | 0.409 | 0.046 | 0.727 | 0.088 | 0.265 | 0.366 | 0.396 | 1 [symbol] |
| 0.002 | 0.214 | 0.003 | 0.172 | 0.060 | 0.264 | 0.107 | 0.650 | 0.366 | 0.356 | 0.245 | 0.658 | 1 [symbol] |
| 0.003 | 0.254 | 0.005 | 0.215 | 0.095 | 0.301 | 0.034 | 0.717 | 0.421 | 0.409 | 0.796 | 0.496 | 1 [symbol] |
| 0.030 | 0.295 | 0.026 | 0.259 | 0.306 | 0.370 | 0.098 | 0.679 | 0.070 | 0.273 | 0.651 | 0.558 | 1 [symbol] |
| 0.104 | 0.643 | 0.026 | 0.729 | 0.957 | 0.514 | 0.722 | 0.428 | 0.009 | 0.894 | 0.197 | 0.658 | 1 [symbol] |
| 0.575 | 0.459 | 0.429 | 0.571 | 0.024 | 0.192 | 0.850 | 0.457 | 0.027 | 0.856 | 0.561 | 0.592 | 1 [symbol] |
| 0.004 | 0.786 | 0.003 | 0.824 | 0.196 | 0.659 | 0.034 | 0.283 | 0.108 | 0.720 | 0.606 | 0.429 | 2 [symbol] |
| 0.021 | 0.254 | 0.008 | 0.235 | 0.518 | 0.409 | 0.267 | 0.630 | 0.108 | 0.265 | 0.478 | 0.592 | 2 [symbol] |
| 0.039 | 0.316 | 0.118 | 0.354 | 0.076 | 0.228 | 0.057 | 0.708 | 0.269 | 0.674 | 0.897 | 0.529 | 2 [symbol] |
| 0.011 | 0.254 | 0.008 | 0.247 | 0.236 | 0.377 | 0.028 | 0.756 | 0.615 | 0.402 | 0.245 | 0.367 | 4 [symbol] |
| 0.000 | 0.152 | 0.000 | 0.105 | 0.041 | 0.232 | 0.005 | 0.795 | 0.763 | 0.447 | 0.699 | 0.500 | 4 [symbol] |
| 0.305 | 0.582 | 0.408 | 0.587 | 0.419 | 0.623 | 0.346 | 0.370 | 0.763 | 0.492 | 0.197 | 0.663 | 4 [symbol] |
| 0.007 | 0.746 | 0.013 | 0.753 | 0.095 | 0.732 | 0.917 | 0.486 | 0.482 | 0.583 | 0.197 | 0.329 | [symbol] |
| 0.001 | 0.786 | 0.003 | 0.800 | 0.053 | 0.768 | 0.489 | 0.428 | 0.841 | 0.538 | 0.366 | 0.396 | [symbol] |
| 0.019 | 0.275 | 0.019 | 0.263 | 0.236 | 0.337 | 0.016 | 0.766 | 0.421 | 0.364 | 0.401 | 0.396 | [symbol] |
| 0.018 | 0.275 | 0.018 | 0.263 | 0.236 | 0.301 | 0.020 | 0.737 | 0.482 | 0.409 | 0.333 | 0.363 | [symbol] |
| 0.001 | 0.193 | 0.000 | 0.148 | 0.106 | 0.268 | 0.022 | 0.795 | 0.421 | 0.402 | 0.796 | 0.429 | [symbol] |
| 0.003 | 0.234 | 0.005 | 0.196 | 0.085 | 0.264 | 0.090 | 0.708 | 0.421 | 0.409 | 0.699 | 0.563 | [symbol] |
| 0.010 | 0.275 | 0.068 | 0.306 | 0.015 | 0.196 | 0.187 | 0.659 | 0.159 | 0.674 | 0.081 | 0.729 | [symbol] |
| 0.213 | 0.357 | 0.985 | 0.540 | 0.015 | 0.159 | 0.630 | 0.572 | 0.002 | 0.939 | 0.081 | 0.725 | [symbol] |
| 0.001 | 0.173 | 0.001 | 0.144 | 0.118 | 0.264 | 0.020 | 0.766 | 0.088 | 0.265 | 0.699 | 0.429 | [symbol] |
| 0.001 | 0.193 | 0.001 | 0.144 | 0.178 | 0.337 | 0.098 | 0.737 | 0.191 | 0.356 | 0.949 | 0.462 | [symbol] |
| 0.001 | 0.193 | 0.001 | 0.148 | 0.067 | 0.232 | 0.137 | 0.659 | 0.132 | 0.318 | 0.366 | 0.363 | 1 [symbol] |
| 0.044 | 0.336 | 0.028 | 0.271 | 0.451 | 0.409 | 0.148 | 0.630 | 0.546 | 0.455 | 0.561 | 0.429 | 1 [symbol] |
| 0.003 | 0.254 | 0.004 | 0.172 | 0.106 | 0.301 | 0.002 | 0.843 | 0.482 | 0.364 | 0.949 | 0.458 | 1 [symbol] |
| 0.012 | 0.275 | 0.013 | 0.239 | 0.196 | 0.337 | 0.034 | 0.727 | 0.228 | 0.318 | 1.000 | 0.492 | 1 [symbol] |
| 0.000 | 0.111 | 0.000 | 0.101 | 0.011 | 0.163 | 0.014 | 0.795 | 0.615 | 0.455 | 0.846 | 0.458 | 1 [symbol] |
| 0.000 | 0.173 | 0.001 | 0.125 | 0.031 | 0.196 | 0.126 | 0.659 | 0.191 | 0.273 | 0.333 | 0.363 | 1 [symbol] |

[symbol]

indicates data missing or illegible when filed

TABLE I
Biomarkers identified in F5ISL

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | M/Z |
| P | | P | | P | | P | | P | | P | | (avg [symbol] |
| value | ROC | value | ROC | value | ROC | value | ROC | value | ROC | value | ROC | Da |
| 0.204 | 0.392 | 0.831 | 0.500 | 0.029 | 0.250 | 0.808 | 0.521 | 0.056 | 0.765 | 0.582 | 0.438 | 2 [symbol] |
| 0.397 | 0.442 | 0.915 | 0.473 | 0.133 | 0.333 | 0.032 | 0.736 | 0.088 | 0.712 | 0.391 | 0.406 | 2 [symbol] |
| 0.112 | 0.638 | 0.271 | 0.606 | 0.133 | 0.708 | 0.012 | 0.236 | 0.366 | 0.356 | 0.806 | 0.469 | 2 [symbol] |
| 0.195 | 0.353 | 0.189 | 0.314 | 0.534 | 0.458 | 1.000 | 0.521 | 0.688 | 0.447 | 0.462 | 0.406 | 2 [symbol] |
| 0.101 | 0.384 | 0.095 | 0.341 | 0.437 | 0.417 | 0.716 | 0.451 | 0.763 | 0.492 | 0.501 | 0.438 | 2 [symbol] |
| 0.223 | 0.353 | 0.303 | 0.367 | 0.378 | 0.375 | 0.135 | 0.674 | 0.615 | 0.409 | 0.003 | 0.125 | 2 [symbol] |
| 0.177 | 0.373 | 0.189 | 0.367 | 0.468 | 0.417 | 0.746 | 0.521 | 1.000 | 0.538 | 0.086 | 0.344 | 2 [symbol] |
| 0.037 | 0.314 | 0.166 | 0.367 | 0.038 | 0.250 | 0.396 | 0.576 | 0.920 | 0.500 | 0.221 | 0.375 | 2 [symbol] |
| 0.223 | 0.373 | 0.241 | 0.394 | 0.500 | 0.417 | 0.210 | 0.646 | 0.546 | 0.447 | 0.540 | 0.438 | 2 [symbol] |
| 0.341 | 0.599 | 0.286 | 0.633 | 0.756 | 0.542 | 0.968 | 0.486 | 0.763 | 0.538 | 0.806 | 0.500 | 2 [symbol] |
| 0.791 | 0.529 | 0.972 | 0.500 | 0.568 | 0.583 | 0.396 | 0.458 | 0.421 | 0.447 | 0.017 | 0.219 | 2 [symbol] |
| 0.278 | 0.403 | 0.166 | 0.341 | 0.917 | 0.458 | 0.032 | 0.736 | 0.269 | 0.318 | 0.462 | 0.594 | 2 [symbol] |
| 0.001 | 0.188 | 0.001 | 0.129 | 0.078 | 0.292 | 0.106 | 0.701 | 0.056 | 0.273 | 0.462 | 0.406 | 2 [symbol] |
| 0.090 | 0.353 | 0.065 | 0.288 | 0.534 | 0.417 | 0.777 | 0.479 | 0.482 | 0.409 | 0.358 | 0.375 | 2 [symbol] |
| 0.578 | 0.549 | 0.619 | 0.527 | 0.717 | 0.542 | 0.808 | 0.542 | 0.841 | 0.485 | 0.032 | 0.219 | 2 [symbol] |
| 0.234 | 0.373 | 0.189 | 0.394 | 0.678 | 0.417 | 0.005 | 0.819 | 0.546 | 0.409 | 0.298 | 0.375 | 2 [symbol] |
| 0.214 | 0.619 | 0.414 | 0.606 | 0.213 | 0.625 | 0.106 | 0.299 | 0.546 | 0.455 | 0.903 | 0.563 | 2 [symbol] |
| 0.004 | 0.235 | 0.011 | 0.261 | 0.062 | 0.292 | 0.746 | 0.486 | 0.920 | 0.492 | 0.462 | 0.406 | 2 [symbol] |
| 0.459 | 0.423 | 0.227 | 0.367 | 0.756 | 0.500 | 0.275 | 0.674 | 0.421 | 0.364 | 0.327 | 0.375 | 2 [symbol] |
| 0.118 | 0.373 | 0.060 | 0.314 | 0.756 | 0.500 | 0.135 | 0.326 | 0.763 | 0.485 | 0.540 | 0.438 | 2 [symbol] |
| 0.354 | 0.580 | 0.943 | 0.500 | 0.055 | 0.750 | 0.374 | 0.424 | 0.421 | 0.409 | 0.142 | 0.313 | 2 [symbol] |
| 0.475 | 0.423 | 0.394 | 0.394 | 0.876 | 0.458 | 0.746 | 0.514 | 0.763 | 0.576 | 0.462 | 0.406 | 2 [symbol] |
| 0.234 | 0.619 | 1.000 | 0.473 | 0.020 | 0.833 | 0.419 | 0.431 | 0.269 | 0.364 | 0.501 | 0.406 | 3 [symbol] |
| 0.874 | 0.521 | 0.722 | 0.447 | 0.407 | 0.583 | 0.075 | 0.701 | 0.269 | 0.318 | 0.032 | 0.219 | 3 [symbol] |
| 0.000 | 0.176 | 0.001 | 0.182 | 0.029 | 0.208 | 0.048 | 0.708 | 0.920 | 0.492 | 0.298 | 0.375 | 3 [symbol] |
| 0.412 | 0.423 | 0.776 | 0.447 | 0.233 | 0.292 | 0.571 | 0.458 | 0.421 | 0.583 | 0.327 | 0.375 | 3 [symbol] |
| 0.214 | 0.373 | 0.166 | 0.367 | 0.678 | 0.458 | 0.872 | 0.486 | 0.366 | 0.364 | 0.391 | 0.656 | 3 [symbol] |
| 0.047 | 0.294 | 0.011 | 0.235 | 0.876 | 0.500 | 0.442 | 0.542 | 0.056 | 0.227 | 0.903 | 0.469 | 3 [symbol] |
| 0.053 | 0.658 | 0.201 | 0.606 | 0.055 | 0.750 | 0.599 | 0.465 | 0.366 | 0.364 | 0.007 | 0.156 | 3 [symbol] |
| 0.068 | 0.353 | 0.136 | 0.394 | 0.162 | 0.333 | 0.442 | 0.590 | 1.000 | 0.500 | 1.000 | 0.500 | 3 [symbol] |
| 0.634 | 0.442 | 0.776 | 0.447 | 0.604 | 0.417 | 0.968 | 0.493 | 0.546 | 0.538 | 0.462 | 0.406 | 3 [symbol] |
| 0.177 | 0.384 | 0.088 | 0.314 | 0.876 | 0.458 | 0.021 | 0.757 | 0.763 | 0.492 | 0.624 | 0.469 | 3 [symbol] |
| 0.081 | 0.333 | 0.177 | 0.341 | 0.147 | 0.292 | 0.135 | 0.674 | 0.366 | 0.667 | 0.624 | 0.594 | 3 [symbol] |
| 0.037 | 0.305 | 0.055 | 0.261 | 0.195 | 0.333 | 0.872 | 0.514 | 0.315 | 0.364 | 0.624 | 0.438 | 3 [symbol] |
| 0.525 | 0.521 | 0.594 | 0.527 | 0.641 | 0.500 | 0.936 | 0.521 | 0.841 | 0.455 | 0.624 | 0.438 | [symbol] |
| 0.002 | 0.776 | 0.016 | 0.739 | 0.010 | 0.833 | 0.048 | 0.271 | 0.366 | 0.364 | 0.540 | 0.594 | [symbol] |
| 0.001 | 0.795 | 0.012 | 0.739 | 0.006 | 0.833 | 0.057 | 0.271 | 0.763 | 0.591 | 0.713 | 0.469 | [symbol] |
| 0.302 | 0.599 | 0.456 | 0.553 | 0.351 | 0.625 | 0.057 | 0.299 | 0.615 | 0.409 | 0.668 | 0.438 | [symbol] |
| 0.131 | 0.619 | 0.356 | 0.633 | 0.108 | 0.667 | 0.657 | 0.438 | 0.315 | 0.318 | 0.426 | 0.625 | [symbol] |
| 0.076 | 0.638 | 0.177 | 0.633 | 0.133 | 0.708 | 0.571 | 0.438 | 0.688 | 0.455 | 0.759 | 0.563 | [symbol] |
| 0.010 | 0.737 | 0.060 | 0.712 | 0.023 | 0.792 | 0.052 | 0.271 | 0.159 | 0.273 | 0.951 | 0.500 | [symbol] |
| 0.397 | 0.580 | 0.155 | 0.633 | 0.678 | 0.417 | 0.374 | 0.410 | 0.070 | 0.765 | 0.076 | 0.750 | [symbol] |
| 0.068 | 0.658 | 0.110 | 0.659 | 0.213 | 0.625 | 0.135 | 0.326 | 0.920 | 0.447 | 0.159 | 0.656 | [symbol] |
| 0.006 | 0.737 | 0.008 | 0.765 | 0.133 | 0.708 | 0.010 | 0.215 | 0.366 | 0.674 | 0.098 | 0.719 | [symbol] |
| 0.016 | 0.678 | 0.088 | 0.686 | 0.026 | 0.792 | 0.009 | 0.215 | 0.228 | 0.318 | 0.221 | 0.625 | [symbol] |
| 0.341 | 0.580 | 0.570 | 0.580 | 0.300 | 0.667 | 0.777 | 0.493 | 0.315 | 0.364 | 0.759 | 0.563 | 4 [symbol] |
| 0.060 | 0.658 | 0.110 | 0.633 | 0.178 | 0.625 | 0.396 | 0.382 | 0.763 | 0.492 | 0.759 | 0.469 | 4 [symbol] |
| 0.050 | 0.678 | 0.047 | 0.686 | 0.351 | 0.625 | 0.903 | 0.493 | 0.841 | 0.538 | 0.426 | 0.375 | 5 [symbol] |
| 0.001 | 0.176 | 0.003 | 0.208 | 0.026 | 0.208 | 0.048 | 0.736 | 0.421 | 0.409 | 0.391 | 0.375 | 8 [symbol] |
| 0.112 | 0.344 | 0.776 | 0.473 | 0.007 | 0.167 | 0.069 | 0.687 | 0.005 | 0.902 | 0.178 | 0.313 | 1 [symbol] |
| 0.008 | 0.286 | 0.055 | 0.261 | 0.017 | 0.167 | 0.069 | 0.687 | 0.088 | 0.758 | 0.903 | 0.469 | 33 [symbol] |
| 0.010 | 0.266 | 0.043 | 0.261 | 0.038 | 0.208 | 0.293 | 0.576 | 0.159 | 0.667 | 0.540 | 0.406 | 34 [symbol] |
| 0.021 | 0.305 | 0.082 | 0.367 | 0.049 | 0.208 | 0.312 | 0.604 | 0.159 | 0.674 | 0.391 | 0.375 | 34 [symbol] |
| 0.028 | 0.294 | 0.214 | 0.341 | 0.013 | 0.167 | 0.419 | 0.597 | 0.056 | 0.758 | 0.462 | 0.406 | 35 [symbol] |
| 0.003 | 0.246 | 0.009 | 0.208 | 0.049 | 0.208 | 0.146 | 0.604 | 0.841 | 0.530 | 1.000 | 0.469 | 36 [symbol] |
| 0.578 | 0.451 | 0.831 | 0.473 | 0.437 | 0.417 | 0.196 | 0.674 | 0.546 | 0.576 | 0.624 | 0.438 | 42 [symbol] |
| 0.015 | 0.266 | 0.110 | 0.314 | 0.015 | 0.167 | 0.057 | 0.715 | 0.070 | 0.803 | 0.668 | 0.531 | 42 [symbol] |
| 0.050 | 0.333 | 0.414 | 0.420 | 0.008 | 0.167 | 0.125 | 0.674 | 0.044 | 0.811 | 0.582 | 0.438 | 43 [symbol] |
| 0.042 | 0.325 | 0.051 | 0.314 | 0.254 | 0.333 | 0.275 | 0.632 | 0.920 | 0.492 | 0.624 | 0.438 | 44 [symbol] |
| 0.003 | 0.227 | 0.036 | 0.288 | 0.004 | 0.125 | 0.057 | 0.708 | 0.159 | 0.720 | 0.391 | 0.438 | 44 [symbol] |
| 0.034 | 0.325 | 0.320 | 0.447 | 0.007 | 0.167 | 0.012 | 0.764 | 0.035 | 0.856 | 0.582 | 0.438 | 45 [symbol] |
| 0.003 | 0.207 | 0.009 | 0.208 | 0.049 | 0.208 | 0.015 | 0.764 | 0.615 | 0.583 | 1.000 | 0.531 | 46 [symbol] |
| 0.015 | 0.266 | 0.070 | 0.341 | 0.034 | 0.208 | 0.057 | 0.674 | 0.228 | 0.674 | 0.903 | 0.500 | 46 [symbol] |
| 0.010 | 0.235 | 0.047 | 0.261 | 0.029 | 0.208 | 0.024 | 0.743 | 1.000 | 0.492 | 0.540 | 0.594 | 47 [symbol] |
| 0.234 | 0.384 | 0.915 | 0.527 | 0.013 | 0.208 | 0.936 | 0.458 | 0.027 | 0.856 | 0.854 | 0.469 | 58 [symbol] |
| 0.006 | 0.266 | 0.028 | 0.261 | 0.029 | 0.250 | 0.090 | 0.660 | 0.159 | 0.674 | 0.759 | 0.438 | 66 [symbol] |

[symbol]

indicates data missing or illegible when filed

TABLE J
Biomarkers identified in F5ISH

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | M/Z |
| P | | P | | P | | P | | P | | P | | ( [symbol] |
| value | ROC | value | ROC | value | ROC | value | ROC | value | ROC | value | ROC | D [symbol] |
| 0.007 | 0.258 | 0.018 | 0.252 | 0.065 | 0.303 | 0.302 | 0.636 | 1.000 | 0.455 | 0.133 | 0.705 | 1 [symbol] |
| 0.005 | 0.258 | 0.013 | 0.227 | 0.057 | 0.303 | 0.133 | 0.739 | 0.688 | 0.409 | 0.219 | 0.661 | 1 [symbol] |
| 0.019 | 0.295 | 0.051 | 0.295 | 0.073 | 0.303 | 0.061 | 0.705 | 0.763 | 0.455 | 0.152 | 0.696 | 1 [symbol] |
| 0.009 | 0.258 | 0.027 | 0.271 | 0.057 | 0.265 | 0.260 | 0.670 | 0.841 | 0.492 | 0.585 | 0.589 | 1 [symbol] |
| 0.008 | 0.277 | 0.018 | 0.271 | 0.083 | 0.273 | 0.260 | 0.670 | 0.920 | 0.447 | 0.838 | 0.554 | 1 [symbol] |
| 0.012 | 0.258 | 0.027 | 0.252 | 0.083 | 0.303 | 0.425 | 0.602 | 0.763 | 0.447 | 0.585 | 0.598 | 1 [symbol] |
| 0.014 | 0.262 | 0.043 | 0.277 | 0.057 | 0.273 | 0.075 | 0.739 | 0.920 | 0.492 | 0.891 | 0.527 | [symbol] |
| 0.007 | 0.262 | 0.022 | 0.252 | 0.044 | 0.235 | 0.044 | 0.773 | 0.763 | 0.538 | 0.785 | 0.563 | [symbol] |
| 0.005 | 0.243 | 0.016 | 0.252 | 0.044 | 0.197 | 0.083 | 0.705 | 0.920 | 0.455 | 0.785 | 0.562 | [symbol] |
| 0.020 | 0.299 | 0.047 | 0.320 | 0.093 | 0.273 | 0.241 | 0.670 | 1.000 | 0.485 | 0.172 | 0.705 | [symbol] |
| 0.044 | 0.318 | 0.093 | 0.302 | 0.131 | 0.311 | 0.133 | 0.705 | 0.920 | 0.500 | 0.246 | 0.670 | [symbol] |
| 0.047 | 0.337 | 0.136 | 0.326 | 0.083 | 0.235 | 0.159 | 0.670 | 0.615 | 0.538 | 0.495 | 0.598 | [symbol] |
| 0.089 | 0.337 | 0.340 | 0.401 | 0.050 | 0.235 | 0.122 | 0.670 | 0.228 | 0.629 | 0.172 | 0.670 | [symbol] |
| 0.066 | 0.337 | 0.208 | 0.351 | 0.073 | 0.235 | 0.122 | 0.705 | 0.482 | 0.583 | 0.946 | 0.491 | [symbol] |
| 0.054 | 0.322 | 0.117 | 0.357 | 0.131 | 0.280 | 0.039 | 0.739 | 0.763 | 0.538 | 0.891 | 0.527 | [symbol] |
| 0.044 | 0.303 | 0.158 | 0.333 | 0.057 | 0.280 | 0.302 | 0.602 | 0.482 | 0.629 | 0.838 | 0.491 | [symbol] |
| 0.095 | 0.360 | 0.321 | 0.426 | 0.065 | 0.242 | 0.035 | 0.739 | 0.366 | 0.629 | 0.495 | 0.429 | [symbol] |
| 0.100 | 0.651 | 0.086 | 0.692 | 0.467 | 0.576 | 0.963 | 0.500 | 0.421 | 0.583 | 0.246 | 0.357 | [symbol] |
| 0.034 | 0.722 | 0.127 | 0.667 | 0.050 | 0.758 | 0.061 | 0.295 | 0.546 | 0.409 | 0.495 | 0.420 | 1 [symbol] |
| 0.149 | 0.610 | 0.340 | 0.568 | 0.145 | 0.682 | 0.888 | 0.432 | 0.688 | 0.447 | 0.785 | 0.491 | 1 [symbol] |
| 0.100 | 0.647 | 0.181 | 0.643 | 0.198 | 0.682 | 0.302 | 0.364 | 0.841 | 0.492 | 0.946 | 0.500 | 1 [symbol] |
| 0.100 | 0.666 | 0.321 | 0.593 | 0.073 | 0.720 | 0.453 | 0.398 | 0.159 | 0.265 | 0.946 | 0.500 | 1 [symbol] |
| 0.001 | 0.797 | 0.004 | 0.791 | 0.038 | 0.795 | 0.067 | 0.261 | 0.688 | 0.538 | 0.785 | 0.563 | 1 [symbol] |
| 0.017 | 0.703 | 0.039 | 0.692 | 0.093 | 0.682 | 0.189 | 0.364 | 0.763 | 0.455 | 0.539 | 0.598 | 1 [symbol] |
| 0.002 | 0.797 | 0.003 | 0.816 | 0.093 | 0.720 | 0.044 | 0.227 | 0.366 | 0.629 | 0.785 | 0.536 | 1 [symbol] |
| 0.062 | 0.684 | 0.222 | 0.618 | 0.057 | 0.795 | 0.281 | 0.398 | 0.269 | 0.273 | 0.339 | 0.634 | 1 [symbol] |
| 0.003 | 0.797 | 0.016 | 0.742 | 0.019 | 0.795 | 0.091 | 0.295 | 0.421 | 0.356 | 0.838 | 0.527 | 1 [symbol] |
| 0.066 | 0.684 | 0.158 | 0.643 | 0.117 | 0.720 | 0.260 | 0.364 | 0.315 | 0.356 | 0.682 | 0.455 | 1 [symbol] |
| 0.017 | 0.703 | 0.043 | 0.717 | 0.083 | 0.720 | 0.101 | 0.295 | 0.482 | 0.356 | 0.733 | 0.455 | 1 [symbol] |
| 0.017 | 0.741 | 0.079 | 0.667 | 0.033 | 0.795 | 0.101 | 0.330 | 0.191 | 0.311 | 0.275 | 0.670 | 1 [symbol] |
| 0.004 | 0.778 | 0.015 | 0.742 | 0.033 | 0.795 | 0.189 | 0.330 | 0.615 | 0.447 | 0.682 | 0.563 | 1 [symbol] |
| 0.022 | 0.703 | 0.027 | 0.717 | 0.198 | 0.644 | 0.001 | 0.125 | 0.228 | 0.682 | 0.539 | 0.598 | 1 [symbol] |
| 0.002 | 0.778 | 0.002 | 0.816 | 0.104 | 0.720 | 0.091 | 0.261 | 0.191 | 0.667 | 0.891 | 0.527 | 1 [symbol] |
| 0.002 | 0.797 | 0.012 | 0.767 | 0.016 | 0.795 | 0.035 | 0.261 | 0.615 | 0.409 | 0.413 | 0.634 | 1 [symbol] |
| 0.023 | 0.722 | 0.073 | 0.667 | 0.065 | 0.758 | 0.159 | 0.295 | 0.615 | 0.409 | 0.682 | 0.563 | 1 [symbol] |
| 0.010 | 0.741 | 0.008 | 0.767 | 0.218 | 0.644 | 0.061 | 0.295 | 0.482 | 0.621 | 0.682 | 0.563 | 1 [symbol] |
| 0.009 | 0.741 | 0.020 | 0.717 | 0.083 | 0.720 | 0.189 | 0.330 | 0.688 | 0.402 | 0.891 | 0.491 | 1 [symbol] |
| 0.019 | 0.703 | 0.032 | 0.717 | 0.131 | 0.720 | 0.111 | 0.330 | 0.920 | 0.492 | 0.785 | 0.563 | 1 [symbol] |
| 0.010 | 0.722 | 0.020 | 0.742 | 0.093 | 0.720 | 0.061 | 0.261 | 0.421 | 0.636 | 0.946 | 0.563 | 1 [symbol] |
| 0.089 | 0.666 | 0.236 | 0.618 | 0.104 | 0.758 | 0.281 | 0.364 | 0.228 | 0.318 | 0.585 | 0.598 | 1 [symbol] |
| 0.004 | 0.778 | 0.073 | 0.643 | 0.002 | 0.909 | 0.044 | 0.295 | 0.035 | 0.227 | 0.733 | 0.563 | 1 [symbol] |
| 0.047 | 0.684 | 0.194 | 0.593 | 0.044 | 0.795 | 0.007 | 0.159 | 0.482 | 0.409 | 0.585 | 0.455 | 1 [symbol] |
| 0.079 | 0.666 | 0.208 | 0.618 | 0.104 | 0.720 | 0.325 | 0.398 | 0.688 | 0.455 | 0.838 | 0.527 | 1 [symbol] |
| 0.054 | 0.647 | 0.169 | 0.593 | 0.073 | 0.720 | 0.022 | 0.261 | 0.269 | 0.364 | 1.000 | 0.527 | 1 [symbol] |
| 0.058 | 0.666 | 0.222 | 0.618 | 0.050 | 0.795 | 0.044 | 0.261 | 0.269 | 0.318 | 0.785 | 0.563 | 1 [symbol] |
| 0.003 | 0.759 | 0.032 | 0.742 | 0.006 | 0.833 | 0.159 | 0.364 | 0.269 | 0.318 | 0.495 | 0.455 | 1 [symbol] |
| 0.002 | 0.797 | 0.024 | 0.742 | 0.005 | 0.871 | 0.031 | 0.261 | 0.366 | 0.409 | 0.585 | 0.420 | 1 [symbol] |
| 0.047 | 0.684 | 0.136 | 0.643 | 0.083 | 0.758 | 0.075 | 0.261 | 0.920 | 0.455 | 0.585 | 0.563 | 1 [symbol] |
| 0.149 | 0.651 | 0.731 | 0.525 | 0.019 | 0.795 | 0.373 | 0.398 | 0.108 | 0.227 | 0.306 | 0.598 | 1 [symbol] |
| 0.036 | 0.726 | 0.252 | 0.599 | 0.014 | 0.833 | 0.425 | 0.432 | 0.315 | 0.318 | 0.306 | 0.420 | 1 [symbol] |
| 0.193 | 0.618 | 0.760 | 0.531 | 0.033 | 0.795 | 0.111 | 0.330 | 0.108 | 0.273 | 0.946 | 0.491 | 1 [symbol] |
| 0.001 | 0.820 | 0.006 | 0.773 | 0.006 | 0.871 | 1.000 | 0.534 | 0.482 | 0.591 | 0.495 | 0.563 | 1 [symbol] |
| 0.119 | 0.632 | 0.647 | 0.574 | 0.016 | 0.833 | 0.111 | 0.330 | 0.035 | 0.136 | 0.246 | 0.634 | 1 [symbol] |
| 0.066 | 0.674 | 0.302 | 0.605 | 0.033 | 0.795 | 0.174 | 0.364 | 0.228 | 0.318 | 0.275 | 0.634 | 1 [symbol] |
| 0.036 | 0.689 | 0.194 | 0.624 | 0.025 | 0.758 | 0.122 | 0.295 | 0.088 | 0.227 | 0.733 | 0.536 | 1 [symbol] |
| 0.066 | 0.670 | 0.117 | 0.674 | 0.179 | 0.682 | 0.174 | 0.364 | 0.920 | 0.500 | 0.733 | 0.491 | 1 [symbol] |
| 0.223 | 0.614 | 0.194 | 0.667 | 0.614 | 0.568 | 0.888 | 0.568 | 0.615 | 0.545 | 0.891 | 0.527 | 1 [symbol] |
| 0.380 | 0.595 | 0.703 | 0.525 | 0.240 | 0.644 | 0.453 | 0.398 | 0.191 | 0.318 | 0.453 | 0.589 | 1 [symbol] |
| 0.119 | 0.360 | 0.032 | 0.283 | 0.955 | 0.538 | 0.281 | 0.636 | 0.088 | 0.265 | 0.539 | 0.384 | 1 [symbol] |
| 0.039 | 0.303 | 0.027 | 0.258 | 0.401 | 0.386 | 0.348 | 0.602 | 0.088 | 0.227 | 0.172 | 0.705 | [symbol] |
| 0.012 | 0.281 | 0.027 | 0.277 | 0.083 | 0.273 | 0.260 | 0.602 | 0.615 | 0.409 | 0.682 | 0.455 | [symbol] |
| 0.019 | 0.262 | 0.009 | 0.227 | 0.401 | 0.386 | 0.302 | 0.602 | 0.088 | 0.273 | 0.585 | 0.420 | [symbol] |
| 0.799 | 0.524 | 0.380 | 0.407 | 0.073 | 0.758 | 0.453 | 0.432 | 0.044 | 0.182 | 0.785 | 0.491 | [symbol] |
| 0.005 | 0.229 | 0.007 | 0.209 | 0.104 | 0.311 | 0.039 | 0.739 | 0.546 | 0.409 | 0.152 | 0.321 | [symbol] |
| 0.023 | 0.285 | 0.024 | 0.233 | 0.240 | 0.348 | 0.159 | 0.670 | 0.546 | 0.402 | 0.076 | 0.286 | [symbol] |
| 0.001 | 0.176 | 0.002 | 0.165 | 0.044 | 0.242 | 0.009 | 0.807 | 0.546 | 0.409 | 0.041 | 0.250 | [symbol] |
| 0.023 | 0.266 | 0.027 | 0.233 | 0.218 | 0.348 | 0.639 | 0.568 | 0.421 | 0.364 | 0.048 | 0.214 | [symbol] |
| 0.003 | 0.243 | 0.009 | 0.233 | 0.044 | 0.197 | 0.348 | 0.602 | 0.841 | 0.500 | 0.012 | 0.179 | [symbol] |
| 0.034 | 0.707 | 0.101 | 0.649 | 0.073 | 0.758 | 0.028 | 0.261 | 0.159 | 0.311 | 0.838 | 0.563 | [symbol] |
| 0.022 | 0.703 | 0.127 | 0.692 | 0.022 | 0.833 | 0.511 | 0.466 | 0.088 | 0.273 | 0.539 | 0.563 | 4 [symbol] |
| 0.001 | 0.816 | 0.004 | 0.816 | 0.014 | 0.795 | 0.049 | 0.227 | 0.763 | 0.500 | 0.339 | 0.634 | 5 [symbol] |
| 0.004 | 0.759 | 0.005 | 0.791 | 0.093 | 0.758 | 0.049 | 0.295 | 0.615 | 0.576 | 0.838 | 0.527 | 5 [symbol] |
| 0.002 | 0.759 | 0.002 | 0.791 | 0.117 | 0.720 | 0.044 | 0.261 | 0.366 | 0.629 | 0.946 | 0.491 | 5 [symbol] |
| 0.000 | 0.853 | 0.002 | 0.816 | 0.007 | 0.871 | 0.055 | 0.261 | 0.920 | 0.447 | 0.733 | 0.563 | 5 [symbol] |
| 0.001 | 0.816 | 0.003 | 0.816 | 0.029 | 0.795 | 0.146 | 0.330 | 0.482 | 0.629 | 0.838 | 0.527 | 5 [symbol] |
| 0.025 | 0.722 | 0.079 | 0.667 | 0.065 | 0.720 | 0.111 | 0.364 | 0.615 | 0.455 | 0.946 | 0.491 | 5 [symbol] |
| 0.133 | 0.666 | 0.359 | 0.593 | 0.104 | 0.720 | 0.159 | 0.330 | 0.228 | 0.356 | 0.891 | 0.536 | 5 [symbol] |
| 0.044 | 0.707 | 0.285 | 0.599 | 0.016 | 0.833 | 0.542 | 0.364 | 0.228 | 0.318 | 0.152 | 0.634 | 5 [symbol] |
| 0.023 | 0.703 | 0.029 | 0.717 | 0.198 | 0.682 | 0.673 | 0.500 | 0.688 | 0.538 | 0.495 | 0.420 | 5 [symbol] |
| 0.017 | 0.281 | 0.051 | 0.295 | 0.065 | 0.242 | 0.302 | 0.636 | 0.763 | 0.530 | 0.539 | 0.384 | 6 [symbol] |
| 0.054 | 0.318 | 0.093 | 0.326 | 0.179 | 0.348 | 0.482 | 0.568 | 0.763 | 0.538 | 0.101 | 0.286 | 7 [symbol] |
| 0.193 | 0.389 | 0.147 | 0.326 | 0.654 | 0.455 | 0.133 | 0.705 | 0.546 | 0.402 | 1.000 | 0.455 | 8 [symbol] |
| 0.223 | 0.614 | 0.703 | 0.525 | 0.065 | 0.758 | 0.453 | 0.568 | 0.044 | 0.182 | 0.219 | 0.348 | 8 [symbol] |
| 0.106 | 0.647 | 0.359 | 0.593 | 0.065 | 0.720 | 0.373 | 0.568 | 0.228 | 0.318 | 0.375 | 0.375 | 11 [symbol] |
| 0.000 | 0.154 | 0.002 | 0.153 | 0.007 | 0.129 | 0.017 | 0.841 | 0.841 | 0.538 | 0.785 | 0.482 | 13 [symbol] |

[symbol]

indicates data missing or illegible when filed

TABLE K
Biomarkers identified in F6CSL

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | M/ [symbol] |
| P | | P | | P | | P | | P | | P | | (av [symbol] |
| value | ROC | value | ROC | value | ROC | value | ROC | value | ROC | value | ROC | D [symbol] |
| 0.132 | 0.668 | 0.127 | 0.667 | 0.492 | 0.591 | 0.459 | 0.404 | 0.777 | 0.564 | 0.682 | 0.464 | 2 [symbol] |
| 0.359 | 0.568 | 0.541 | 0.568 | 0.349 | 0.636 | 0.296 | 0.417 | 0.777 | 0.509 | 0.682 | 0.429 | 2 [symbol] |
| 0.007 | 0.733 | 0.008 | 0.773 | 0.170 | 0.727 | 0.037 | 0.265 | 0.336 | 0.673 | 0.682 | 0.527 | 2 [symbol] |
| 0.036 | 0.668 | 0.047 | 0.717 | 0.236 | 0.682 | 0.514 | 0.386 | 0.610 | 0.564 | 0.306 | 0.634 | 2 [symbol] |
| 0.020 | 0.688 | 0.067 | 0.692 | 0.053 | 0.727 | 0.041 | 0.295 | 0.777 | 0.455 | 0.306 | 0.670 | 2 [symbol] |
| 0.156 | 0.628 | 0.127 | 0.643 | 0.618 | 0.591 | 0.828 | 0.477 | 0.282 | 0.655 | 0.339 | 0.625 | [symbol] |
| 0.033 | 0.707 | 0.029 | 0.717 | 0.349 | 0.636 | 0.361 | 0.386 | 0.193 | 0.673 | 0.633 | 0.429 | [symbol] |
| 0.002 | 0.222 | 0.005 | 0.209 | 0.061 | 0.227 | 0.192 | 0.720 | 0.777 | 0.509 | 0.585 | 0.420 | [symbol] |
| 0.287 | 0.588 | 0.236 | 0.618 | 0.755 | 0.545 | 0.695 | 0.447 | 0.865 | 0.509 | 0.785 | 0.527 | [symbol] |
| 0.051 | 0.707 | 0.024 | 0.767 | 0.662 | 0.545 | 0.164 | 0.326 | 0.157 | 0.727 | 0.306 | 0.598 | [symbol] |
| 0.005 | 0.773 | 0.009 | 0.773 | 0.092 | 0.682 | 0.338 | 0.386 | 0.462 | 0.618 | 0.413 | 0.563 | [symbol] |
| 0.010 | 0.727 | 0.039 | 0.717 | 0.039 | 0.818 | 0.056 | 0.295 | 0.692 | 0.564 | 0.733 | 0.420 | [symbol] |
| 0.001 | 0.827 | 0.002 | 0.816 | 0.029 | 0.818 | 0.013 | 0.235 | 0.193 | 0.673 | 0.946 | 0.527 | [symbol] |
| 0.002 | 0.807 | 0.001 | 0.890 | 0.318 | 0.636 | 0.019 | 0.265 | 0.126 | 0.764 | 0.838 | 0.527 | [symbol] |
| 0.033 | 0.693 | 0.002 | 0.816 | 0.618 | 0.455 | 0.408 | 0.629 | 0.079 | 0.764 | 0.306 | 0.357 | [symbol] |
| 0.007 | 0.767 | 0.001 | 0.866 | 0.950 | 0.500 | 0.542 | 0.447 | 0.062 | 0.818 | 0.246 | 0.321 | [symbol] |
| 0.012 | 0.747 | 0.016 | 0.767 | 0.170 | 0.727 | 0.024 | 0.235 | 0.282 | 0.673 | 0.495 | 0.429 | [symbol] |
| 0.036 | 0.688 | 0.047 | 0.692 | 0.236 | 0.682 | 0.050 | 0.295 | 0.533 | 0.618 | 0.891 | 0.491 | [symbol] |
| 0.001 | 0.787 | 0.001 | 0.866 | 0.261 | 0.636 | 0.061 | 0.265 | 0.027 | 0.873 | 0.785 | 0.527 | [symbol] |
| 0.002 | 0.787 | 0.000 | 0.866 | 0.574 | 0.636 | 0.128 | 0.326 | 0.011 | 0.927 | 0.785 | 0.429 | [symbol] |
| 0.051 | 0.315 | 0.027 | 0.277 | 0.618 | 0.455 | 0.486 | 0.616 | 0.396 | 0.345 | 0.785 | 0.429 | [symbol] |
| 0.018 | 0.281 | 0.047 | 0.302 | 0.081 | 0.241 | 0.177 | 0.677 | 0.533 | 0.564 | 0.306 | 0.661 | [symbol] |
| 0.081 | 0.688 | 0.018 | 0.791 | 0.851 | 0.455 | 0.632 | 0.447 | 0.011 | 0.873 | 0.133 | 0.348 | 4 [symbol] |
| 0.261 | 0.628 | 0.043 | 0.742 | 0.349 | 0.318 | 0.338 | 0.356 | 0.011 | 0.873 | 0.585 | 0.393 | 5 [symbol] |
| 0.000 | 0.182 | 0.000 | 0.134 | 0.034 | 0.195 | 0.045 | 0.720 | 0.157 | 0.291 | 0.838 | 0.455 | 6 [symbol] |
| 0.001 | 0.202 | 0.001 | 0.159 | 0.081 | 0.227 | 0.030 | 0.720 | 0.157 | 0.291 | 0.339 | 0.357 | 6 [symbol] |
| 0.001 | 0.202 | 0.002 | 0.134 | 0.081 | 0.273 | 0.117 | 0.659 | 0.100 | 0.182 | 0.785 | 0.455 | 6 [symbol] |
| 0.001 | 0.176 | 0.001 | 0.128 | 0.081 | 0.227 | 0.045 | 0.720 | 0.020 | 0.127 | 0.453 | 0.384 | 6 [symbol] |
| 0.001 | 0.182 | 0.001 | 0.134 | 0.070 | 0.227 | 0.068 | 0.689 | 0.193 | 0.291 | 0.733 | 0.420 | 6 [symbol] |
| 0.044 | 0.301 | 0.029 | 0.258 | 0.492 | 0.377 | 0.017 | 0.750 | 0.126 | 0.273 | 0.453 | 0.384 | 6 [symbol] |
| 0.287 | 0.395 | 0.147 | 0.376 | 0.901 | 0.545 | 0.107 | 0.689 | 0.004 | 0.018 | 0.306 | 0.393 | 7 [symbol] |
| 0.031 | 0.713 | 0.016 | 0.742 | 0.533 | 0.605 | 0.602 | 0.434 | 0.336 | 0.655 | 0.838 | 0.491 | 8 [symbol] |
| 0.013 | 0.281 | 0.061 | 0.308 | 0.029 | 0.195 | 0.663 | 0.568 | 0.193 | 0.673 | 0.020 | 0.768 | 8 [symbol] |
| 0.005 | 0.247 | 0.015 | 0.283 | 0.061 | 0.241 | 0.207 | 0.646 | 0.955 | 0.509 | 0.133 | 0.705 | 8 [symbol] |
| 0.001 | 0.182 | 0.002 | 0.202 | 0.034 | 0.195 | 0.107 | 0.689 | 0.336 | 0.345 | 0.539 | 0.589 | 8 [symbol] |
| 0.001 | 0.182 | 0.001 | 0.134 | 0.119 | 0.286 | 0.030 | 0.720 | 0.126 | 0.236 | 0.413 | 0.625 | 8 [symbol] |
| 0.002 | 0.216 | 0.002 | 0.178 | 0.170 | 0.318 | 0.008 | 0.811 | 0.100 | 0.236 | 0.633 | 0.589 | 8 [symbol] |
| 0.009 | 0.256 | 0.013 | 0.252 | 0.151 | 0.273 | 0.019 | 0.780 | 0.533 | 0.455 | 0.633 | 0.554 | 9 [symbol] |
| 0.011 | 0.241 | 0.022 | 0.258 | 0.105 | 0.286 | 0.050 | 0.720 | 0.865 | 0.509 | 0.585 | 0.589 | 9 [symbol] |
| 0.014 | 0.256 | 0.027 | 0.227 | 0.119 | 0.241 | 0.009 | 0.811 | 0.955 | 0.491 | 0.453 | 0.554 | 9 [symbol] |
| 0.029 | 0.276 | 0.051 | 0.302 | 0.151 | 0.273 | 0.001 | 0.871 | 0.865 | 0.509 | 1.000 | 0.455 | 9 [symbol] |
| 0.018 | 0.727 | 0.015 | 0.767 | 0.318 | 0.636 | 0.177 | 0.356 | 0.396 | 0.600 | 0.219 | 0.357 | 11 [symbol] |
| 0.027 | 0.688 | 0.027 | 0.742 | 0.289 | 0.636 | 0.107 | 0.295 | 0.282 | 0.655 | 0.539 | 0.429 | 11 [symbol] |
| 0.007 | 0.256 | 0.006 | 0.202 | 0.236 | 0.318 | 0.061 | 0.720 | 0.126 | 0.291 | 0.838 | 0.527 | 13 [symbol] |
| 0.021 | 0.281 | 0.036 | 0.283 | 0.151 | 0.318 | 0.041 | 0.689 | 0.955 | 0.509 | 0.682 | 0.429 | 14 [symbol] |
| 0.004 | 0.256 | 0.043 | 0.252 | 0.005 | 0.105 | 0.695 | 0.495 | 0.047 | 0.818 | 0.375 | 0.598 | 15 [symbol] |
| 0.005 | 0.227 | 0.020 | 0.240 | 0.039 | 0.195 | 0.128 | 0.677 | 0.955 | 0.509 | 0.891 | 0.491 | 17 [symbol] |
| 0.009 | 0.276 | 0.005 | 0.202 | 0.349 | 0.364 | 0.026 | 0.737 | 0.047 | 0.236 | 0.495 | 0.429 | 17 [symbol] |
| 0.007 | 0.227 | 0.009 | 0.215 | 0.151 | 0.332 | 0.050 | 0.737 | 0.336 | 0.345 | 0.585 | 0.491 | 17 [symbol] |
| 0.003 | 0.767 | 0.002 | 0.816 | 0.170 | 0.727 | 0.896 | 0.538 | 0.126 | 0.727 | 0.076 | 0.286 | 21 [symbol] |
| 0.086 | 0.668 | 0.194 | 0.643 | 0.134 | 0.727 | 0.862 | 0.508 | 0.336 | 0.345 | 0.034 | 0.250 | 2 [symbol] |
| 0.005 | 0.747 | 0.007 | 0.791 | 0.119 | 0.682 | 0.572 | 0.386 | 0.610 | 0.564 | 0.633 | 0.589 | 2 [symbol] |
| 0.033 | 0.301 | 0.067 | 0.333 | 0.134 | 0.286 | 0.010 | 0.798 | 0.955 | 0.509 | 0.339 | 0.393 | 2 [symbol] |
| 0.033 | 0.321 | 0.022 | 0.283 | 0.454 | 0.409 | 0.013 | 0.768 | 0.193 | 0.291 | 0.076 | 0.250 | 4 [symbol] |
| 0.071 | 0.688 | 0.181 | 0.643 | 0.105 | 0.773 | 0.602 | 0.586 | 0.396 | 0.345 | 0.024 | 0.214 | 5 [symbol] |
| 0.005 | 0.767 | 0.022 | 0.717 | 0.029 | 0.818 | 0.794 | 0.447 | 0.462 | 0.400 | 0.024 | 0.214 | 5 [symbol] |

[symbol]

indicates data missing or illegible when filed

TABLE L
Biomarkers identified in F6CSH

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | |
| P | | P | | P | | P | | P | | P | | M/Z [symbol] |
| value | ROC | value | ROC | value | ROC | value | ROC | value | ROC | value | ROC | D [symbol] |
| 0.006 | 0.251 | 0.067 | 0.314 | 0.006 | 0.167 | 0.024 | 0.768 | 0.546 | 0.636 | 0.682 | 0.429 | [symbol] |
| 0.003 | 0.233 | 0.036 | 0.264 | 0.005 | 0.129 | 0.021 | 0.768 | 0.615 | 0.591 | 0.585 | 0.429 | [symbol] |
| 0.004 | 0.214 | 0.043 | 0.264 | 0.005 | 0.129 | 0.017 | 0.798 | 0.615 | 0.591 | 0.495 | 0.429 | 1 [symbol] |
| 0.016 | 0.270 | 0.136 | 0.314 | 0.010 | 0.167 | 0.012 | 0.828 | 0.421 | 0.636 | 0.413 | 0.393 | 1 [symbol] |
| 0.022 | 0.285 | 0.169 | 0.357 | 0.012 | 0.197 | 0.012 | 0.798 | 0.366 | 0.636 | 0.375 | 0.357 | 1 [symbol] |
| 0.036 | 0.303 | 0.252 | 0.407 | 0.014 | 0.197 | 0.017 | 0.798 | 0.366 | 0.674 | 0.495 | 0.393 | 1 [symbol] |
| 0.095 | 0.337 | 0.423 | 0.426 | 0.033 | 0.197 | 0.024 | 0.768 | 0.315 | 0.674 | 0.633 | 0.420 | 1 [symbol] |
| 0.174 | 0.337 | 0.620 | 0.475 | 0.050 | 0.235 | 0.050 | 0.720 | 0.315 | 0.674 | 0.633 | 0.420 | 1 [symbol] |
| 0.202 | 0.356 | 0.703 | 0.475 | 0.050 | 0.235 | 0.050 | 0.720 | 0.269 | 0.674 | 0.633 | 0.420 | 1 [symbol] |
| 0.157 | 0.374 | 0.593 | 0.475 | 0.044 | 0.197 | 0.056 | 0.720 | 0.421 | 0.636 | 0.946 | 0.491 | 1 [symbol] |
| 0.019 | 0.322 | 0.136 | 0.357 | 0.014 | 0.167 | 0.021 | 0.768 | 0.315 | 0.629 | 0.633 | 0.420 | 1 [symbol] |
| 0.126 | 0.666 | 0.117 | 0.667 | 0.467 | 0.606 | 0.338 | 0.417 | 0.108 | 0.773 | 0.785 | 0.527 | 1 [symbol] |
| 0.066 | 0.666 | 0.032 | 0.692 | 0.614 | 0.568 | 0.459 | 0.386 | 0.108 | 0.720 | 0.219 | 0.348 | 1 [symbol] |
| 0.002 | 0.778 | 0.002 | 0.791 | 0.083 | 0.720 | 0.061 | 0.265 | 0.688 | 0.538 | 0.682 | 0.491 | 1 [symbol] |
| 0.008 | 0.759 | 0.004 | 0.791 | 0.314 | 0.682 | 0.384 | 0.417 | 0.191 | 0.667 | 0.152 | 0.321 | 1 [symbol] |
| 0.006 | 0.782 | 0.004 | 0.841 | 0.198 | 0.682 | 0.514 | 0.417 | 0.688 | 0.530 | 0.172 | 0.321 | 1 [symbol] |
| 0.089 | 0.651 | 0.039 | 0.717 | 0.737 | 0.568 | 0.794 | 0.477 | 0.132 | 0.720 | 0.029 | 0.250 | 1 [symbol] |
| 0.001 | 0.816 | 0.001 | 0.841 | 0.083 | 0.720 | 0.068 | 0.265 | 0.228 | 0.720 | 0.076 | 0.286 | 1 [symbol] |
| 0.000 | 0.834 | 0.001 | 0.841 | 0.050 | 0.720 | 0.015 | 0.235 | 0.421 | 0.583 | 0.733 | 0.455 | 1 [symbol] |
| 0.002 | 0.759 | 0.003 | 0.791 | 0.104 | 0.720 | 0.030 | 0.235 | 0.366 | 0.583 | 0.633 | 0.491 | 1 [symbol] |
| 0.023 | 0.741 | 0.022 | 0.767 | 0.263 | 0.644 | 0.139 | 0.356 | 0.366 | 0.629 | 0.133 | 0.321 | 1 [symbol] |
| 0.008 | 0.741 | 0.008 | 0.791 | 0.162 | 0.682 | 0.045 | 0.295 | 0.688 | 0.530 | 0.306 | 0.393 | 1 [symbol] |
| 0.012 | 0.741 | 0.008 | 0.767 | 0.287 | 0.644 | 0.258 | 0.356 | 0.366 | 0.621 | 0.246 | 0.357 | 1 [symbol] |
| 0.001 | 0.797 | 0.001 | 0.816 | 0.083 | 0.758 | 0.074 | 0.326 | 0.482 | 0.538 | 0.172 | 0.357 | 1 [symbol] |
| 0.044 | 0.684 | 0.147 | 0.643 | 0.065 | 0.720 | 0.037 | 0.265 | 0.269 | 0.364 | 0.076 | 0.732 | 1 [symbol] |
| 0.017 | 0.277 | 0.013 | 0.246 | 0.287 | 0.379 | 0.663 | 0.568 | 0.132 | 0.273 | 0.275 | 0.670 | 1 [symbol] |
| 0.003 | 0.247 | 0.003 | 0.209 | 0.117 | 0.273 | 0.151 | 0.659 | 0.228 | 0.318 | 0.891 | 0.527 | 1 [symbol] |
| 0.027 | 0.318 | 0.039 | 0.326 | 0.179 | 0.273 | 0.107 | 0.689 | 0.482 | 0.409 | 0.785 | 0.455 | 1 [symbol] |
| 0.031 | 0.314 | 0.032 | 0.252 | 0.263 | 0.341 | 0.207 | 0.629 | 0.315 | 0.318 | 0.785 | 0.554 | 1 [symbol] |
| 0.023 | 0.285 | 0.051 | 0.283 | 0.104 | 0.273 | 0.572 | 0.598 | 0.688 | 0.629 | 0.306 | 0.598 | 1 [symbol] |
| 0.016 | 0.281 | 0.036 | 0.258 | 0.093 | 0.273 | 0.317 | 0.629 | 0.841 | 0.545 | 0.682 | 0.527 | 1 [symbol] |
| 0.015 | 0.285 | 0.007 | 0.209 | 0.401 | 0.348 | 0.258 | 0.629 | 0.088 | 0.273 | 1.000 | 0.500 | 1 [symbol] |
| 0.044 | 0.299 | 0.016 | 0.233 | 0.654 | 0.417 | 0.296 | 0.616 | 0.044 | 0.182 | 0.682 | 0.563 | 1 [symbol] |
| 0.054 | 0.333 | 0.047 | 0.295 | 0.370 | 0.417 | 0.361 | 0.598 | 0.546 | 0.402 | 0.495 | 0.598 | 1 [symbol] |
| 0.036 | 0.295 | 0.020 | 0.227 | 0.467 | 0.417 | 0.223 | 0.629 | 0.191 | 0.318 | 0.946 | 0.527 | 1 [symbol] |
| 0.001 | 0.816 | 0.001 | 0.816 | 0.038 | 0.758 | 0.050 | 0.265 | 0.421 | 0.629 | 0.539 | 0.598 | [symbol] |
| 0.000 | 0.816 | 0.002 | 0.841 | 0.019 | 0.795 | 0.117 | 0.295 | 0.688 | 0.500 | 0.785 | 0.536 | [symbol] |
| 0.002 | 0.778 | 0.047 | 0.717 | 0.002 | 0.909 | 0.098 | 0.326 | 0.108 | 0.273 | 0.585 | 0.607 | [symbol] |
| 0.036 | 0.703 | 0.268 | 0.618 | 0.012 | 0.833 | 0.090 | 0.326 | 0.088 | 0.273 | 0.891 | 0.500 | [symbol] |
| 0.058 | 0.670 | 0.321 | 0.618 | 0.022 | 0.795 | 0.338 | 0.417 | 0.228 | 0.318 | 0.891 | 0.455 | [symbol] |
| 0.041 | 0.684 | 0.101 | 0.667 | 0.104 | 0.720 | 0.164 | 0.295 | 1.000 | 0.500 | 0.275 | 0.661 | [symbol] |
| 0.044 | 0.666 | 0.109 | 0.692 | 0.104 | 0.720 | 0.068 | 0.295 | 0.688 | 0.455 | 0.339 | 0.661 | [symbol] |
| 0.039 | 0.303 | 0.051 | 0.283 | 0.218 | 0.341 | 0.056 | 0.720 | 0.482 | 0.409 | 0.633 | 0.429 | [symbol] |
| 0.004 | 0.759 | 0.009 | 0.742 | 0.050 | 0.758 | 0.019 | 0.205 | 0.920 | 0.485 | 0.375 | 0.357 | [symbol] |
| 0.006 | 0.741 | 0.009 | 0.767 | 0.093 | 0.720 | 0.240 | 0.356 | 0.315 | 0.674 | 0.172 | 0.625 | [symbol] |
| 0.000 | 0.853 | 0.000 | 0.866 | 0.009 | 0.871 | 0.240 | 0.326 | 0.421 | 0.629 | 0.275 | 0.348 | [symbol] |
| 0.000 | 0.890 | 0.000 | 0.866 | 0.007 | 0.909 | 0.296 | 0.374 | 0.228 | 0.682 | 0.246 | 0.348 | [symbol] |
| 0.005 | 0.741 | 0.012 | 0.742 | 0.057 | 0.720 | 0.361 | 0.386 | 0.688 | 0.492 | 0.785 | 0.420 | [symbol] |
| 0.041 | 0.684 | 0.268 | 0.568 | 0.016 | 0.795 | 0.602 | 0.465 | 0.108 | 0.227 | 0.453 | 0.429 | [symbol] |
| 0.977 | 0.483 | 0.169 | 0.376 | 0.050 | 0.758 | 0.408 | 0.417 | 0.021 | 0.182 | 0.733 | 0.527 | [symbol] |
| 0.282 | 0.572 | 0.939 | 0.494 | 0.044 | 0.720 | 0.037 | 0.295 | 0.005 | 0.091 | 0.020 | 0.777 | [symbol] |
| 0.062 | 0.684 | 0.340 | 0.618 | 0.022 | 0.795 | 0.128 | 0.326 | 0.044 | 0.182 | 0.024 | 0.768 | [symbol] |
| 0.015 | 0.707 | 0.032 | 0.717 | 0.093 | 0.727 | 0.207 | 0.326 | 0.315 | 0.311 | 0.152 | 0.670 | [symbol] |
| 0.044 | 0.684 | 0.039 | 0.717 | 0.341 | 0.644 | 0.277 | 0.356 | 0.763 | 0.583 | 0.785 | 0.536 | 8 [symbol] |
| 0.008 | 0.759 | 0.008 | 0.791 | 0.162 | 0.644 | 0.296 | 0.417 | 0.615 | 0.583 | 0.585 | 0.536 | 9 [symbol] |
| 0.002 | 0.229 | 0.002 | 0.159 | 0.117 | 0.273 | 0.572 | 0.538 | 0.191 | 0.273 | 0.946 | 0.491 | 10 [symbol] |
| 0.008 | 0.251 | 0.008 | 0.215 | 0.198 | 0.348 | 0.602 | 0.586 | 0.366 | 0.364 | 0.219 | 0.321 | 10 [symbol] |
| 0.070 | 0.647 | 0.086 | 0.667 | 0.287 | 0.644 | 0.019 | 0.235 | 1.000 | 0.538 | 0.219 | 0.625 | 10 [symbol] |
| 0.034 | 0.684 | 0.032 | 0.742 | 0.287 | 0.606 | 0.223 | 0.356 | 0.482 | 0.583 | 0.133 | 0.670 | 10 [symbol] |
| 0.282 | 0.618 | 0.593 | 0.574 | 0.179 | 0.689 | 0.240 | 0.629 | 0.615 | 0.409 | 0.152 | 0.321 | 12 [symbol] |
| 0.001 | 0.154 | 0.001 | 0.134 | 0.065 | 0.273 | 0.192 | 0.598 | 0.108 | 0.273 | 0.633 | 0.598 | 13 [symbol] |
| 0.001 | 0.195 | 0.007 | 0.215 | 0.019 | 0.235 | 0.728 | 0.556 | 0.615 | 0.409 | 0.785 | 0.536 | 16 [symbol] |
| 0.269 | 0.393 | 0.760 | 0.500 | 0.083 | 0.273 | 0.408 | 0.568 | 0.159 | 0.720 | 0.041 | 0.214 | 18 [symbol] |

[symbol]

indicates data missing or illegible when filed

TABLE M
Biomarkers identified in F6ISL

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | M/ [symbol] |
| P | | P | | P | | P | | P | | P | | (avg [symbol] |
| value | ROC | value | ROC | value | ROC | value | ROC | value | ROC | value | ROC | D [symbol] |
| 0.427 | 0.580 | 0.500 | 0.553 | 0.568 | 0.542 | 0.840 | 0.549 | 0.920 | 0.492 | 0.050 | 0.281 | [symbol] |
| 0.012 | 0.717 | 0.003 | 0.792 | 0.568 | 0.583 | 0.518 | 0.569 | 0.132 | 0.712 | 0.245 | 0.344 | [symbol] |
| 0.068 | 0.647 | 0.095 | 0.686 | 0.254 | 0.667 | 0.353 | 0.403 | 0.688 | 0.583 | 0.178 | 0.313 | [symbol] |
| 0.427 | 0.462 | 0.619 | 0.473 | 0.407 | 0.375 | 0.024 | 0.757 | 0.841 | 0.500 | 0.159 | 0.313 | [symbol] |
| 0.020 | 0.717 | 0.051 | 0.712 | 0.087 | 0.708 | 0.196 | 0.326 | 0.228 | 0.311 | 0.759 | 0.531 | [symbol] |
| 0.691 | 0.521 | 0.227 | 0.606 | 0.325 | 0.333 | 0.052 | 0.736 | 0.191 | 0.712 | 0.426 | 0.406 | [symbol] |
| 0.771 | 0.501 | 0.722 | 0.473 | 0.276 | 0.625 | 0.903 | 0.438 | 0.228 | 0.311 | 0.245 | 0.375 | [symbol] |
| 0.053 | 0.678 | 0.102 | 0.659 | 0.162 | 0.667 | 0.419 | 0.354 | 0.920 | 0.500 | 0.358 | 0.375 | [symbol] |
| 0.214 | 0.384 | 0.241 | 0.394 | 0.468 | 0.417 | 0.115 | 0.687 | 0.763 | 0.492 | 0.426 | 0.406 | [symbol] |
| 0.255 | 0.392 | 0.644 | 0.473 | 0.120 | 0.292 | 0.293 | 0.410 | 0.421 | 0.591 | 0.178 | 0.375 | [symbol] |
| 0.037 | 0.333 | 0.088 | 0.314 | 0.108 | 0.292 | 0.225 | 0.375 | 0.920 | 0.538 | 1.000 | 0.500 | [symbol] |
| 0.057 | 0.333 | 0.060 | 0.288 | 0.325 | 0.375 | 0.492 | 0.569 | 0.841 | 0.447 | 0.951 | 0.531 | [symbol] |
| 0.177 | 0.412 | 0.005 | 0.182 | 0.147 | 0.667 | 0.210 | 0.625 | 0.007 | 0.091 | 0.327 | 0.406 | [symbol] |
| 0.937 | 0.501 | 0.831 | 0.527 | 0.876 | 0.458 | 0.353 | 0.403 | 0.920 | 0.583 | 0.426 | 0.375 | [symbol] |
| 0.032 | 0.294 | 0.095 | 0.314 | 0.078 | 0.292 | 0.182 | 0.667 | 0.688 | 0.583 | 0.426 | 0.406 | [symbol] |
| 0.112 | 0.638 | 0.522 | 0.606 | 0.029 | 0.792 | 0.657 | 0.451 | 0.159 | 0.273 | 0.426 | 0.375 | [symbol] |
| 0.427 | 0.588 | 0.145 | 0.659 | 0.568 | 0.458 | 0.157 | 0.653 | 0.366 | 0.629 | 0.806 | 0.469 | [symbol] |
| 0.007 | 0.717 | 0.006 | 0.792 | 0.213 | 0.667 | 0.196 | 0.326 | 0.191 | 0.720 | 0.806 | 0.469 | [symbol] |
| 0.024 | 0.697 | 0.394 | 0.580 | 0.002 | 0.917 | 0.057 | 0.299 | 0.012 | 0.136 | 0.126 | 0.688 | [symbol] |
| 0.383 | 0.580 | 0.522 | 0.606 | 0.437 | 0.583 | 0.097 | 0.354 | 0.763 | 0.455 | 0.298 | 0.625 | [symbol] |
| 0.002 | 0.776 | 0.001 | 0.845 | 0.195 | 0.667 | 0.840 | 0.465 | 0.269 | 0.667 | 1.000 | 0.500 | [symbol] |
| 0.000 | 0.815 | 0.002 | 0.818 | 0.010 | 0.875 | 0.903 | 0.493 | 0.269 | 0.356 | 0.327 | 0.625 | [symbol] |
| 0.014 | 0.737 | 0.030 | 0.739 | 0.097 | 0.708 | 0.135 | 0.326 | 0.920 | 0.500 | 0.426 | 0.406 | [symbol] |
| 0.021 | 0.697 | 0.009 | 0.765 | 0.468 | 0.625 | 0.135 | 0.354 | 0.070 | 0.765 | 0.854 | 0.438 | [symbol] |
| 0.057 | 0.678 | 0.028 | 0.765 | 0.604 | 0.583 | 0.353 | 0.438 | 0.108 | 0.720 | 0.426 | 0.625 | [symbol] |
| 0.015 | 0.717 | 0.004 | 0.792 | 0.604 | 0.583 | 0.097 | 0.326 | 0.315 | 0.667 | 0.501 | 0.563 | [symbol] |
| 0.008 | 0.737 | 0.001 | 0.818 | 0.641 | 0.542 | 0.069 | 0.243 | 0.108 | 0.758 | 0.759 | 0.531 | [symbol] |
| 0.011 | 0.737 | 0.001 | 0.845 | 0.756 | 0.542 | 0.135 | 0.326 | 0.108 | 0.758 | 0.903 | 0.469 | [symbol] |
| 0.001 | 0.795 | 0.000 | 0.845 | 0.133 | 0.750 | 0.005 | 0.188 | 0.012 | 0.856 | 0.501 | 0.406 | 4 [symbol] |
| 0.010 | 0.717 | 0.002 | 0.792 | 0.604 | 0.583 | 0.057 | 0.326 | 0.027 | 0.848 | 0.142 | 0.313 | 4 [symbol] |
| 0.034 | 0.678 | 0.065 | 0.712 | 0.147 | 0.708 | 0.241 | 0.382 | 1.000 | 0.492 | 0.245 | 0.375 | 4 [symbol] |
| 0.002 | 0.776 | 0.011 | 0.765 | 0.020 | 0.792 | 0.716 | 0.438 | 0.763 | 0.500 | 0.806 | 0.500 | 4 [symbol] |
| 0.024 | 0.678 | 0.017 | 0.712 | 0.351 | 0.625 | 0.069 | 0.299 | 0.159 | 0.712 | 0.540 | 0.594 | 4 [symbol] |
| 0.177 | 0.638 | 0.189 | 0.659 | 0.468 | 0.583 | 0.115 | 0.326 | 0.546 | 0.629 | 0.462 | 0.563 | 4 [symbol] |
| 0.244 | 0.619 | 0.500 | 0.580 | 0.195 | 0.667 | 0.467 | 0.403 | 0.482 | 0.402 | 0.668 | 0.594 | 4 [symbol] |
| 0.383 | 0.423 | 0.166 | 0.341 | 0.756 | 0.583 | 0.332 | 0.604 | 0.366 | 0.356 | 0.111 | 0.313 | 4 [symbol] |
| 0.014 | 0.294 | 0.006 | 0.235 | 0.407 | 0.375 | 0.275 | 0.618 | 0.191 | 0.318 | 0.270 | 0.344 | 6 [symbol] |
| 0.005 | 0.255 | 0.003 | 0.208 | 0.276 | 0.375 | 0.052 | 0.736 | 0.159 | 0.318 | 0.058 | 0.250 | 6 [symbol] |
| 0.003 | 0.255 | 0.002 | 0.182 | 0.254 | 0.375 | 0.353 | 0.625 | 0.108 | 0.273 | 0.358 | 0.375 | 8 [symbol] |
| 0.001 | 0.216 | 0.000 | 0.129 | 0.276 | 0.333 | 0.010 | 0.785 | 0.035 | 0.227 | 0.806 | 0.469 | 8 [symbol] |
| 0.015 | 0.275 | 0.006 | 0.208 | 0.468 | 0.417 | 0.075 | 0.701 | 0.088 | 0.227 | 0.462 | 0.406 | 8 [symbol] |
| 0.010 | 0.266 | 0.012 | 0.235 | 0.178 | 0.292 | 0.115 | 0.681 | 0.482 | 0.409 | 0.951 | 0.438 | 9 [symbol] |
| 0.042 | 0.305 | 0.030 | 0.261 | 0.407 | 0.375 | 0.135 | 0.653 | 0.191 | 0.318 | 0.903 | 0.500 | 9 [symbol] |
| 0.034 | 0.344 | 0.043 | 0.288 | 0.233 | 0.333 | 0.182 | 0.625 | 0.421 | 0.364 | 0.806 | 0.500 | 13 [symbol] |
| 0.020 | 0.305 | 0.016 | 0.235 | 0.300 | 0.375 | 0.063 | 0.681 | 0.366 | 0.364 | 0.540 | 0.406 | 17 [symbol] |
| 0.028 | 0.286 | 0.055 | 0.288 | 0.133 | 0.292 | 0.157 | 0.632 | 0.763 | 0.538 | 0.159 | 0.344 | 28 [symbol] |
| 0.026 | 0.294 | 0.051 | 0.288 | 0.133 | 0.333 | 0.146 | 0.681 | 0.920 | 0.492 | 0.086 | 0.281 | 34 [symbol] |
| 0.112 | 0.638 | 0.060 | 0.712 | 0.717 | 0.542 | 0.419 | 0.410 | 0.688 | 0.576 | 0.327 | 0.375 | 51 [symbol] |
| 0.026 | 0.294 | 0.023 | 0.261 | 0.300 | 0.375 | 0.210 | 0.653 | 0.688 | 0.409 | 0.358 | 0.375 | 66 [symbol] |
| 0.016 | 0.294 | 0.017 | 0.261 | 0.213 | 0.333 | 0.069 | 0.708 | 0.615 | 0.409 | 0.159 | 0.313 | 68 [symbol] |

[symbol]

indicates data missing or illegible when filed

TABLE N
Biomarkers identified in F6ISH

| | | | | | | Babesia | | | | | | |
|---|---|---|---|---|---|---|---|---|---|---|---|---|
| | | | | Babesia | | vs. | | Healthy | | | | |
| Babesia | | Babesia | | vs. | | Babesia | | vs. | | Babesia 1 | | |
| vs. Non- | | vs. | | Flu-like | | with/Lyme | | Flu-like | | vs. | | |
| babesia | | Healthy | | symptoms | | Disease | | symptoms | | Babesia 2 | | |
| P | | P | | P | | P | | P | | P | | M/Z [symbol] |
| value | ROC | value | ROC | value | ROC | value | ROC | value | ROC | value | ROC | D [symbol] |
| 0.019 | 0.267 | 0.007 | 0.164 | 0.551 | 0.411 | 0.150 | 0.651 | 0.126 | 0.291 | 0.063 | 0.247 | [symbol] |
| 0.021 | 0.267 | 0.008 | 0.189 | 0.551 | 0.411 | 0.217 | 0.682 | 0.234 | 0.291 | 0.033 | 0.208 | [symbol] |
| 0.032 | 0.288 | 0.022 | 0.237 | 0.412 | 0.411 | 0.304 | 0.651 | 0.193 | 0.291 | 0.042 | 0.208 | [symbol] |
| 0.025 | 0.288 | 0.012 | 0.215 | 0.502 | 0.411 | 0.111 | 0.735 | 0.193 | 0.291 | 0.052 | 0.208 | [symbol] |
| 0.030 | 0.309 | 0.013 | 0.237 | 0.551 | 0.411 | 0.181 | 0.651 | 0.157 | 0.291 | 0.077 | 0.247 | [symbol] |
| 0.035 | 0.306 | 0.025 | 0.237 | 0.412 | 0.411 | 0.198 | 0.651 | 0.193 | 0.291 | 0.077 | 0.247 | [symbol] |
| 0.025 | 0.288 | 0.015 | 0.237 | 0.412 | 0.411 | 0.150 | 0.682 | 0.234 | 0.291 | 0.042 | 0.208 | [symbol] |
| 0.032 | 0.288 | 0.022 | 0.237 | 0.412 | 0.411 | 0.150 | 0.704 | 0.282 | 0.345 | 0.042 | 0.208 | [symbol] |
| 0.045 | 0.309 | 0.031 | 0.263 | 0.456 | 0.411 | 0.111 | 0.651 | 0.234 | 0.345 | 0.042 | 0.208 | [symbol] |
| 0.042 | 0.309 | 0.017 | 0.240 | 0.655 | 0.456 | 0.136 | 0.713 | 0.079 | 0.236 | 0.189 | 0.325 | [symbol] |
| 0.062 | 0.330 | 0.022 | 0.240 | 0.823 | 0.456 | 0.304 | 0.620 | 0.100 | 0.291 | 0.390 | 0.403 | [symbol] |
| 0.062 | 0.330 | 0.017 | 0.265 | 0.941 | 0.456 | 0.411 | 0.590 | 0.079 | 0.236 | 0.497 | 0.364 | [symbol] |
| 0.078 | 0.351 | 0.025 | 0.265 | 0.941 | 0.456 | 0.304 | 0.620 | 0.079 | 0.236 | 0.390 | 0.364 | [symbol] |
| 0.053 | 0.330 | 0.015 | 0.240 | 0.881 | 0.494 | 0.217 | 0.651 | 0.079 | 0.236 | 0.221 | 0.364 | [symbol] |
| 0.010 | 0.267 | 0.003 | 0.189 | 0.456 | 0.367 | 0.217 | 0.651 | 0.100 | 0.291 | 0.441 | 0.403 | [symbol] |
| 0.006 | 0.267 | 0.002 | 0.189 | 0.371 | 0.367 | 0.045 | 0.744 | 0.062 | 0.236 | 0.160 | 0.325 | [symbol] |
| 0.027 | 0.306 | 0.019 | 0.263 | 0.371 | 0.322 | 0.072 | 0.691 | 0.157 | 0.291 | 0.113 | 0.286 | 1 [symbol] |
| 0.098 | 0.347 | 0.048 | 0.288 | 0.766 | 0.450 | 0.165 | 0.651 | 0.126 | 0.236 | 0.160 | 0.247 | 1 [symbol] |
| 0.084 | 0.326 | 0.039 | 0.263 | 0.766 | 0.450 | 0.165 | 0.682 | 0.079 | 0.236 | 0.556 | 0.442 | 1 [symbol] |
| 0.078 | 0.344 | 0.031 | 0.285 | 0.823 | 0.450 | 0.123 | 0.682 | 0.079 | 0.236 | 0.497 | 0.403 | 1 [symbol] |
| 0.053 | 0.330 | 0.031 | 0.265 | 0.551 | 0.411 | 0.051 | 0.704 | 0.234 | 0.291 | 0.063 | 0.247 | 1 [symbol] |
| 0.105 | 0.368 | 0.072 | 0.313 | 0.602 | 0.411 | 0.123 | 0.682 | 0.396 | 0.400 | 0.052 | 0.247 | 1 [symbol] |
| 0.035 | 0.694 | 0.043 | 0.687 | 0.233 | 0.672 | 0.136 | 0.352 | 0.777 | 0.545 | 0.751 | 0.558 | 1 [symbol] |
| 0.027 | 0.715 | 0.028 | 0.763 | 0.264 | 0.672 | 0.280 | 0.383 | 0.955 | 0.491 | 0.684 | 0.519 | 1 [symbol] |
| 0.049 | 0.694 | 0.096 | 0.687 | 0.136 | 0.672 | 0.719 | 0.475 | 0.777 | 0.509 | 0.751 | 0.481 | 1 [symbol] |
| 0.027 | 0.715 | 0.031 | 0.737 | 0.233 | 0.717 | 0.237 | 0.352 | 0.396 | 0.673 | 0.390 | 0.636 | 1 [symbol] |
| 0.045 | 0.715 | 0.053 | 0.687 | 0.264 | 0.672 | 0.537 | 0.475 | 0.777 | 0.564 | 0.684 | 0.519 | 1 [symbol] |
| 0.030 | 0.736 | 0.048 | 0.737 | 0.157 | 0.717 | 0.123 | 0.321 | 0.777 | 0.455 | 0.160 | 0.714 | 1 [symbol] |
| 0.007 | 0.736 | 0.022 | 0.712 | 0.044 | 0.761 | 0.607 | 0.444 | 0.955 | 0.455 | 0.221 | 0.714 | 1 [symbol] |
| 0.049 | 0.694 | 0.096 | 0.687 | 0.136 | 0.717 | 0.440 | 0.414 | 0.692 | 0.400 | 0.221 | 0.675 | 1 [symbol] |
| 0.038 | 0.694 | 0.150 | 0.636 | 0.037 | 0.806 | 0.959 | 0.506 | 0.396 | 0.400 | 0.342 | 0.597 | 1 [symbol] |
| 0.112 | 0.674 | 0.208 | 0.662 | 0.180 | 0.717 | 0.877 | 0.506 | 0.533 | 0.455 | 0.751 | 0.558 | 1 [symbol] |
| 0.042 | 0.694 | 0.072 | 0.712 | 0.157 | 0.717 | 0.607 | 0.475 | 0.865 | 0.491 | 0.189 | 0.714 | 1 [symbol] |
| 0.129 | 0.656 | 0.445 | 0.588 | 0.044 | 0.761 | 0.918 | 0.537 | 0.157 | 0.291 | 0.821 | 0.481 | 1 [symbol] |
| 0.049 | 0.698 | 0.065 | 0.715 | 0.233 | 0.628 | 0.537 | 0.383 | 0.692 | 0.564 | 0.298 | 0.636 | 1 [symbol] |
| 0.190 | 0.635 | 0.138 | 0.664 | 0.709 | 0.589 | 0.382 | 0.620 | 0.336 | 0.655 | 0.342 | 0.636 | 1 [symbol] |
| 0.012 | 0.740 | 0.005 | 0.813 | 0.412 | 0.589 | 0.918 | 0.497 | 0.396 | 0.655 | 0.821 | 0.519 | 1 [symbol] |
| 0.004 | 0.802 | 0.009 | 0.788 | 0.062 | 0.767 | 0.719 | 0.435 | 0.533 | 0.436 | 0.751 | 0.442 | 1 [symbol] |
| 0.013 | 0.740 | 0.004 | 0.813 | 0.551 | 0.589 | 0.918 | 0.528 | 0.396 | 0.600 | 0.964 | 0.519 | 1 [symbol] |
| 0.058 | 0.677 | 0.017 | 0.763 | 0.881 | 0.500 | 0.758 | 0.466 | 0.157 | 0.709 | 0.258 | 0.636 | 1 [symbol] |
| 0.049 | 0.698 | 0.048 | 0.737 | 0.333 | 0.589 | 0.797 | 0.537 | 0.777 | 0.600 | 0.497 | 0.597 | 1 [symbol] |
| 0.038 | 0.719 | 0.019 | 0.788 | 0.551 | 0.583 | 0.643 | 0.568 | 0.157 | 0.709 | 0.298 | 0.364 | 1 [symbol] |
| 0.073 | 0.656 | 0.017 | 0.763 | 0.941 | 0.494 | 0.237 | 0.682 | 0.100 | 0.764 | 0.821 | 0.519 | 1 [symbol] |
| 0.535 | 0.552 | 0.445 | 0.588 | 0.941 | 0.539 | 0.382 | 0.599 | 0.533 | 0.618 | 0.618 | 0.403 | 1 [symbol] |
| 0.679 | 0.531 | 0.393 | 0.588 | 0.602 | 0.450 | 0.198 | 0.630 | 0.336 | 0.618 | 0.342 | 0.636 | 1 [symbol] |
| 0.030 | 0.715 | 0.116 | 0.662 | 0.037 | 0.806 | 0.607 | 0.383 | 0.234 | 0.291 | 0.113 | 0.753 | 1 [symbol] |
| 0.730 | 0.549 | 0.787 | 0.535 | 0.766 | 0.539 | 0.382 | 0.599 | 0.865 | 0.455 | 0.094 | 0.714 | [symbol] |
| 0.704 | 0.451 | 0.472 | 0.588 | 0.044 | 0.233 | 0.100 | 0.321 | 0.020 | 0.873 | 0.821 | 0.519 | [symbol] |
| 0.227 | 0.368 | 0.787 | 0.462 | 0.031 | 0.189 | 0.440 | 0.444 | 0.011 | 0.927 | 0.751 | 0.558 | [symbol] |
| 0.255 | 0.410 | 0.787 | 0.462 | 0.044 | 0.189 | 0.959 | 0.528 | 0.015 | 0.927 | 0.342 | 0.325 | [symbol] |
| 0.017 | 0.271 | 0.096 | 0.316 | 0.017 | 0.194 | 0.797 | 0.506 | 0.157 | 0.727 | 0.189 | 0.364 | [symbol] |
| 0.007 | 0.250 | 0.031 | 0.268 | 0.025 | 0.194 | 0.217 | 0.651 | 0.777 | 0.564 | 0.077 | 0.247 | [symbol] |
| 0.005 | 0.226 | 0.006 | 0.215 | 0.118 | 0.278 | 0.111 | 0.682 | 0.396 | 0.400 | 0.033 | 0.208 | [symbol] |
| 0.011 | 0.247 | 0.017 | 0.240 | 0.118 | 0.278 | 0.123 | 0.642 | 0.610 | 0.455 | 0.094 | 0.286 | [symbol] |
| 0.004 | 0.799 | 0.008 | 0.813 | 0.074 | 0.761 | 0.681 | 0.444 | 0.777 | 0.545 | 0.684 | 0.519 | [symbol] |
| 0.000 | 0.861 | 0.001 | 0.864 | 0.021 | 0.850 | 0.471 | 0.414 | 0.396 | 0.673 | 0.892 | 0.519 | [symbol] |
| 0.084 | 0.635 | 0.243 | 0.639 | 0.074 | 0.717 | 0.382 | 0.599 | 0.234 | 0.345 | 0.160 | 0.714 | [symbol] |
| 0.010 | 0.757 | 0.025 | 0.763 | 0.062 | 0.806 | 0.837 | 0.444 | 0.777 | 0.455 | 0.002 | 0.909 | [symbol] |
| 0.023 | 0.736 | 0.015 | 0.737 | 0.371 | 0.628 | 0.382 | 0.404 | 0.157 | 0.709 | 0.892 | 0.519 | [symbol] |
| 0.019 | 0.740 | 0.003 | 0.838 | 0.881 | 0.500 | 0.304 | 0.620 | 0.011 | 0.873 | 0.892 | 0.481 | [symbol] |
| 0.005 | 0.760 | 0.015 | 0.740 | 0.037 | 0.806 | 0.837 | 0.537 | 0.692 | 0.564 | 0.892 | 0.481 | [symbol] |
| 0.067 | 0.677 | 0.138 | 0.662 | 0.136 | 0.722 | 0.150 | 0.651 | 0.955 | 0.491 | 0.342 | 0.597 | [symbol] |
| 0.138 | 0.639 | 0.345 | 0.591 | 0.101 | 0.717 | 0.040 | 0.765 | 0.234 | 0.291 | 0.160 | 0.714 | [symbol] |
| 0.010 | 0.778 | 0.053 | 0.712 | 0.017 | 0.850 | 0.643 | 0.568 | 0.462 | 0.345 | 0.160 | 0.714 | [symbol] |
| 0.013 | 0.247 | 0.010 | 0.240 | 0.264 | 0.361 | 0.035 | 0.744 | 0.282 | 0.345 | 0.964 | 0.481 | 1 [symbol] |
| 0.014 | 0.267 | 0.022 | 0.265 | 0.136 | 0.283 | 0.150 | 0.651 | 0.865 | 0.455 | 0.221 | 0.325 | 1 [symbol] |

[symbol]

indicates data missing or illegible when filed

TABLE O

| | | Fraction Protein Chip and |
|---|---|---|
| Marker M/Z (kDa) | P-Value | Beam Intensity |
| M7.06 | 0.028366 | F1CSL |
| M7.19 | 0.012611 | F1CSL |
| M7.16 | 0.001 | F1CSL |
| M7.48 | 0.005159 | F1CSL |
| M7.66 | 0.049366 | F1CSL |
| M8.93 | 0.049366 | F1CSL |
| M15.56 | 0.003012 | F1CSL |
| M44.23 | 0.000507 | F1CSL |
| M43.63 | 0.006502 | F1CSH |
| M146.62 | 0.005159 | F1CSH |
| M28.05 | 0.058782 | F3CSL |
| M44.43 | 0.049366 | F3CSH |
| M51.00 | 0.00236 | F4ISH |
| M66.27 | 0.02345 | F4ISH |
| M78.52 | 0.003219 | F4ISH |
| M99.25 | 0.01235 | F4ISH |
| M110.23 | 0.001856 | F4ISH |
| M131.82 | 0.017894 | F4ISH |
| M196.36 | 0.026897 | F4ISH |
| M14.37 | 0.028366 | F6CSL |
| M33.20 | 0.04125 | F6CSL |
| M45.08 | 0.005159 | F6CSL |
| M10.23 | 0.023342 | F1ISL |
| M10.27 | 0.015564 | F1ISL |
| M13.56 | 0.015564 | F1ISL |
| M43.21 | 0.034294 | F1ISL |
| M44.17 | 0.015564 | F1ISL |
| M44.42 | 0.01911 | F1ISL |
| M13.86 | 0.001499 | F4ISL |
| M22.22 | 0.006502 | F4ISL |
| M33.27 | 0.001499 | F4ISL |
| M44.25 | 0.003197 | F4ISL |

TABLE P
Identification of Certain Exemplary Biomarkers

| Molecular Weight | | |
|---|---|---|
| m/z Avg (kDa) | Fraction | Protein Identity |
| 11.1 | F5ISH | Ig Kappa Chain C region (allotype Inv(1, 2) - human |
| | | (fragment) - (SEQ ID NO: 12) |
| 12.7 | F1CSH | Ig heavy chain V region (anti-NDA, II-1) - human (fragment) |
| | | (SEQ ID NO: 14) |
| 15.9 | F1ISL | Hemoglobin, beta (SEQ ID NO: 21) |
| 16.5 | F1ISH | Chain B, Crystal structure of Human Hemogobin (SEQ ID |
| | | NO: 19) |
| 16.7 | F1ISH | Immunoglobulin alpha heavy chain variable region (SEQ ID |
| | | NO: 10) |
| 23.6 | F6CSH | Alpha-1-acid glycoprotein 1 precursor (HS) (SEQ ID NO: 22) |
| 26.5 | F1CSH | Lipoprotein Gln I (SEQ ID NO: 11) |
| 33.1 | F6CSH | Ig gamma-3 heavy chain disease proteins - human(SEQ ID |
| | | NO: 15) |
| 34 | F5ISL | Haptoglobin [contains haptoglobin alpha chain; haptoglobin |
| | | beta chain] (SEQ ID NO: 6) |
| 36.1 | F5ISL | Chain A, cleaved Alpha-1-Antitrypsin Polymer (SEQ ID |
| | | NO: 2) |
| 39.8 | F1CSL | Haptoglobin precursor [contains haptoglobin alpha chain; |
| | | haptoglobin beta chain] (SEQ ID NO: 7) |
| 39.9 | F1CSH | Preprohaptoglobin (SEQ ID NO: 5) |
| 41.3 | F1ISH | Haptoglobin Hp2 (SEQ ID NO: 13) |
| 43.5 | F5ISH | Chain A, Modified Alpha1-Antitrypsin (Modified Alpha1- |
| | | Proteinase Inhibitor) (Tetragonal Form 1) (SEQ ID NO: 3) |
| 51.5 | F1CSH | Kallistatin (SEQ ID NO: 18) |
| 51.9 | F5ISH | Alpha-1-antitrypsin precursor (SEQ ID NO: 1) |
| 52.5 | F5ISH | Alpha-1-antitrypsin precursor |
| 52.7 | F6ISH | Antithrombin III (SEQ ID NO: 8) |
| 53 | F6CSL | vitronectin precursor (serum spreading factor)(S |
| | | protein)(Glycoprotein 66) (SEQ ID NO: 9) |
| 79.2 | F1ISH | Transferrin (SEQ ID NO: 17) |
| 79.5 | F5CSL | Fibronectin |
| 99.6 | F5CSL | Ceruloplasmin (SEQ ID NO: 20) |
| 167.8 | F6CSH | Apolipoprotein B-100 precursor (SEQ ID NO: 16) |
| 168 | F6CSH | Complement C3 precursor (contains: complement C3 beta |
| | | chain, complement C3 alpha chain, C3a anaphyl) (SEQ ID |
| | | NO: 4) |

1-88. (canceled)

89. A method for qualifying babesia status in a subject comprising:
measuring at least one biomarker in a biological sample from the subject, wherein the at least one biomarker is selected from the group consisting of the biomarkers of Table 1, Table 2, and Table 3; and
correlating the measurement -with babesia status.

90. A method for determining the course of babesia comprising:
measuring, at a first time, at least one biomarker in a biological sample from the subject, wherein the at least one biomarker is selected from the group consisting of the biomarkers of Table 1, Table 2, and Table 3;
measuring, at a second time, the at least one biomarker in a biological sample from the subject; and
comparing the first measurement and the second measurement; wherein the comparative measurements determine the course of babesia.

91. A method comprising measuring at least one biomarker in a sample from a subject, wherein the at least one biomarker is selected from the group consisting of biomarkers of Table 1, Table 2, and Table 3.

92. A kit comprising:
a solid support comprising at least one capture reagent attached thereto, wherein the capture reagent binds at least one biomarker from a first group consisting of the biomarkers of Table 1, Table 2 and Table 3.

93. The kit of claim 92, wherein the solid support comprising a capture reagent is a SELDI probe.

94. The kit of claims 92 or 93, additionally comprising: a container containing at least one of the biomarkers of Table 1, Table 2, or Table 3.

95. The method of any one of claims 89 to 91 or the kits of claim 92 or 94, wherein the at least one biomarker is selected from the group consisting of biomarkers of molecular masses of about 2.8, 2.9, 3, 3.1, 3.2, 3.6, 3.8, 4, 4.1, 4.2, 4.3, 4.8, 4.9, 6.4, 7, 7.1, 7.2, 7.3, 7.5, 7.7, 7.9, 8.7, 8.8, 8.9, 10, 10.1, 10.2, 10.3, 10.4, 10.9, 11, 11.2, 11.3, 11.6, 11.8, 11.9, 12.6, 12.7, 12.8, 12.9, 13, 13.1, 13.2, 13.6, 13.8, 14.1, 14.4, 14.7, 15.1, 15.6, 15.9, 16.5, 16.7, 17.3, 17.8, 21.9, 22, 22.2, 22.3, 23.5, 23.6, 25.5, 25.8, 28, 28.1, 28.2, 33, 33.1, 33.2, 33.3, 34.1, 36.1, 39.8, 43.4, 44, 44.2, 44.3, 44.8, 45.1, 46.1, 47.7, 51, 53, 53.6, 60.6, 62.4, 66.9, 79, 18.1, 19.2, 22.3, 26.5, 39.6, 39.9, 40.1, 41.3, 43.2, 43.6, 44.2, 44.4, 44.6, 45.2, 44.7, 50, 50.5, 51.2, 51.5, 51.9, 52.5, 52.7, 58.9, 59.1, 59.6, 59.8, 60.5, 61.6, 61.9, 62.3, 62.8, 64, 66.3, 66.6, 78.5, 79, 79.2, 79.5, 79.6, 99.3, 99.6, 110.2, 131.8, 133.5, 134.6, 146, 146.6, 167.8, 168, and 196.4 kDa.

96. The method of any of claims 89, 90, or 95, further comprising: managing subject treatment based on the status.

97. The method of claim 96, further comprising: measuring the at least one biomarker after subject management and correlating the measurement with disease progression.

98. A composition comprising a purified biomolecule selected from the group consisting of the biomarkers of Table 1, Table 2, and Table 3.

99. A composition comprising a biospecific capture reagent that specifically binds a biomolecule selected from group consisting of the biomarkers of Table 1, Table 2, and Table 3.

100. A composition comprising a biospecific capture reagent bound to a biomarker of Table 1, Table 2, and Table 3.

101. A software product comprising: a) code that accesses data attributed to a sample, the data comprising measurement of at least one biomarker in the sample, the biomarker selected from the group consisting of the biomarkers of Table 1, Table 2, and Table 3; and b) code that executes a classification algorithm that classifies the <disease> status of the sample as a function of the measurement.

102. A method comprising detecting a biomarker of Table 1, Table 2, or Table 3 by mass spectrometry or immunoassay.

103. A method for identifying a compound that interacts with a biomarker of Table 1, Table 2 or Table 3 wherein said method comprises: a) contacting a biomarker of Table 1, Table 2, or Table 3 with a test compound; and b) determining whether the test compound interacts with a biomarker of Table 1, Table 2, or Table 3.


SEQUENCE LISTING (22 sequences)

SEQ ID NO: 1 - protein, 418 aa, Homo sapiens [sequence omitted]

SEQ ID NO: 2 - protein, 326 aa, Homo sapiens
KITPNLAEFAFSLYRQLAHQSSTNIFFSPVSIATAFAMLSLGTKADTHDEILEGLNFNLTEIPEAAIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENDRRSASLHLPKLSITGTYDLKSVLGQLGITKVFSNGADLSGVTEEAPLKLSKAVHKAVLTIDEKGTEAAGAMF

SEQ ID NO: 3 - protein, 347 aa, Homo sapiens
DTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPM

SEQ ID NO: 4 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNLRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 5 - protein, 347 aa, Homo sapiens
MSALGAVIALLLWGQLFAVDSGNDVTDIADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 6 - protein, 329 aa, Homo sapiens
EDTGSEATNNTEVSLPKPPVIENGYVEHMIRYQCKPFYKLHTEGDGVYTLNSEKHWTNKAVGEKLPECEAVCGKPKNPVDQVQRIMGGSVDAKGSFPWQAKMVSHHNLTSGATLINEQWLLTTAKNLFLGHKDDAKANDIAPTLKLYVGKNQLVEVEKVVLHPDYSKVDIGLIKLKQKVPIDERVMPICLPSKDYAEVGRIGYVSGWGRNSNFNFTELLKYVMLPVADQDKCVQHYEGSTVPEKKSPKSPVGVQPILNEHTFCAGMSKFQEDTCYGDAGSAFAVHDQDEDTWYAAGILSFDKSCTVAEYGVYVKVPSVLAWVQETIAGN

SEQ ID NO: 7 - protein, 347 aa, Homo sapiens
MRALGAVITLLLWGQLFAADFGNEVTDIADDSCPKPPEIANGYVEHLVRYQCKNYYRLRTEGDGVYALNSEKQWVNKAVGEQLPECEAVCGKPKHPVDQVQRIIGGSLDAKGSFPWQAKMVSRHNLVTGATLISEQWLLTTAKNLFLNHTENATAQDIAPTLTLYLGRRQLVEIEKVVLHPNYSEVDIGLIKLKDKVPVNERVMPICLPSKDYTEVGRVGYVSGWGRNSNFTYTDHLKYVMLPVADQDKCIQHYENSTVPENKIPKNPVGVQPILNEHTFCAGMSKYQEDTCYGDAGSTFAIHDLQQDTWYAAGILSFDKSCTVAEYGVYVKTFNILDWIQKTIASN

SEQ ID NO: 8 - protein, 464 aa, Homo sapiens [sequence omitted]

SEQ ID NO: 9 - protein, 475 aa, Homo sapiens
MAPLRPIFTLALLLWVVLADQESCKDRCTEGFNANRKCQCDELCSYYQSCCADYAAECKPQVTRGDVFTMPEDEYGPYDYIEQTKDNASVHAQPESPTVGQEPTLSPDLQTEGGAEPTHEVPLEPEMETLRPEGEDLQAGTTELGTSASPAEEELCSGKPFDAFTDLKNGSLFAFRGQYCYELDETAVRPGYPKLIQDVWGIEGPIDAAFTRINCQGKTYLFKGSQYWRFEDGILDPDYPRNISEGFSGIPDNVDAAFALPAHSYSGRERVYFFKGDKYWEYQFQQQPSQEECEGSSLSAVFEHFAMLHRDSWEDIFKLLFWGRPSGGARQPQFISRDWHGVPGKVDAAMAGRIYISGLTPSPSAKKQKSRRRSRKRYRSRYGRGRSQNSRRLSRSISRLWFSSEEVSLGPYNYEDYETSWLKPATSEPIQSVYFFSGDKYYRVNLRTQRVDTVNPPYPRSIAQYWLGCPAPGGQ

SEQ ID NO: 10 - protein, 160 aa, Homo sapiens
  MOD_RES (159)..(159): Any amino acid
QVQLVQSGAEVKKPGESLKISCRGSGYNFDTYWIEWVRQMPGKGLEWMGSIYPGDSETRYRPSLQGQVTMSADTSISTASLQWSSLKASDTAMYYCARLPPSAQSGNWYFDLWGRGTLVTVSSASPTSPKVFPLSLCSTPAQDGKRGSSPGPGSQGLSXP

SEQ ID NO: 11 - protein, 245 aa, Homo sapiens
DEPPQSPWDRVKDLATVYVDVLKDSGRDYVSQFQGSALGKQLNLKLLWDDVTSTFSKLRQELGPVTEEWFNDLQEKLNLEKETGELRQEMSKDLEEVKAKVQPYLDDFQKKWQEMELYRQKVEPLRAELQEGARQKLHELQEKLSPLGEEMRDRARAHVDALRTHLAPYSDELRQRLAARLEALKENGAGRLAEYHAKATEHLSTLSEKAKPALEDLRQGLLPVLESFKVSFLSALEEYTKLNTQ

SEQ ID NO: 12 - protein, 99 aa, Homo sapiens
FIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 13 - protein, 375 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGIL

SEQ ID NO: 14 - protein, 98 aa, Homo sapiens
EVQLVQSGAEVKKPGESLKISCKGSGYSFSTYWIGWVRQMPGKGLEWMGSVYPGDSDTTYSPSFQGQVTISADKCISTAYLQWSSLKASDTAMYYCAR

SEQ ID NO: 15 - protein, 289 aa, Homo sapiens
QMQGVNCTVSSELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVQVHNAKTKPREQQFNSTFRVVSVLTVLHQNWLDGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 16 - protein, 4563 aa, Homo sapiens [sequence omitted]

SEQ ID NO: 17 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 18 - protein, 427 aa, Homo sapiens
MHLIDYLLLLLVGLLALSHGQLHVEHDGESCSNSSHQQILETGEGSPSLKIAPANADFAFRFYYLIASETPGKNIFFSPLSISAAYAMLSLGACSHSRSQILEGLGFNLTELSESDVHRGFQHLLHTLNLPGHGLETRVGSALFLSHNLKFLAKFLNDTMAVYEAKLFHTNFYDTVGTIQLINDHVKKETRGKIVDLVSELKKDVLMVLVNYIYFKALWEKPFISSRTTPKDFYVDENTTVRVPMMLQDQEHHWYLHDRYLPCSVLRMDYKGDATVFFILPNQGKMREIEEVLTPEMLMRWNNLLRKRNFYKKLELHLPKFSISGSYVLDQILPRLGFTDLFSKWADLSGITKQQKLEASKSFHKATLDVDEAGTEAAAATTFAIKFFSAQTNRHILRFNRPFLVVIFSTSTQSVLFLGKVVDPTKP

SEQ ID NO: 19 - protein, 146 aa, Homo sapiens
VHLTPEEKSAVTALWGKVNVDEVGGKALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 20 - protein, 850 aa, Homo sapiens
VVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEGEYPDTK

SEQ ID NO: 21 - protein, 146 aa, Homo sapiens [sequence omitted]

SEQ ID NO: 22 - protein, 201 aa, Homo sapiens
MALSWVLTVPEPPTSAGSPDPIVCQPSTGAHHNATLDQITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES